(19) United States
(12) Patent Application Publication
(10) Pub. No.: US 2014/0030784 A1
(43) Pub. Date: Jan. 30, 2014

(54) Terpene Synthases and Methods of Using the Same

(71) Applicant: University of Tennessee Research Foundation, Knoxville, TN (US)

(72) Inventors: Feng Chen, Knoxville, TN (US); Guanglin Li, Xi'an (CN)

(21) Appl. No.: 13/954,824

(22) Filed: Jul. 30, 2013

Related U.S. Application Data
(60) Provisional application No. 61/677,308, filed on Jul. 30, 2012.

(51) Int. Cl. C12N 9/88; C12P 5/00

(52) U.S. Cl. CPC C12N 9/88; C12P 5/007
USPC 435/166; 536/23.2; 435/320.1; 435/252.3; 435/254.2; 435/419; 800/298; 800/278

(57) ABSTRACT

Disclosed are isolated nucleic acid molecules from Selaginella moellendorffii that encode a terpene synthase protein at least 80% identical to a protein encoded by the nucleic acid sequence according to any of SEQ ID NOs: 1-47 or a degenerate variant thereof or a functional fragment thereof. Isolated terpene synthase proteins from S. moellendorffii are also disclosed. Host cells transformed with the S. moellendorffii terpene synthase nucleic acids are also disclosed, for example cells of a single cell organism, such as bacteria and yeast, or multicellular organism, such as a plant. The host cells can be prokaryotic cells or eukaryotic cells. Transgenic plants, or any part thereof, stably transformed with S. moellendorffii terpene synthase nucleic acids are also disclosed In some examples the transgenic plant is a dicotyledon or a monocotyledon. A method is disclosed for producing a transgenic plant, as is a method for producing terpenes.

### CROSS-REFERENCE TO RELATED APPLICATION

This application claims the priority benefit of U.S. Provisional Application 61/677,308 filed on Jul. 30, 2012, which is incorporated by reference herein in its entirety.

### FIELD OF THE DISCLOSURE

This disclosure concerns the field of enzymology and in particular terpene synthases obtained from Selaginella moellendorffii and there use to produce terpenes.

### BACKGROUND

Terpenoids constitute the largest class of specialized (secondary) metabolites, with more than 55,000 individual compounds identified from all forms of life (Kasai et al., Nature 469:116-120, 2011). Many terpenoids are of plant origin, and they play diverse roles in the interactions of plants with their environment (Gershenzon and Dudareva, Nat Chem Biol 3:408-414, 2007). Terpene synthases (TPSs) are pivotal enzymes in terpenoid biosynthesis that catalyze the formation of the basic terpene skeletons from isoprenyl diphosphate precursors. In addition to plants, many species of bacteria and fungi also contain terpenoids and TPSs (Cane et al., Arch Biochem Biophys 300:416-422, 1993; Cane et al., Biochemistry 33:5846-5857, 1994; and Agger, et al., Mol Microbiol 72:1181-1195, 2009). Microbial TPSs, however, are only distantly related to plant TPSs (Cao et al., Proteins 78:2417-2432, 2010).

The presence of terpenoids in the plant kingdom has been investigated mainly in seed plants, which have been shown to produce a variety of size classes: hemiterpenes (C₅), monoterpenes (C₁₀), sesquiterpenes (C₁₅), and diterpenes (C₂₀). Similarly, the TPSs producing these classes can be categorized into hemiterpene synthases, monoterpene synthases, sesquiterpene synthases, and diterpene synthases, depending on the product formed. Knowledge about the evolution of TPSs, which are said to catalyze the most complex reactions in biology (Christianson, Curr Opin Chem Biol 12:141-150, 2008), is clearly important for understanding the evolution of terpenes and terpene diversity. Since the first functional elucidation of a plant TPS gene (Facchini and Chappell, Proc Natl Acad Sci USA 89:11088-11092, 1992), the number of TPS genes that has been isolated and functionally characterized from various plant species has grown exponentially. All characterized plant TPSs share significant sequence similarity with each other, implying a common evolutionary origin (Bohlmann et al., Proc Natl Acad Sci USA 95:4126-4133, 1998; Chen et al., Plant J 66:212-229, 2011). Of the several seed plants with genome sequences that have been determined, including Arabidopsis, poplar, grapevine, maize, rice, and sorghum, all possess a midsize TPS gene family of ˜30-100 functional members that include genes encoding all these classes of TPSs (with the exception of hemiterpene synthases) (Chen et al., 2011). In contrast, the genome of the moss Physcomitrella patens, the first nonseed plant to have its genome sequence determined (Rensing et al., Science 319:64-69, 2008), contains a single functional TPS gene encoding copalyl diphosphate synthase/kaurene synthase (CPS/KS). The P. patens CPS/KS (PpCPS/KS) is a bifunctional diterpene synthase catalyzing the consecutive reactions of geranylgeranyl diphosphate (GGPP) to copalyl diphosphate (CPP) and then CPP to ent-kaurene and ent-16α-hydroxykaurene (Hayashi et al., FEBS Lett 580:6175-6181, 2006).

Previous analysis of TPS gene structure and phylogeny led to the hypothesis that the ancestor of this gene class in plants is a diterpene synthase gene (Trapp and Croteau Genetics 158:811-832, 2001), likely resembling PpCPS/KS (Chen et al., 2011). Monoterpene and sesquiterpene synthases are shorter than diterpene synthases and have been hypothesized to have evolved from the ancestral diterpene synthase gene through the loss of an N-terminal domain (Trapp and Croteau, 2001; Keeling et al. Plant Physiol 152:1197-1208). The presence of a single diterpene synthase gene in P. patens on the one hand and several classes of TPSs in seed plants on the other hand raises the intriguing question of what evolutionary changes account for the vastly increased number and diversity of TPS genes in seed plants.

### SUMMARY OF THE DISCLOSURE

Disclosed are isolated nucleic acid molecules from Selaginella moellendorffii that encode a terpene synthase protein at least 80% identical to a protein encoded by the nucleic acid sequence according to any of SEQ ID NOs: 1-48 or a degenerate variant thereof or a functional fragment thereof. The disclosed terpene synthase nucleic acids can be operably linked to a promoter, for example as part of a nucleic acid construct and/or expression vector, which can confer an agronomic trait to a plant in which it is expressed, for example terpenoid production. Isolated terpene synthase proteins from S. moellendorffii are also disclosed.

Host cells transformed with the S. moellendorffii terpene synthase nucleic acids are also disclosed, for example cells of a single cell organism, such as bacteria and yeast, or a multicellular organism, such as a plant. The host cells can be prokaryotic cells or eukaryotic cells. Transgenic plants, or any part thereof, stably transformed with S. moellendorffii terpene synthase nucleic acids are also disclosed. In some examples, the transgenic plant is a dicotyledon or a monocotyledon. A method is disclosed for producing a transgenic plant as is a method for producing terpenes.

The foregoing and other features and advantages will become more apparent from the following detailed description of several embodiments, which proceeds with reference to the accompanying figures.

### BRIEF DESCRIPTION OF THE DRAWINGS

FIG. 1 is a phylogenetic tree constructed with the two types of Selaginella moellendorffii TPSs: those TPSs similar to other plant TPSs (SmTPSs) and those TPSs with microbial TPS-like sequences (SmMTPSLs). Also depicted are TPSs from other plants (other plant TPSs) and putative TPSs identified from bacteria (bacterial TPSs) and fungi (fungal TPSs).

FIG. 2A is a set of chromatograms showing S. moellendorffii TPSs are similar to other plant TPSs encode diterpene synthases. Chromatograms show the gas chromatography-mass spectrometry (GC-MS) analysis of terpenoids produced by recombinant SmTPS9 and SmTPS10 using GGPP as substrate. AtCPS, a known copalyl diphosphate synthase from Arabidopsis, was used as a positive control. Empty vector was used as a negative control. 1, GGPP hydrolysis product 1; 2, GGPP hydrolysis product 2; 3, copalol; 4, an additional hydrolysis product of copalyl diphosphate. Copalol is the dephosphorylated product of copalyl diphosphate. The mass spectrum of peak 4 is shown in FIG. 7.

FIG. 2B is a mass spectrum of peak 3 from SmTPS9 and mass spectrum of copalol produced by AtCPS.

FIG. 3A is a set of chromatograms showing the microbial type of TPSs in S. moellendorffii encodes sesquiterpene and monoterpene synthases. Chromatograms show the GC-MS analysis of terpenes produced by recombinant SmMTPSL22, SmMTPSL1, SmMTPSL17, and SmMTPSL26 using either geranyl diphosphate (GPP) or farnesyl diphosphate (FPP) as substrate. 1, limonene*; 2, linalool*; 3, (E)-nerolidol*; 4, α-copaene*; 5, β-elemene*; 6, γ-cadinene*; 7, δ-cadinene*; 8, unidentified oxygenated sesquiterpene; 9, unidentified sesquiterpene A; 10, unidentified sesquiterpene B; 11, 2-epi-(E)-β-caryophyllene; 12, germacrene D*; 13, bicyclogermacrene; 14, α-cadinene*. *Compounds were identified using authentic standards. All other compounds were tentatively identified based on the mass spectrum and Kovat's retention index. FIG. 8 shows chiral analysis of linalool, nerolidol, germacrene D, and β-elemene produced by SmMTPSLs.

FIG. 3B shows the structures of representative terpene products of SmMTPSLs.

FIG. 4 is a set of chromatograms showing the emission of monoterpenes and sesquiterpenes from S. moellendorffii plants. Chromatograms show the GC-MS analysis of the volatiles collected from the headspace of untreated S. moellendorffii plants and plants treated with a fungal elicitor alamethicin. Indicated peaks were identified to be terpenes, including the monoterpene linalool (1) and the sesquiterpenes β-elemene (2), germacrene D (3), β-sesquiphellandrene (4), and nerolidol (5). IS, internal standard.

FIG. 5 shows the intron/exon organization of 14 putative full-length SmTPS genes. Gene structures were plotted using the GSDS server (available on the world wide web at gsds.cbi.pku.edu.cn). The SmTPS4 is truncated because of limited space.

FIG. 6 shows the intron/exon organization of SmMTPSL genes. Gene structures of 48 SmMTPSLs were plotted using the GSDS server (available on the world wide web at gsds.cbi.pku.edu.cn). The third intron in SmMTPSL16 is truncated because of limited space.

FIG. 7 is a mass spectrum of an additional unknown hydrolysis product (peak 4) of copalyl diphosphate other than copalol.

FIGS. 8A-8D show the chiral analysis of linalool (A), nerolidol (B), germacrene D (C), and β-elemene (D) produced by the TPSs SmMTPSL22, SmMTPSL22, SmMTPSL17, and SmMTPSL1, respectively, in in vitro enzyme assays. In B and C, the chirality of nerolidol and germacrene D identified from the headspace of S. moellendorffii plants was also determined.

FIGS. 9A and 9B show the sequence analysis of SmMTPSL1 (A) and SmMTPSL26 (B) and their neighboring genes in the genome of S. moellendorffii. A genomic DNA fragment of 3,459 bp covering SmMTPSL1, a part of its neighboring gene, and the intergenic region were amplified by PCR and confirmed by sequencing. Similarly, a genomic DNA fragment of 3,145 bp covering SmMTPSL26, a part of its neighboring gene, and the intergenic region were amplified by PCR and confirmed by sequencing.

FIG. 10 shows the expression changes of four selected SmMTPSL genes in S. moellendorffii at 6 h after the treatment by alamethicin. Real-time PCR was performed to determine the expression changes of SmMTPSL1, SmMTPS17, SmMTPSL22, and SmMTPSL26 in alamethicin-treated and control S. moellendorffii plants. Expression values for individual genes were normalized to the levels of Sm6PGD expression in respective samples. The level of expression of individual genes in control tissues was arbitrarily set at 1.0.

FIG. 11 is a set of mass spectra showing sesquiterpene synthase activities with FPP.

FIG. 12 is a set of mass spectra showing monterpene synthase activities with GPP.

FIG. 13 is a set of mass spectra showing diterpene synthase activities with GGPP.

### BRIEF DESCRIPTION OF THE SEQUENCE LISTING

The nucleic acid sequences shown herein are shown using standard letter abbreviations for nucleotide bases, as defined in 37 C.F.R. 1.822. Only one strand of each nucleic acid sequence is shown, but the complementary strand is understood as included by any reference to the displayed strand. The Sequence Listing is submitted as an ASCII text file in the form of the file named UTK_{—}0121_ST25.txt, which was created on Jul. 28, 2013, is 197 kilobytes, and is incorporated by reference herein.

SEQ ID NOS: 1-47 are exemplary nucleic acid sequences of Selaginella moellendorffii terpene synthases from the SmMTPSL genes.

SEQ ID NOS: 48-89 are exemplary amino acid sequences of Selaginella moellendorffii terpene synthases from the SmMTPSL genes.

SEQ ID NOS; 90-97 are the nucleic acid sequences of primers.

### DETAILED DESCRIPTION

### I. Introduction

Terpene synthases (TPSs) are pivotal enzymes for the biosynthesis of terpenoids, the largest class of secondary metabolites made by plants and other organisms. To understand the basis of the vast diversification of these enzymes in plants, the inventors investigated Selaginella moellendorffii, a nonseed vascular plant. As disclosed herein, the genome of this species was found to contain two distinct types of TPS genes. The first type of genes, which was designated as S. moellendorffii TPS genes (SmTPSs), includes 18 members (SmTPS1-18). SmTPSs share common ancestry with typical seed plant TPSs. Selected members of the SmTPSs were shown to encode diterpene synthases. The second type of genes, designated as S. moellendorffii microbial TPS-like genes (SmMTPSLs), includes 48 members (SmMTPSL1-48). Phylogenetic analysis showed that SmMTPSLs are more closely related to microbial TPSs than other plant TPSs.

As detailed in the Examples below, selected SmMTPSLs were determined to function as monoterpene and sesquiterpene synthases. Many of the products formed were typical monoterpenes and sesquiterpenes that have been previously shown to be synthesized by classical plant TPS enzymes. Some in vitro products of the characterized SmMTPSLs were detected in the headspace of S. moellendorffii plants treated with the fungal elicitor alamethicin, showing that they are also formed in the intact plant.

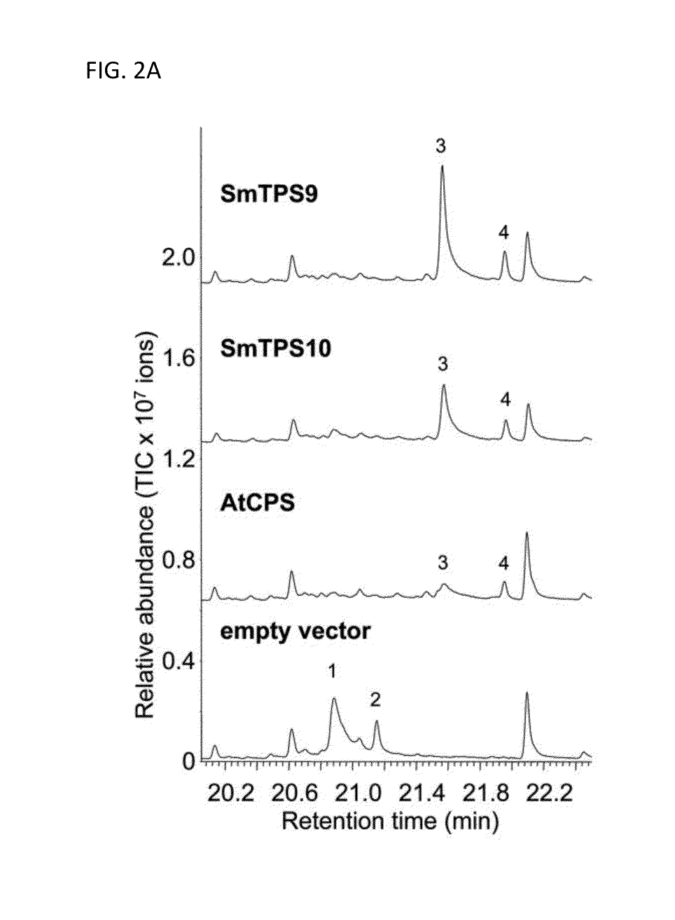

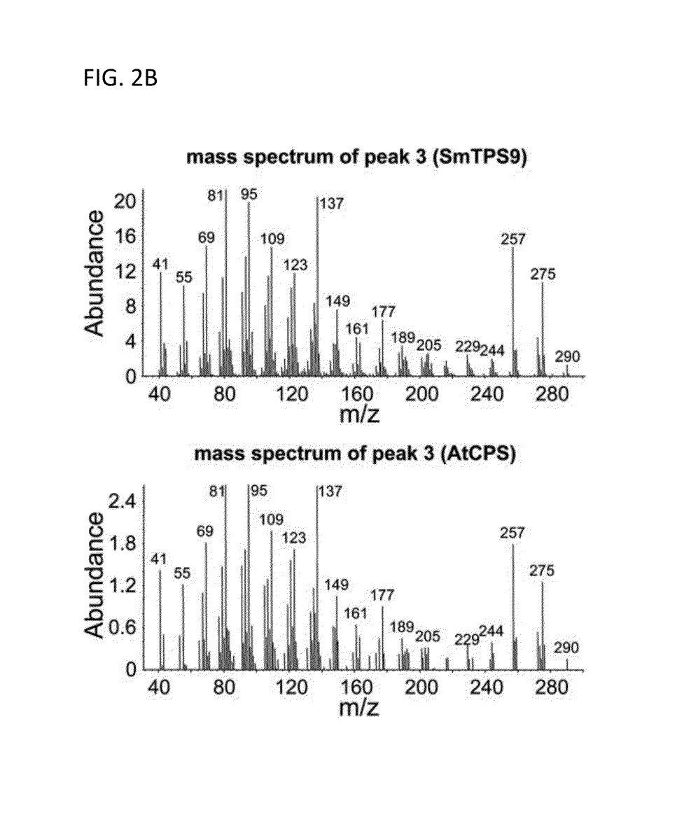

Interestingly, both types of TPSs in S. moellendorffii are functional. As shown in the Examples, SmTPS9 and SmTPS10 were determined to function as copalyl diphosphate synthases (FIG. 2). As monofunctional diterpene synthases, they convert GGPP to copalyl diphosphate, which is the substrate for gibberellins or other diterpenoids. In contrast, the previously characterized SmTPS7 and SmTPS4 function as bifunctional diterpene synthases, catalyzing the consecutive reactions of GGPP to copalyl diphosphate to final terpene products. These results indicate that S. moellendorffii contains both bifunctional and monofunctional diterpene synthases.

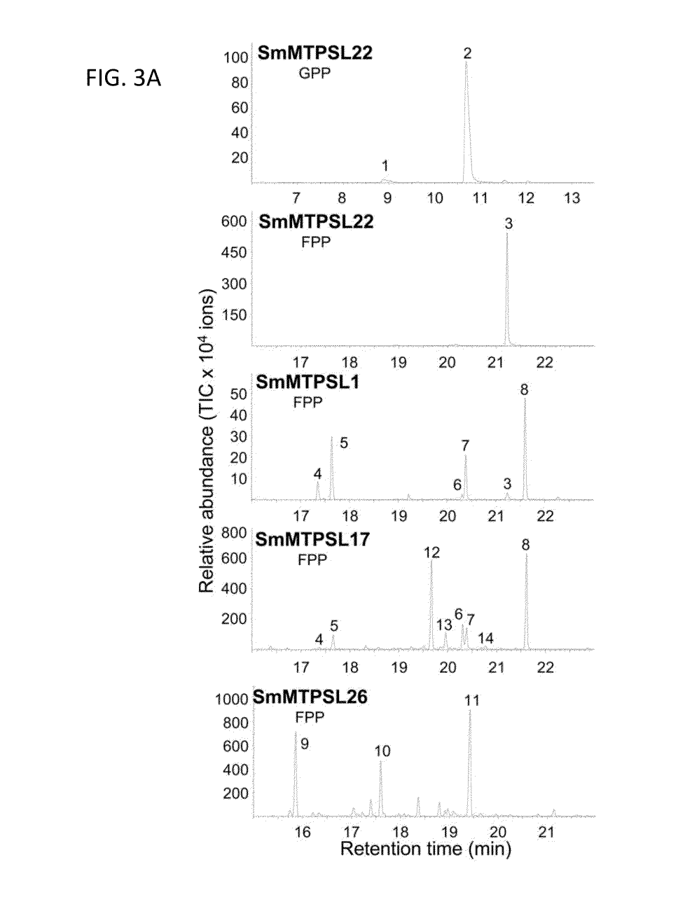

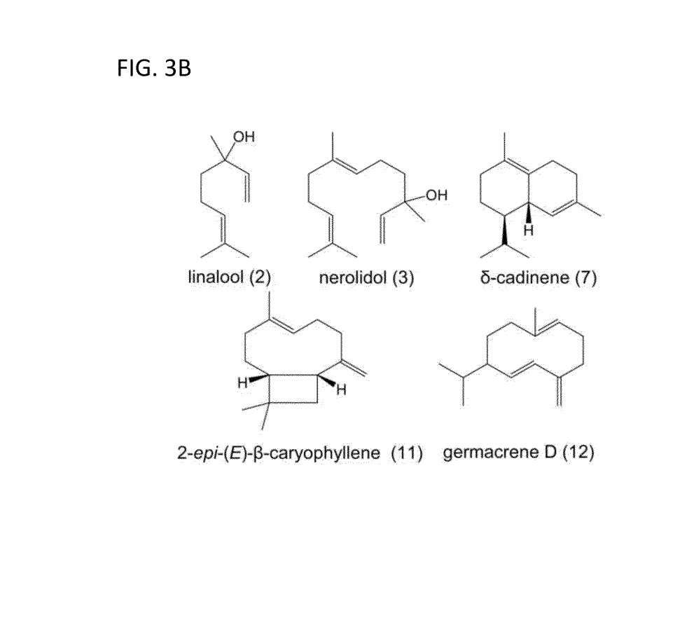

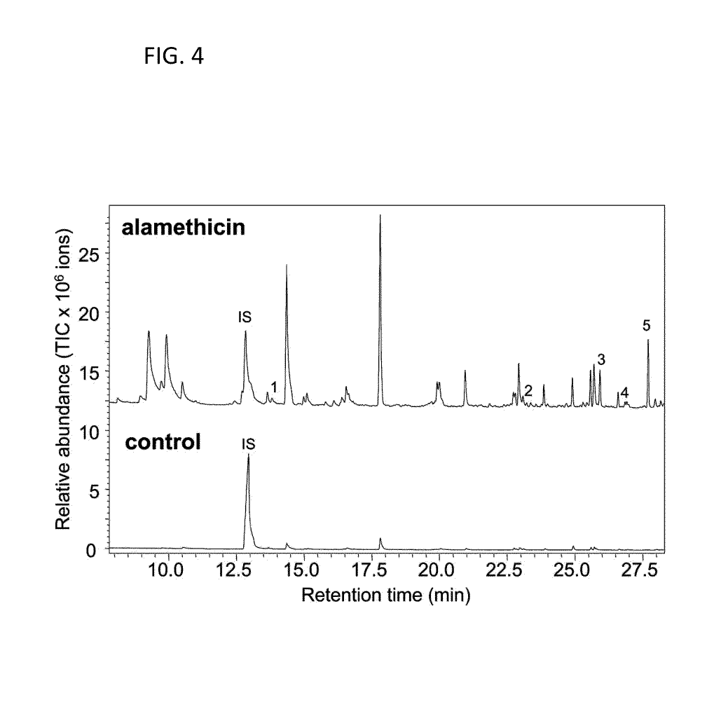

Selected SmMTPSLs, the microbial type TPSs, were also determined to be functional, displaying monoterpene synthase and sesquiterpene synthase activities (FIG. 3). Many products of the SmMTPSL enzymes that were tested, including linalool, (E)-nerolidol, α-copaene, β-elemene, γ-cadinene, δ-cadinene, 2-epi-(E)-β-caryophyllene, germacrene D, and α-cadinene, have been previously shown to be synthesized by many of the classical plant TPS enzymes. Some of these compounds, including linalool, germacrene D, and nerolidol, were also detected in the headspace of S. moellendorffii plants treated with the fungal elicitor alamethicin (FIG. 4), showing that these SmMTPSL products are also formed in the intact plant.

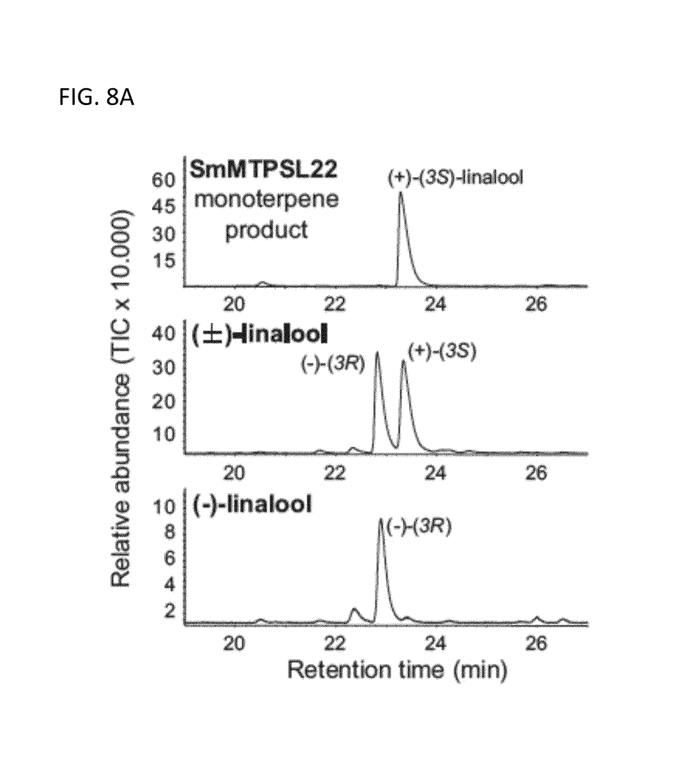

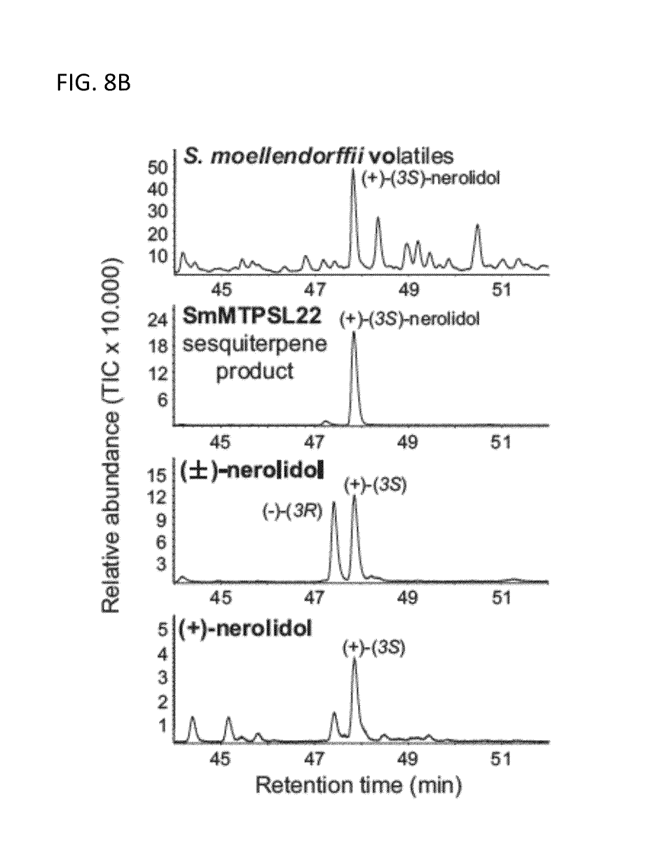

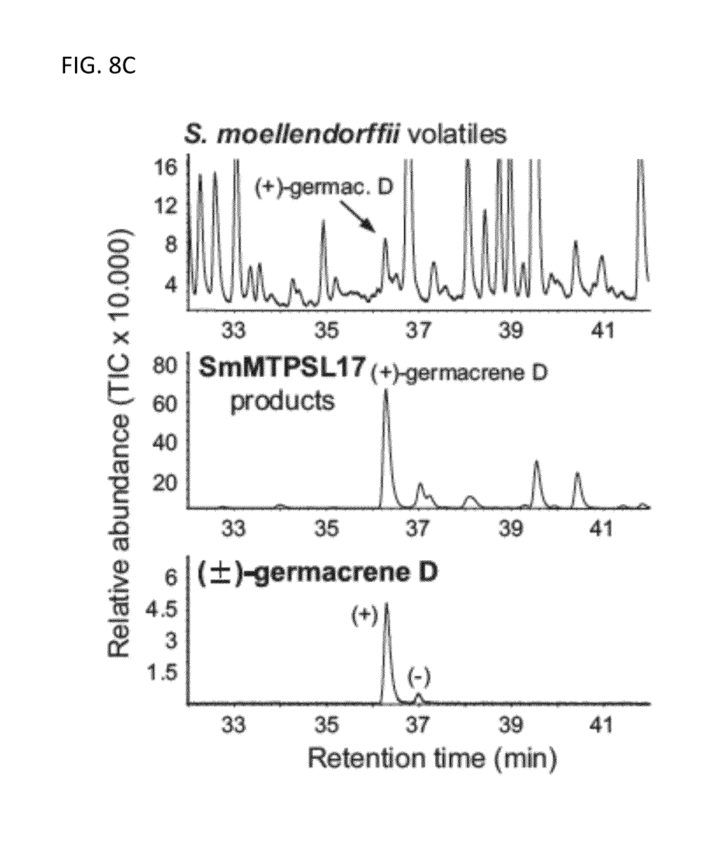

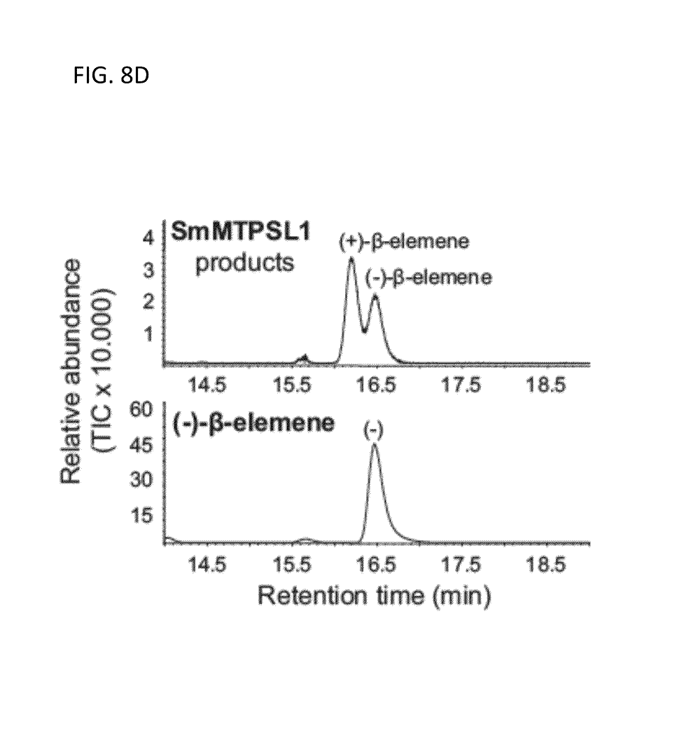

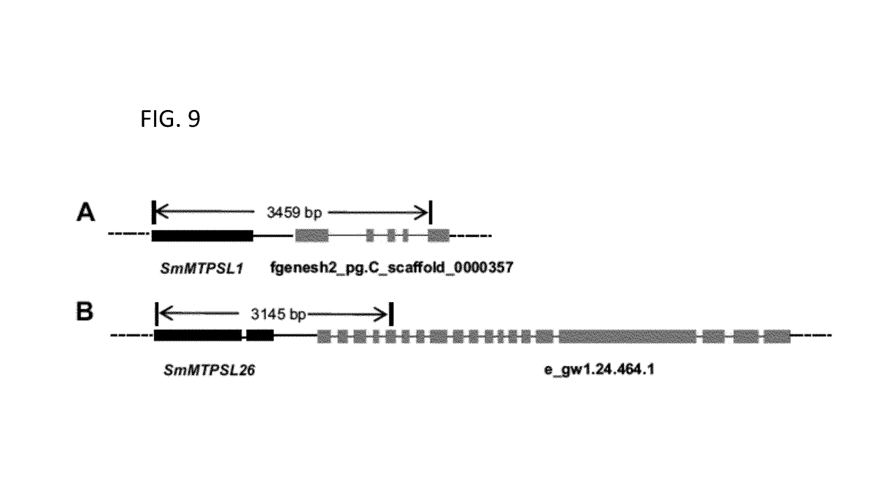

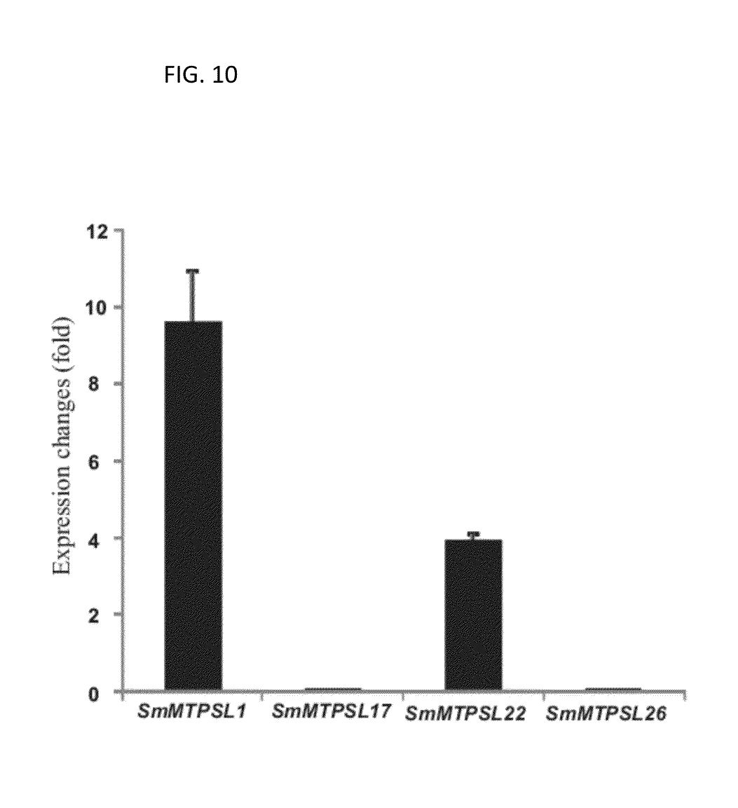

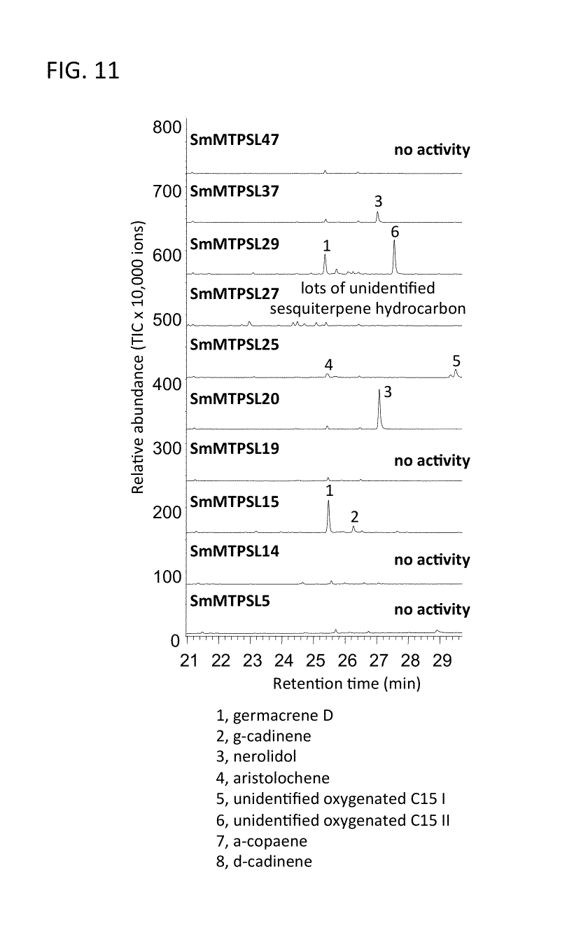

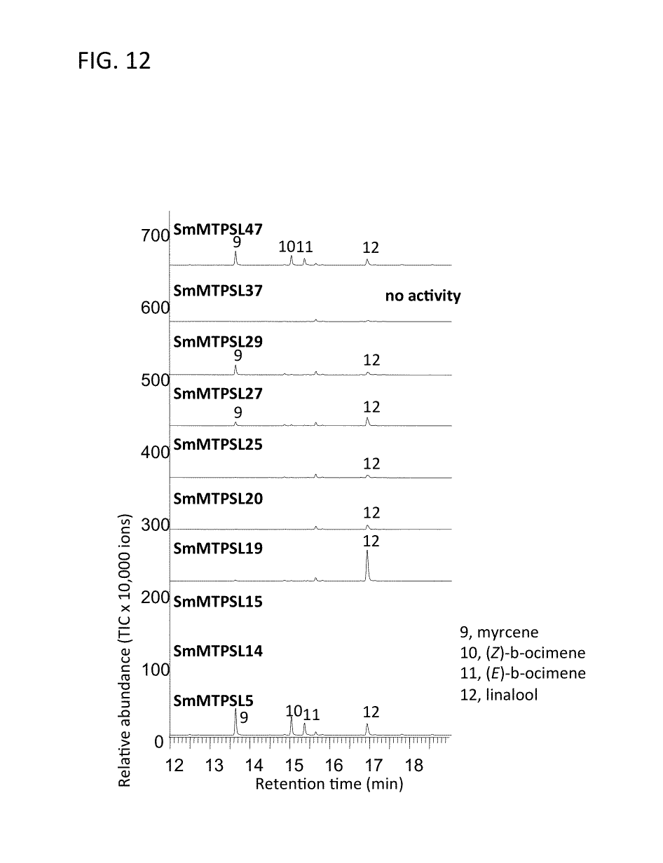

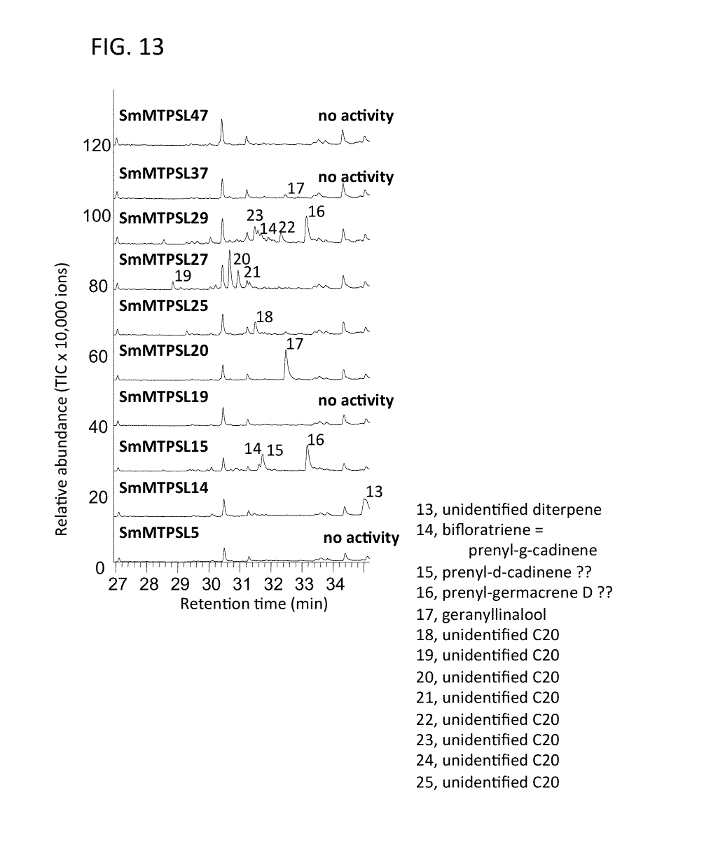

Moreover, in cases where the chirality of the headspace compounds was determinable, they always matched the chirality obtained in the in vitro enzyme assays (FIG. 8). In addition, the expression of some SmMTPSL genes was shown to be induced by the alamethicin treatment (FIG. 10) correlating with appearance of their products, providing additional evidence that the characterized SmMTPSL proteins function as genuine TPSs in S. moellendorffii. Because the alamethicin treatment mimics pathogen infection, the emission of terpenoids from S. moellendorffii after such treatment suggests that these chemicals, like in many seed plants, may have a role in plant defense.

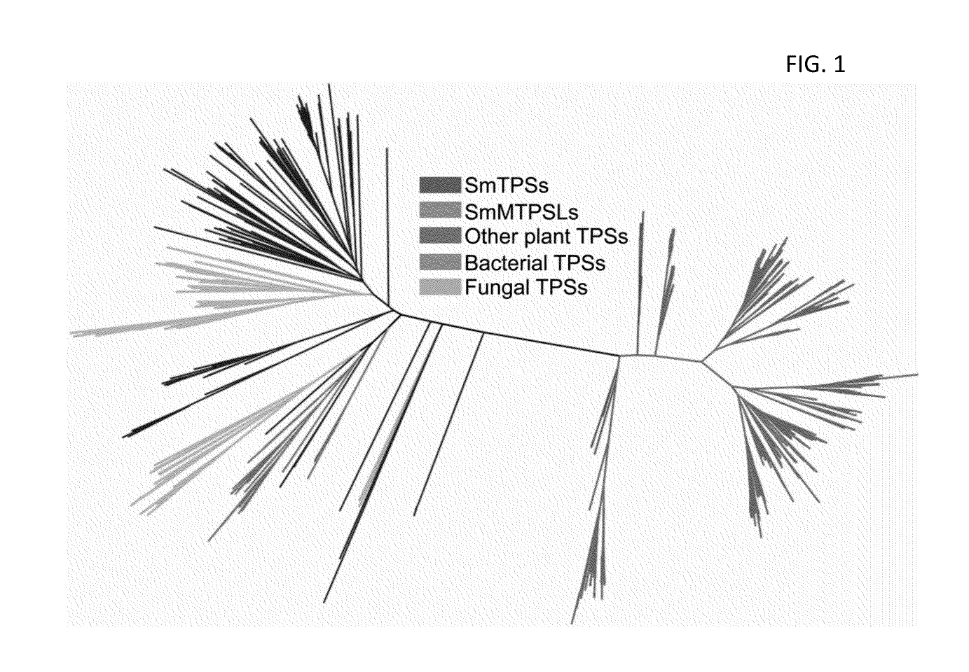

The presence of two types of TPSs with distinctive gene structures in S. moellendorffii poses intriguing questions about their evolutionary origins. The close similarity of SmTPSs to TPSs from other plants indicates that they are probably derived from a common TPS gene ancestor that was present in ancestral land plants (i.e., vertical transmission). However, SmMTPSLs are likely to have a different evolutionary origin based on their closer relationship to microbial TPSs than SmTPSs and other plant TPSs (FIG. 1). To the best of our knowledge, microbial TPS-like genes have not been found in other plant species, although so far, the only two genomes of nonseed plants available are those genomes of P. patens and S. moellendorffii. Two hypotheses can be invoked to explain the origin of SmMTPSLs. They may have been present in ancient land plants but were lost in P. patens and the seed plant lineages. Alternatively, an ancestral gene for SmMTPSLs may have been acquired by S. moellendorffii or its recent ancestor from microbes (and subsequently duplicated in the S. moellendorffii genome) through horizontal gene transfer, a mechanism where genetic material is moved across species other than by descent.

In summary, the data from bioinformatics approaches, phylogenetic methods, enzyme assays, and volatile metabolite analysis indicate that the S. moellendorffii genome contains two distinct groups of active TPSs, with SmTPSs functioning as diterpene synthases (FIG. 2) and SmMTPSLs functioning as monoterpene or sesquiterpene synthases (FIG. 3).

### II. Summary of Terms

Unless otherwise noted, technical terms are used according to conventional usage. Definitions of common terms in molecular biology may be found in Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); and Robert A. Meyers (ed.), Molecular Biology and Biotechnology: a Comprehensive Desk Reference, published by VCH Publishers, Inc., 1995 (ISBN 9780471185710).

The singular terms “a,” “an,” and “the” include plural referents unless context clearly indicates otherwise. Similarly, the word “or” is intended to include “and” unless the context clearly indicates otherwise. The term “comprises” means “includes.” In case of conflict, the present specification, including explanations of terms, will control.

To facilitate review of the various embodiments of this disclosure, the following explanations of terms are provided:

5′ and/or 3′: Nucleic acid molecules (such as, DNA and RNA) are said to have “5′ ends” and “3′ ends” because mononucleotides are reacted to make polynucleotides in a manner such that the 5′ phosphate of one mononucleotide pentose ring is attached to the 3′ oxygen of its neighbor in one direction via a phosphodiester linkage. Therefore, one end of a polynucleotide is referred to as the “5′ end” when its 5′ phosphate is not linked to the 3′ oxygen of a mononucleotide pentose ring. The other end of a polynucleotide is referred to as the “3′ end” when its 3′ oxygen is not linked to a 5′ phosphate of another mononucleotide pentose ring. Notwithstanding that a 5′ phosphate of one mononucleotide pentose ring is attached to the 3′ oxygen of its neighbor, an internal nucleic acid sequence also may be said to have 5′ and 3′ ends.

In either a linear or circular nucleic acid molecule, discrete internal elements are referred to as being “upstream” or 5′ of the “downstream” or 3′ elements. With regard to DNA, this terminology reflects that transcription proceeds in a 5′ to 3′ direction along a DNA strand. Promoter and enhancer elements, which direct transcription of a linked gene, are generally located 5′ or upstream of the coding region. However, enhancer elements can exert their effect even when located 3′ of the promoter element and the coding region. Transcription termination and polyadenylation signals are located 3′ or downstream of the coding region.

Agronomic trait: Characteristic of a plant, which characteristics include, but are not limited to, plant morphology, physiology, growth and development, yield, nutritional enhancement, disease or pest resistance, or environmental or chemical tolerance. In some examples an agronomic trait is the production of terpenes. An “enhanced agronomic trait” refers to a measurable improvement in an agronomic trait including, but not limited to, yield increase, including increased yield under non-stress conditions and increased yield under environmental stress conditions. Stress conditions may include, for example, drought, shade, fungal disease, viral disease, bacterial disease, insect infestation, nematode infestation, cold temperature exposure, heat exposure, osmotic stress, reduced nitrogen nutrient availability, reduced phosphorus nutrient availability and high plant density. “Yield” can be affected by many properties including without limitation, plant height, pod number, pod position on the plant, number of internodes, incidence of pod shatter, grain size, efficiency of nodulation and nitrogen fixation, efficiency of nutrient assimilation, resistance to biotic and abiotic stress, carbon assimilation, plant architecture, resistance to lodging, percent seed germination, seedling vigor, and juvenile traits.

Altering level of production or expression: Changing, either by increasing or decreasing, the level of production or expression of a nucleic acid molecule or an amino acid molecule (for example a gene, a polypeptide, a peptide), as compared to a control level of production or expression.

Amplification: When used in reference to a nucleic acid, this refers to techniques that increase the number of copies of a nucleic acid molecule in a sample or specimen. An example of amplification is the polymerase chain reaction, in which a biological sample collected from a subject is contacted with a pair of oligonucleotide primers, under conditions that allow for the hybridization of the primers to nucleic acid template in the sample. The primers are extended under suitable conditions, dissociated from the template, and then re-annealed, extended, and dissociated to amplify the number of copies of the nucleic acid. The product of in vitro amplification can be characterized by electrophoresis, restriction endonuclease cleavage patterns, oligonucleotide hybridization or ligation, and/or nucleic acid sequencing, using standard techniques. Other examples of in vitro amplification techniques include strand displacement amplification (see U.S. Pat. No. 5,744,311); transcription-free isothermal amplification (see U.S. Pat. No. 6,033,881); repair chain reaction amplification (see WO 90/01069); ligase chain reaction amplification (see EP-A-320 308); gap filling ligase chain reaction amplification (see U.S. Pat. No. 5,427,930); coupled ligase detection and PCR (see U.S. Pat. No. 6,027,889); and NASBA™ RNA transcription-free amplification (see U.S. Pat. No. 6,025,134).

Cassette: A manipulatable fragment of DNA carrying (and capable of expressing) one or more genes products of interest, for example expression of a terpene synthase disclosed herein, between one or more sets of restriction sites. A cassette can be transferred from one DNA sequence (usually on a vector) to another by “cutting” the fragment out using restriction enzymes and “pasting” it back into the new context.

cDNA (complementary DNA): A piece of DNA lacking internal, non-coding segments (introns) and transcriptional regulatory sequences. cDNA may also contain untranslated regions (UTRs) that are responsible for translational control in the corresponding RNA molecule. cDNA is usually synthesized in the laboratory by reverse transcription from messenger RNA extracted from cells or other samples. In some examples cDNA is used as a source of a nucleic acid of interest, such as a nucleic acid encoding a terpene synthase disclosed herein.

Construct: Any recombinant polynucleotide molecule such as a plasmid, cosmid, virus, autonomously replicating polynucleotide molecule, phage, or linear or circular single-stranded or double-stranded DNA or RNA polynucleotide molecule, derived from any source, capable of genomic integration or autonomous replication, comprising a polynucleotide molecule where one or more transcribable polynucleotide molecule, such as a nucleic acid, for example a cDNA encoding a disclosed terpene synthase, has been operably linked.

Control plant: A plant that does not contain a recombinant DNA that confers (for instance) an enhanced or altered agronomic trait in a transgenic plant, is used as a baseline for comparison, for instance in order to identify an enhanced or altered agronomic trait in the transgenic plant. A suitable control plant may be a non-transgenic plant of the parental line used to generate a transgenic plant, or a plant that at least is non-transgenic for the particular trait under examination (that is, the control plant may have been engineered to contain other heterologous sequences or recombinant DNA molecules). Thus, a control plant may in some cases be a transgenic plant line that comprises an empty vector or marker gene, but does not contain the recombinant DNA, or does not contain all of the recombinant DNAs, in the test plant.

Degenerate variant and conservative variant: A polynucleotide encoding a polypeptide or an antibody that includes a sequence that is degenerate as a result of the genetic code. There are 20 natural amino acids, most of which are specified by more than one codon. Therefore, all degenerate nucleotide sequences are included as long as the amino acid sequence of the polypeptide encoded by the nucleotide sequence is unchanged. Because of the degeneracy of the genetic code, a large number of functionally identical nucleic acids encode any given polypeptide. For instance, the codons CGU, CGC, CGA, CGG, AGA, and AGG all encode the amino acid arginine. Thus, at every position where an arginine is specified within a protein encoding sequence, the codon can be altered to any of the corresponding codons described without altering the encoded protein. Such nucleic acid variations are “silent variations,” which are one species of conservative variations. Each nucleic acid sequence herein that encodes a polypeptide also describes every possible silent variation. One of skill will recognize that each codon in a nucleic acid (except AUG, which is ordinarily the only codon for methionine) can be modified to yield a functionally identical molecule by standard techniques. Accordingly, each “silent variation” of a nucleic acid which encodes a polypeptide is implicit in each described sequence.

Furthermore, one of ordinary skill will recognize that individual substitutions, deletions or additions which alter, add or delete a single amino acid or a small percentage of amino acids (for instance less than 5%, such as less than 4%, less than 3%, less than 2%, or even less than 1%) in an encoded sequence are conservative variations where the alterations result in the substitution of an amino acid with a chemically similar amino acid.

Conservative amino acid substitutions providing functionally similar amino acids are well known in the art. The following six groups each contain amino acids that are conservative substitutions for one another:

1) Alanine (A), Serine (S), Threonine (T);

2) Aspartic acid (D), Glutamic acid (E);

3) Asparagine (N), Glutamine (Q);

4) Arginine (R), Lysine (K);

5) Isoleucine (I), Leucine (L), Methionine (M), Valine (V); and

6) Phenylalanine (F), Tyrosine (Y), Tryptophan (W).

Not all residue positions within a protein will tolerate an otherwise “conservative” substitution. For instance, if an amino acid residue is essential for a function of the protein, even an otherwise conservative substitution may disrupt that activity.

Disease resistance or pest resistance: The avoidance of the harmful symptoms that are the outcome of the plant-pathogen interactions. Disease resistance and pest resistance genes such as lysozymes or cecropins for antibacterial protection, or proteins such as defensins, glucanases or chitinases for antifungal protection, or Bacillus thuringiensis endotoxins, protease inhibitors, collagenases, lectins, or glycosidases for controlling nematodes or insects are all examples of useful gene products.

As used herein, the term “pest” includes, but is not limited to, insects, fungi, bacteria, viruses, nematodes, mites, ticks, and the like. Insect pests include insects selected from the orders Coleoptera, Diptera, Hymenoptera, Lepidoptera, Mallophaga, Homoptera, Hemiptera, Orthroptera, Thysanoptera, Dermaptera, Isoptera, Anoplura, Siphonaptera, Trichoptera, etc., particularly Coleoptera, Lepidoptera, and Diptera. Viruses include but are not limited to tobacco or cucumber mosaic virus, ringspot virus, necrosis virus, maize dwarf mosaic virus, etc. Nematodes include but are not limited to parasitic nematodes such as root knot, cyst, and lesion nematodes, including Heterodera spp., Meloidogyne spp., and Globodera spp.; particularly members of the cyst nematodes, including, but not limited to, Heterodera glycines (soybean cyst nematode); Heterodera schachtii (beet cyst nematode); Heterodera avenae (cereal cyst nematode); and Globodera rostochiensis and Globodera pallida (potato cyst nematodes). Lesion nematodes include but are not limited to Pratylenchus spp. Fungal pests include those that cause leaf, yellow, stripe and stem rusts.

DNA (deoxyribonucleic acid): DNA is a long chain polymer which comprises the genetic material of most organisms (some viruses have genes comprising ribonucleic acid (RNA)). The repeating units in DNA polymers are four different nucleotides, each of which comprises one of the four bases, adenine, guanine, cytosine and thymine bound to a deoxyribose sugar to which a phosphate group is attached. Triplets of nucleotides (referred to as codons) code for each amino acid in a polypeptide, or for a stop signal. The term codon is also used for the corresponding (and complementary) sequences of three nucleotides in the mRNA into which the DNA sequence is transcribed.

Unless otherwise specified, any reference to a DNA molecule includes the reverse complement of that DNA molecule. Except where single-strandedness is required by the text herein, DNA molecules, though written to depict only a single strand, encompass both strands of a double-stranded DNA molecule.

Encode: A polynucleotide is said to encode a polypeptide if, in its native state or when manipulated by methods known to those skilled in the art, the polynucleotide molecule can be transcribed and/or translated to produce a mRNA for and/or the polypeptide or a fragment thereof. The anti-sense strand is the complement of such a nucleic acid, and the encoding sequence can be deduced therefrom.

Enhancer domain: A cis-acting transcriptional regulatory element (a.k.a. cis-element) that confers an aspect of the overall control of gene expression. An enhancer domain may function to bind transcription factors, which are trans-acting protein factors that regulate transcription. Some enhancer domains bind more than one transcription factor, and transcription factors may interact with different affinities with more than one enhancer domain. Enhancer domains can be identified by a number of techniques, including deletion analysis (deleting one or more nucleotides from the 5′ end or internal to a promoter); DNA binding protein analysis using DNase I foot printing, methylation interference, electrophoresis mobility-shift assays, in vivo genomic foot printing by ligation-mediated PCR, and other conventional assays; or by DNA sequence comparison with known cis-element motifs using conventional DNA sequence comparison methods. The fine structure of an enhancer domain can be further studied by mutagenesis (or substitution) of one or more nucleotides or by other conventional methods. Enhancer domains can be obtained by chemical synthesis or by isolation from promoters that include such elements, and they can be synthesized with additional flanking nucleotides that contain useful restriction enzyme sites to facilitate subsequence manipulation.

Expression Control Sequences: Nucleic acid sequences that regulate the expression of a heterologous nucleic acid sequence to which it is operatively linked, for example the expression of a terpene synthase nucleic acid encoding a protein operably linked to expression control sequences. Expression control sequences are operatively linked to a nucleic acid sequence when the expression control sequences control and regulate the transcription and, as appropriate, translation of the nucleic acid sequence. Thus expression control sequences can include appropriate promoters, enhancers, transcription terminators, a start codon (ATG) in front of a protein-encoding gene, splicing signal for introns, maintenance of the correct reading frame of that gene to permit proper translation of mRNA, and stop codons. The term “control sequences” is intended to include, at a minimum, components whose presence can influence expression, and can also include additional components whose presence is advantageous, for example, leader sequences and fusion partner sequences. Expression control sequences can include a promoter.

A promoter is a minimal sequence sufficient to direct transcription. Also included are those promoter elements which are sufficient to render promoter-dependent gene expression controllable for cell-type specific, tissue-specific, or inducible by external signals or agents; such elements may be located in the 5′ or 3′ regions of the gene. Both constitutive and inducible promoters are included (see for example, Bitter et al., Methods in Enzymology 153:516-544, 1987). For example, when cloning in bacterial systems, inducible promoters such as pL of bacteriophage lambda, plac, ptrp, ptac (ptrp-lac hybrid promoter) and the like may be used. Promoters produced by recombinant DNA or synthetic techniques may also be used to provide for transcription of the nucleic acid sequences.

A polynucleotide can be inserted into an expression vector that contains a promoter sequence, which facilitates the efficient transcription of the inserted genetic sequence of the host. The expression vector typically contains an origin of replication, a promoter, as well as specific nucleic acid sequences that allow phenotypic selection of the transformed cells.

(Gene) Expression: Transcription of a DNA molecule into a transcribed RNA molecule. More generally, gene expression encompasses the processes by which a gene's coded information is converted into the structures present and operating in the cell. Expressed genes include those that are transcribed into mRNA and then translated into protein and those that are transcribed into RNA but not translated into protein (for example, siRNA, transfer RNA and ribosomal RNA). Thus, expression of a target sequence, such as a gene or a promoter region of a gene, can result in the expression of an mRNA, a protein, or both. The expression of the target sequence can be inhibited or enhanced (decreased or increased). Gene expression may be described as related to temporal, spatial, developmental, or morphological qualities as well as quantitative or qualitative indications.

Gene regulatory activity: The ability of a polynucleotide to affect transcription or translation of an operably linked transcribable polynucleotide molecule, such as an inducible promoter. An isolated polynucleotide molecule having gene regulatory activity may provide temporal or spatial expression or modulate levels and rates of expression of the operably linked transcribable polynucleotide molecule. An isolated polynucleotide molecule having gene regulatory activity may include a promoter, intron, leader, or 3′ transcription termination region.

Genetic material: A phrase meant to include all genes, nucleic acid, DNA and RNA.

Heterologous nucleotide sequence: A sequence that is not naturally occurring with a promoter sequence. While this nucleotide sequence is heterologous to the promoter sequence, it may be homologous, or native, or heterologous, or foreign, to the plant host. The invention additionally encompasses expression of the homologous coding sequences of the promoters, particularly the coding sequences related to the resistance phenotype. The expression of the homologous coding sequences will alter the phenotype of the transformed plant or plant cell.

Host cells: Cells in which a vector can be propagated and its nucleic acids expressed. The cell may be prokaryotic or eukaryotic. The term also includes any progeny of the subject host cell. It is understood that all progeny may not be identical to the parental cell since there may be mutations that occur during replication. However, such progeny are included when the term “host cell” is used.

Increasing pest resistance or enhancing pest resistance: An enhanced or elevated resistance to a past over a normal or control plant or part thereof (for example a plant that has not been transformed with an isolated nucleic acid encoding a terpene synthase disclosed herein). In some examples, an increase or enhancement is an elevation of at least about 25%, 50%, 75%, 100%, 150%, 200%, 300%, 400%, 500% or more.

In cis: Indicates that two sequences are positioned on the same piece of RNA or DNA.

In trans: Indicates that two sequences are positioned on different pieces of RNA or DNA.

Insert DNA: Heterologous DNA within an expression cassettes, such as the disclosed expression cassette, used to transform the plant material while “flanking DNA” can comprise either genomic DNA naturally present in an organism such as a plant, or foreign (heterologous) DNA introduced via the transformation process which is extraneous to the original insert DNA molecule, e.g. fragments associated with the transformation event. A “flanking region” or “flanking sequence” as used herein refers to a sequence of at least 20, 50, 100, 200, 300, 400, 1000, 1500, 2000, 2500, or 5000 base pair or greater which is located either immediately upstream of and contiguous with or immediately downstream of and contiguous with the original foreign insert DNA molecule.

Isolated: An “isolated” biological component (such as a nucleic acid, peptide or protein) has been substantially separated, produced apart from, or purified away from other biological components in the cell of the organism in which the component naturally occurs, e.g., other chromosomal and extrachromosomal DNA and RNA, and proteins. Nucleic acids, peptides and proteins which have been “isolated” thus include nucleic acids and proteins purified by standard purification methods. The term also embraces nucleic acids, peptides and proteins prepared by recombinant expression in a host cell as well as chemically synthesized nucleic acids.

Polypeptide: Any chain of amino acids, regardless of length or post-translational modification (such as glycosylation or phosphorylation). “Polypeptide” applies to amino acid polymers to naturally occurring amino acid polymers and non-naturally occurring amino acid polymer as well as in which one or more amino acid residue is a non-natural amino acid, for example an artificial chemical mimetic of a corresponding naturally occurring amino acid. In some embodiments, the polypeptide is a S. moellendorffii terpene synthase polypeptide. A “residue” refers to an amino acid or amino acid mimetic incorporated in a polypeptide by an amide bond or amide bond mimetic. A polypeptide has an amino terminal (N-terminal) end and a carboxy terminal (C-terminal) end. “Polypeptide” is used interchangeably with peptide or protein, and is used interchangeably herein to refer to a polymer of amino acid residues.

Nucleic acid (molecule or sequence): A deoxyribonucleotide or ribonucleotide polymer including without limitation, cDNA, mRNA, genomic DNA, and synthetic (such as chemically synthesized) DNA or RNA. The nucleic acid can be double stranded (ds) or single stranded (ss). Where single stranded, the nucleic acid can be the sense strand or the antisense strand. Nucleic acids can include natural nucleotides (such as A, T/U, C, and G), and can include analogs of natural nucleotides, such as labeled nucleotides. In some examples, a nucleic acid is a S. moellendorffii terpene synthase nucleic acid, which can include nucleic acids purified from S. moellendorffii as well as the amplification products of such nucleic acids.

Operably linked: This term refers to a juxtaposition of components, particularly nucleotide sequences, such that the normal function of the components can be performed. Thus, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein-coding regions, in the same reading frame. A coding sequence that is “operably linked” to regulatory sequence(s) refers to a configuration of nucleotide sequences wherein the coding sequence can be expressed under the regulatory control (e.g., transcriptional and/or translational control) of the regulatory sequences.

Plant: Any plant and progeny thereof. The term also includes parts of plants, including seed, cuttings, tubers, fruit, flowers, etc. As used herein, the term plant includes plant cells, plant organs, plant protoplasts, plant cell tissue cultures from which plants can be regenerated, plant calli, plant clumps, and plant cells that are intact in plants or parts of plants such as embryos, pollen, ovules, seeds, leaves, flowers, branches, fruit, stalks, roots, root tips, anthers, and the like. Progeny, variants, and mutants of the regenerated plants are also included within the scope of the invention. The term plant cell, as used herein, refers to the structural and physiological unit of plants, consisting of a protoplast and the surrounding cell wall, including those with genetic alteration, such as transformation, has been affected as to a gene of interest, or is a plant or plant cell which is descended from a plant or cell so altered and which comprises the alteration. A “control” or “control plant” or “control plant cell” provides a reference point for measuring changes in phenotype of the subject plant or plant cell. A control plant or plant cell may comprise, for example: (a) a wild-type plant or cell, i.e., of the same genotype as the starting material for the genetic alteration which resulted in the subject plant or cell; (b) a plant or plant cell of the same genotype as the starting material but which has been transformed with a null construct (i.e. with a construct which has no known effect on the trait of interest, such as a construct comprising a marker gene); (c) a plant or plant cell which is a non-transformed segregant among progeny of a subject plant or plant cell; (d) a plant or plant cell genetically identical to the subject plant or plant cell but which is not exposed to conditions or stimuli that would induce expression of the gene of interest; or (e) the subject plant or plant cell itself, under conditions in which the gene of interest is not expressed. The term plant organ, as used herein, refers to a distinct and visibly differentiated part of a plant, such as root, stem, leaf or embryo. More generally, the term plant tissue refers to any tissue of a plant in planta or in culture. This term includes a whole plant, plant cell, plant organ, protoplast, cell culture, or any group of plant cells organized into a structural and functional unit.

Promoter: An array of nucleic acid control sequences which direct transcription of a nucleic acid, by recognition and binding of e.g., RNA polymerase II and other proteins (trans-acting transcription factors) to initiate transcription. A promoter includes necessary nucleic acid sequences near the start site of transcription, such as, in the case of a polymerase II type promoter, a TATA element. Minimally, a promoter typically includes at least an RNA polymerase binding site together with one or more transcription factor binding sites, which modulate transcription in response to occupation by transcription factors. Representative examples of promoters (and elements that can be assembled to produce a promoter) are described herein. Promoters may be defined by their temporal, spatial, or developmental expression pattern.

A plant promoter is a native or non-native promoter that is functional in plant cells. In one example, a promoter is a high level constitutive promoter, such as a tissue specific promoter.

Protein: A biological molecule, for example a polypeptide, expressed by a gene and comprised of amino acids.

Protoplast: An isolated plant cell without cell walls, having the potential for regeneration into cell culture or a whole plant.

Purified: The term purified does not require absolute purity; rather, it is intended as a relative term. Thus, for example, a purified protein preparation is one in which the protein is more enriched than the protein is in its generative environment, for instance within a cell or in a biochemical reaction chamber. Preferably, a preparation of protein is purified such that the protein represents at least 50% of the total protein content of the preparation.

Recombinant: A recombinant nucleic acid is one that has a sequence that is not naturally occurring or has a sequence that is made by an artificial combination of two otherwise separated segments of sequence. This artificial combination is often accomplished by chemical synthesis or, more commonly, by the artificial manipulation of isolated segments of nucleic acids, e.g., by genetic engineering techniques. Similarly, a recombinant protein is one encoded for by a recombinant nucleic acid molecule.

Regulatory sequences or elements: These terms refer generally to a class of polynucleotide molecules (such as DNA molecules, having DNA sequences) that influence or control transcription or translation of an operably linked transcribable polynucleotide molecule, and thereby expression of genes. Included in the term are promoters, enhancers, leaders, introns, locus control regions, boundary elements/insulators, silencers, Matrix attachment regions (also referred to as scaffold attachment regions), repressor, transcriptional terminators (a.k.a. transcription termination regions), origins of replication, centromeres, and meiotic recombination hotspots. Promoters are sequences of DNA near the 5′ end of a gene that act as a binding site for RNA polymerase, and from which transcription is initiated. Enhancers are control elements that elevate the level of transcription from a promoter, usually independently of the enhancer's orientation or distance from the promoter. Locus control regions (LCRs) confer tissue-specific and temporally regulated expression to genes to which they are linked. LCRs function independently of their position in relation to the gene, but are copy-number dependent. It is believed that they function to open the nucleosome structure, so other factors can bind to the DNA. LCRs may also affect replication timing and origin usage. Insulators (also known as boundary elements) are DNA sequences that prevent the activation (or inactivation) of transcription of a gene, by blocking effects of surrounding chromatin. Silencers and repressors are control elements that suppress gene expression; they act on a gene independently of their orientation or distance from the gene. Matrix attachment regions (MARs), also known as scaffold attachment regions, are sequences within DNA that bind to the nuclear scaffold. They can affect transcription, possibly by separating chromosomes into regulatory domains. It is believed that MARs mediate higher-order, looped structures within chromosomes. Transcriptional terminators are regions within the gene vicinity that RNA polymerase is released from the template. Origins of replication are regions of the genome that, during DNA synthesis or replication phases of cell division, begin the replication process of DNA. Meiotic recombination hotspots are regions of the genome that recombine more frequently than the average during meiosis. Specific nucleotides within a regulatory region may serve multiple functions. For example, a specific nucleotide may be part of a promoter and participate in the binding of a transcriptional activator protein. Isolated regulatory elements that function in cells (for instance, in plants or plant cells) are useful for modifying plant phenotypes, for instance through genetic engineering.

RNA: A typically linear polymer of ribonucleic acid monomers, linked by phosphodiester bonds. Naturally occurring RNA molecules fall into three general classes, messenger (mRNA, which encodes proteins), ribosomal (rRNA, components of ribosomes), and transfer (tRNA, molecules responsible for transferring amino acid monomers to the ribosome during protein synthesis). Messenger RNA includes heteronuclear (hnRNA) and membrane-associated polysomal RNA (attached to the rough endoplasmic reticulum). Total RNA refers to a heterogeneous mixture of all types of RNA molecules.

Screenable Marker: A marker that confers a trait identified through observation or testing.

Selectable Marker: A marker that confers a trait that one can select for by chemical means, e.g., through the use of a selective agent (e.g., an herbicide, antibiotic, or the like). Selectable markers include but are not limited to antibiotic resistance genes, such as, kanamycin (nptII), G418, bleomycin, hygromycin, chloramphenicol, ampicillin, tetracycline, or the like. Additional selectable markers include a bar gene which codes for bialaphos resistance; a mutant EPSP synthase gene which encodes glyphosate resistance; a nitrilase gene which confers resistance to bromoxynil; a mutant acetolactate synthase gene (ALS) which confers imidazolinone or sulphonylurea resistance; or a methotrexate resistant DHFR gene. In one example, the selectable marker is AAD1.

Sequence identity: The similarity between two nucleic acid sequences, or two amino acid sequences, is expressed in terms of the similarity between the sequences, otherwise referred to as sequence identity. Sequence identity is frequently measured in terms of percentage identity (or similarity or homology); the higher the percentage, the more similar the two sequences are. Percent sequence identity is represented as the identity fraction multiplied by 100. The comparison of one or more polynucleotide or polypeptide sequences may be to a full-length polynucleotide or polypeptide sequence or a portion thereof, or to a longer polynucleotide sequence.

Methods of alignment of sequences for comparison are well known in the art. Various programs and alignment algorithms are described in: Smith and Waterman (Adv. Appl. Math. 2: 482, 1981); Needleman and Wunsch (J. Mol. Biol. 48: 443, 1970); Pearson and Lipman (PNAS. USA 85: 2444, 1988); Higgins and Sharp (Gene, 73: 237-244, 1988); Higgins and Sharp (CABIOS 5: 151-153, 1989); Corpet et al. (Nuc. Acids Res. 16: 10881-90, 1988); Huang et al. (Comp. Appls Biosci. 8: 155-65, 1992); and Pearson et al. (Methods in Molecular Biology 24: 307-31, 1994). Altschul et al. (Nature Genet., 6: 119-29, 1994) presents a detailed consideration of sequence alignment methods and homology calculations.

The alignment tools ALIGN (Myers and Miller, CABIOS 4:11-17, 1989) or LFASTA (Pearson and Lipman, 1988) may be used to perform sequence comparisons (Internet Program © 1996, W. R. Pearson and the University of Virginia, “fasta20u63” version 2.0u63, release date December 1996). ALIGN compares entire sequences against one another, while LFASTA compares regions of local similarity. These alignment tools and their respective tutorials are available on the Internet at with a web address of biology.ncsa.uiuc.edu.

Orthologs or paralogs (more generally, homologs) of a specified sequence are typically characterized by possession of greater than 75% sequence identity counted over the full-length alignment with the amino acid sequence of a specified protein (or the nucleic acid sequence of a specified nucleic acid molecule) using ALIGN set to default parameters. Sequences with even greater similarity to the reference sequences will show increasing percentage identities when assessed by this method, such as at least 80%, at least 85%, at least 90%, at least 92%, at least 95%, or at least 98% sequence identity. In such an instance, percentage identities will be essentially similar to those discussed for full-length sequence identity. An alternative indication that two nucleic acid molecules are closely related is that the two molecules hybridize to each other under stringent conditions. Stringent conditions are sequence-dependent and are different under different environmental parameters. Generally, stringent conditions are selected to be about 5° C. to 20° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. Conditions for nucleic acid hybridization and calculation of stringencies can be found in Sambrook et al. (In Molecular Cloning: A Laboratory Manual, Cold Spring Harbor, N.Y., 1989) and Tijssen (Laboratory Techniques in Biochemistry and Molecular Biology Part I, Ch. 2, Elsevier, New York, 1993). Nucleic acid molecules that hybridize under stringent conditions to a specified protein sequence will typically hybridize to a probe based on either the protein encoding sequence, an entire domain, or other selected portions of the encoding sequence under wash conditions of 0.2×SSC, 0.1% SDS at 65° C.

Nucleic acid sequences that do not show a high degree of identity may nevertheless encode similar amino acid sequences, due to the degeneracy of the genetic code. It is understood that changes in nucleic acid sequence can be made using this degeneracy to produce multiple nucleic acid sequences that each encode substantially the same protein. Substantial percent sequence identity is at least about 80% sequence identity, such as at least about 80%, at least about 85%, at least about 90%, at least about 95%, or even greater sequence identity, such as about 98% or about 99% sequence identity.

A transgenic event is produced by transformation of plant cells with a heterologous DNA construct(s), including a nucleic acid expression cassette that includes a transgene of interest, the regeneration of a population of plants resulting from the insertion of the transgene into the genome of the plant, and selection of a particular plant characterized by insertion into a particular genome location. In some embodiments of this disclosure, the transgene of interest is operable linked to a disclosed inducible promoter. An event is characterized phenotypically by the expression of the transgene(s). At the genetic level, an event is part of the genetic makeup of a plant. The term “event” also refers to progeny produced by a sexual outcross between the transformant and another variety that include the heterologous DNA. Even after repeated back-crossing to a recurrent parent, the inserted DNA and flanking DNA from the transformed parent is present in the progeny of the cross at the same chromosomal location. The term “event” also refers to DNA from the original transformant comprising the inserted DNA and flanking sequence immediately adjacent to the inserted DNA that would be expected to be transferred to a progeny that receives inserted DNA including the transgene of interest as the result of a sexual cross of one parental line that includes the inserted DNA (e.g., the original transformant and progeny resulting from selfing) and a parental line that does not contain the inserted DNA.

Transgenic plant: A plant that contains a foreign (heterologous) nucleotide sequence inserted into either its nuclear genome or organellar genome.

Transgene: A nucleic acid sequence that is inserted into a host cell or host cells by a transformation technique.

Transgenic: This term refers to a plant/fungus/cell/other entity or organism that contains recombinant genetic material not normally found in entities of this type/species (that is, heterologous genetic material) and which has been introduced into the entity in question (or into progenitors of the entity) by human manipulation. Thus, a plant that is grown from a plant cell into which recombinant DNA is introduced by transformation (a transformed plant cell) is a transgenic plant, as are all offspring of that plant that contain the introduced transgene (whether produced sexually or asexually).

Transformation: Process by which exogenous DNA enters and changes a recipient cell. It may occur under natural conditions, or artificial conditions using various methods well known in the art. Transformation may rely on any known method for the insertion of foreign nucleic acid sequences into a prokaryotic or eukaryotic host cell. Selection of the method is influenced by the host cell being transformed and may include, but is not limited to, viral infection, electroporation, lipofection, and particle bombardment.

Vector: A nucleic acid molecule as introduced into a host cell, thereby producing a transformed host cell. A vector may include nucleic acid sequences that permit it to replicate in the host cell, such as an origin of replication. A vector may also include one or more therapeutic genes and/or selectable marker genes and other genetic elements known in the art. A vector can transduce, transform or infect a cell, thereby causing the cell to express nucleic acids and/or proteins other than those native to the cell. A vector optionally includes materials to aid in achieving entry of the nucleic acid into the cell, such as a viral particle, liposome, protein coating or the like.

Suitable methods and materials for the practice or testing of this disclosure are described below. Such methods and materials are illustrative only and are not intended to be limiting. Other methods and materials similar or equivalent to those described herein can be used. For example, conventional methods well known in the art to which a disclosed invention pertains are described in various general and more specific references, including, for example, Sambrook et al., Molecular Cloning: A Laboratory Manual, 2d ed., Cold Spring Harbor Laboratory Press, 1989; Sambrook et al., Molecular Cloning: A Laboratory Manual, 3d ed., Cold Spring Harbor Press, 2001; Ausubel et al., Current Protocols in Molecular Biology, Greene Publishing Associates, 1992 (and Supplements to 2000); and Ausubel et al., Short Protocols in Molecular Biology: A Compendium of Methods from Current Protocols in Molecular Biology, 4th ed., Wiley & Sons, 1999.

### III. Description of Several Embodiments

The present disclosure describes nucleic acids, such as cDNAs, and/or mRNAs, encoding terpene synthases obtained from Selaginella moellendorffii, such as set forth in SEQ ID NOs: 1-47 or functional fragment thereof. Also provided are DNA constructs comprising the described nucleic acids encoding terpene synthases. Host cells including a disclosed S. moellendorffii nucleic acid are also provided as well as methods of producing terpenes from such host cells. In one embodiment, the terpene synthase gene confers an agronomic trait to a plant in which it is expressed, for example production of terpenes and/or pest resistance.

Also provided are transgenic plants. In one embodiment, a transgenic plant is stably transformed with a disclosed DNA construct. In some embodiments, the transgenic plant is a dicotyledon. In other embodiments, the transgenic plant is a monocotyledon. Further provided is a seed of a disclosed transgenic plant. In one embodiment, the seed comprises the disclosed DNA construct. Even further provided is a transgenic plant cell or tissue. In one embodiment, a transgenic plant cell or tissue comprises a disclosed nucleic acid encoding terpene synthases obtained from S. moellendorffii, such as set forth in SEQ ID NOs: 1-47 or functional fragment thereof. In some embodiments, the plant cell or tissue is derived from a dicotyledon. In other embodiments, the plant cell or tissue is from a monocotyledon.

Also provided are methods of producing a disclosed transgenic plant, plant cell, seed or tissue. In some embodiments, the method comprises transforming a plant cell or tissue with a disclosed DNA construct. In some embodiments, the method is a method of enhancing disease, and/or pest resistance in a plant.

Further provided are a plant cell, fruit, leaf, root, shoot, flower, seed, cutting and other reproductive material useful in sexual or asexual propagation, progeny plants inclusive of F1 hybrids, male-sterile plants and all other plants and plant products derivable from the disclosed transgenic plants.

A. Terpene Synthases

The present disclosure provides previously unrecognized terpene synthase nucleic acids, such as cDNA and mRNA, from S. moellendorffii, such as set forth in SEQ ID NOs: 1-47, which have been designated SmMTPSL 1, 2 and 4 through 48 (as SmMTPSL is a pseudogene). While a particular nucleic acid sequence has been shown for each of SmMTPSL 1, 2 and 4 through 48, it is understood that a SmMTPSL 1, 2 and 3 through 48 nucleic acid sequence includes any nucleic acid sequence redundant by virtue of the degeneracy of genetic code that encodes a SmMTPSL 1, 2 and 4 through 48 protein, or functional fragment thereof. In some embodiment, a terpene synthase from S. moellendorffii has the nucleic acid sequence as set forth on GENBANK® accession number JX413782, JX413783, JX413784, JX413785, JX413786, JX413787, JX413788, or JX413789, all of which are specifically incorporated herein in their entirety, as available Jul. 30, 2013.

Variants of the disclosed terpene synthase nucleic acids, such as cDNA and mRNA, from S. moellendorffii are also contemplated by this disclosure. Variant nucleotide sequences also include synthetically derived nucleotide sequences, such as those generated, for example, by using site-directed mutagenesis, but which when expressed still exhibit terpene synthase activity. Methods for mutagenesis and nucleotide sequence alterations are well known in the art. See, for example, Kunkel (1985) Proc. Natl. Acad. Sci. USA 52:488-492; Kunkel et al. (1987) Methods in Enzymol. 75:367-382; U.S. Pat. No. 4,873,192; Walker and Gaastra, eds. (1983) Techniques in Molecular Biology (MacMillan Publishing Company, New York) and the references cited therein. It will further be understood that amino acid sequences encoded by SmMTPSL 1, 2 and 4 through 48 nucleic acids will typically tolerate substitutions in the amino acid sequence and substantially retain biological activity. Thus, disclosed are nucleotide acids having at least 80% sequence identity to a nucleic acid sequence encoding the polypeptide that is encoded by the nucleic acid set forth as one of SEQ ID NOs: 1-47, such as at least at 80%, at least 85%, at least 90%, at least, 95% at least 96%, at least 97%, at least 98% at least 99% sequence identity or even greater. In some examples, a S. moellendorffii terpene synthase nucleic acid is at least 80% identical to the nucleic acid set forth as one of SEQ ID NOs: 1-47, such as at least at 80%, at least 85%, at least 90%, at least, 95% at least 96%, at least 97%, at least 98% at least 99% sequence identity or even greater.

To routinely identify biologically active proteins, amino acid substitutions may be based on any characteristic known in the art, including the relative similarity or differences of the amino acid side-chain substituents, for example, their hydrophobicity, hydrophilicity, charge, size, and the like. Generally, nucleotide sequence variants will encode a protein have at least 80% sequence identity to the protein encoded by a disclosed terpene synthase nucleic acid, such as at least 80%, at least 85%, at least 90%, at least, 95% at least 96%, at least 97%, at least 98% at least 99% sequence identity or even greater to the protein encoded by its respective reference terepene synthase nucleotide sequence.

In some embodiments, a disclosed terpene synthase nucleic acid encodes a functional fragment of one of SmMTPSL 1, 2 and 4 through 48 protein. Such functional fragments still exhibit terpene synthase activity. Functional fragments include proteins in which residues at the N-terminus, C-terminus and/or internal to the full length protein have been deleted. For example a deletion of less than about 50, 40, 30, 25, 20, 15, 10, 5, 4, 3, 2, or 1 amino acids from the N-terminus, C-terminus and/or internal loops can be made while maintaining the active site with minimal testing and/or experimentation to determine the activity of the resultant protein. Also disclosed are isolated proteins that have at least 80% sequence homology to the polypeptide encoded by a nucleic acid with nucleic acid sequence set forth by one of SEQ ID NOs: 1-47 or a degenerate nucleic acid, such as at least at 80%, at least 85%, at least 90%, at least, 95% at least 96%, at least 97%, at least 98% at least 99% sequence identity or even greater to the protein encoded by a nucleic acid sequence set forth by one of SEQ ID NOs: 1-47 or a degenerate nucleic acid. In some embodiments, a terpene synthase protein from S. moellendorffii includes an amino acid sequence that is at that have at least 80% sequence homology to the polypeptide set forth as one of SEQ ID NOs: 48-89 or a functional fragment thereof, such as a fragment having terpene synthase activity.

B. Expression of Terpene Synthases

The terpene synthase nucleic acids disclosed herein include recombinant DNA which is incorporated into a vector; into an autonomously replicating plasmid or virus; or into the genomic DNA of a prokaryote or eukaryote, or which exists as a separate molecule (for example, a cDNA) independent of other sequences. DNA sequences encoding S. moellendorffii terpene synthases, such as SmMTPSL 1, 2 and 4 through 48 polypeptides, can be expressed in vitro by DNA transfer into a suitable host cell. The cell may be prokaryotic or eukaryotic. The term also includes any progeny of the subject host cell. It is understood that all progeny may not be identical to the parental cell since there may be mutations that occur during replication. Methods of stable transfer, meaning that the foreign DNA is continuously maintained in the host, are known in the art. Such host cells can be used to produce terpenes. Thus, disclosed are methods for producing terpenes,

DNA sequences can be manipulated with standard procedures such as restriction enzyme digestion, fill-in with DNA polymerase, deletion by exonuclease, extension by terminal deoxynucleotide transferase, ligation of synthetic or cloned DNA sequences, site-directed sequence-alteration via single-stranded bacteriophage intermediate or with the use of specific oligonucleotides in combination with PCR. A nucleic acid encoding a S. moellendorffii terpene synthase can be cloned or amplified by in vitro methods, such as the polymerase chain reaction (PCR), the ligase chain reaction (LCR), the transcription-based amplification system (TAS), the self-sustained sequence replication system (3SR) and the Qβ replicase amplification system (QB). For example, a polynucleotide encoding the protein can be isolated by polymerase chain reaction of cDNA using primers based on the DNA sequence of the molecule. A wide variety of cloning and in vitro amplification methodologies are well known to persons skilled in the art. PCR methods are described in, for example, U.S. Pat. No. 4,683,195; Mullis et al., Cold Spring Harbor Symp. Quant. Biol. 51:263, 1987; and Erlich, ed., PCR Technology, (Stockton Press, NY, 1989). Polynucleotides also can be isolated by screening genomic or cDNA libraries with probes selected from the sequences of the desired polynucleotide under stringent hybridization conditions.

Terpene synthase nucleic acids, such as cDNA sequences encoding mMTPSL 1, 2 and 4 through 48 polypeptides, can be operatively linked to expression control sequences. An expression control sequence operatively linked to a coding sequence is ligated such that expression of the coding sequence is achieved under conditions compatible with the expression control sequences. The expression control sequences include, but are not limited to appropriate promoters, enhancers, transcription terminators, a start codon (for instance, ATG) in front of a protein-encoding gene, splicing signal for introns, maintenance of the correct reading frame of that gene to permit proper translation of mRNA, and stop codons.

Transformation of a host cell with recombinant DNA may be carried out by conventional techniques as are well known to those skilled in the art. Where the host is prokaryotic, such as E. coli, competent cells, which are capable of DNA uptake can be prepared from cells harvested after exponential growth phase and subsequently treated by the CaCl₂ method using procedures well known in the art. Alternatively, MgCl₂, or RbCl can be used. Transformation can also be performed after forming a protoplast of the host cell if desired, or by electroporation.

When the host is a eukaryote, such methods of transfection of DNA as calcium phosphate coprecipitates, conventional mechanical procedures such as microinjection, electroporation, insertion of a plasmid encased in liposomes, or virus vectors may be used. Eukaryotic cells can also be cotransformed with a second foreign DNA molecule encoding a selectable phenotype, such as the herpes simplex thymidine kinase gene. Another method is to use a eukaryotic viral vector, such as simian virus 40 (SV40) or bovine papilloma virus, to transiently infect or transform eukaryotic cells and express the protein (see for example, Eukaryotic Viral Vectors, Cold Spring Harbor Laboratory, Gluzman ed., 1982).

The expression and purification of any of S. moellendorffii terpene synthase proteins, by standard laboratory techniques, is now enabled. Fragments amplified as described herein can be cloned into standard cloning vectors and expressed in commonly used expression systems consisting of a cloning vector and a cell system in which the vector is replicated and expressed. Purified proteins may be used for functional analyses. Partial or full-length cDNA sequences, which encode for the protein, may be ligated into bacterial expression vectors. Methods for expressing large amounts of protein from a cloned gene introduced into E. coli may be utilized for the purification, localization and functional analysis of proteins and terpenes.

Intact native protein may also be produced in E. coli in large amounts for functional studies. Standard prokaryotic cloning vectors may also be used, for example, pBR322, pUC18, or pUC19 as described in Sambrook et al. (Molecular Cloning: A Laboratory Manual, 2^{nd} ed., vol. 1-3, Cold Spring Harbor, N.Y. 1989). Nucleic acids of terpene synthase nucleic acids, such as cDNA sequences encoding mMTPSL 1, 2 and 4 through 48 polypeptides may be cloned into such vectors, which may then be transformed into bacteria such as E. coli, which may then be cultured so as to express the protein of interest. Other prokaryotic expression systems include, for instance, the arabinose-induced pBAD expression system that allows tightly controlled regulation of expression, the IPTG-induced pRSET system that facilitates rapid purification of recombinant proteins and the IPTG-induced pSE402 system that has been constructed for optimal translation of eukaryotic genes. These three systems are available commercially from INVITROGEN™ and, when used according to the manufacturer's instructions, allow routine expression and purification of proteins.

Methods and plasmid vectors for producing fusion proteins and intact native proteins in bacteria are described in Sambrook et al. (Molecular Cloning: A Laboratory Manual, Cold Spring Harbor, N.Y., 1989, Chapter 17). Such fusion proteins may be made in large amounts and are easy to purify. Proteins can be produced in bacteria by placing a strong, regulated promoter and an efficient ribosome binding site upstream of the cloned gene. If low levels of protein are produced, additional steps may be taken to increase protein production; if high levels of protein are produced, purification is relatively easy. Suitable methods are presented in Sambrook et al. (Molecular Cloning: A Laboratory Manual, Cold Spring Harbor, N.Y., 1989) and are well known in the art. Often, proteins expressed at high levels are found in insoluble inclusion bodies. Methods for extracting proteins from these aggregates are described by Sambrook et al. (Molecular Cloning: A Laboratory Manual, Cold Spring Harbor, N.Y., 1989, Chapter 17).

A number of viral vectors have been constructed, that can be used to express the disclosed antigens, including polyoma, i.e., SV40 (Madzak et al., 1992, J. Gen. Virol., 73:15331536), adenovirus (Berkner, 1992, Cur. Top. Microbiol. Immunol., 158:39-6; Berliner et al., 1988, Bio Techniques, 6:616-629; Gorziglia et al., 1992, J. Virol., 66:4407-4412; Quantin et al., 1992, Proc. Natl. Acad. Sci. USA, 89:2581-2584; Rosenfeld et al., 1992, Cell, 68:143-155; Wilkinson et al., 1992, Nucl. Acids Res., 20:2233-2239; Stratford-Perricaudet et al., 1990, Hum. Gene Ther., 1:241-256), vaccinia virus (Mackett et al., 1992, Biotechnology, 24:495-499), adeno-associated virus (Muzyczka, 1992, Curr. Top. Microbiol. Immunol., 158:91-123; On et al., 1990, Gene, 89:279-282), herpes viruses including HSV and EBV (Margolskee, 1992, Curr. Top. Microbiol. Immunol., 158:67-90; Johnson et al., 1992, J. Virol., 66:29522965; Fink et al., 1992, Hum. Gene Ther. 3:11-19; Breakfield et al., 1987, Mol. Neurobiol., 1:337-371; Fresse et al., 1990, Biochem. Pharmacol., 40:2189-2199), Sindbis viruses (H. Herweijer et al., 1995, Human Gene Therapy 6:1161-1167; U.S. Pat. Nos. 5,091,309 and 5,2217,879), alphaviruses (S. Schlesinger, 1993, Trends Biotechnol. 11:18-22; I. Frolov et al., 1996, Proc. Natl. Acad. Sci. USA 93:11371-11377) and retroviruses of avian (Brandyopadhyay et al., 1984, Mol. Cell. Biol., 4:749-754; Petropouplos et al., 1992, J. Virol., 66:3391-3397), murine (Miller, 1992, Curr. Top. Microbiol. Immunol., 158:1-24; Miller et al., 1985, Mol. Cell. Biol., 5:431-437; Sorge et al., 1984, Mol. Cell. Biol., 4:1730-1737; Mann et al., 1985, J. Virol., 54:401-407), and human origin (Page et al., 1990, J. Virol., 64:5370-5276; Buchschalcher et al., 1992, J. Virol., 66:2731-2739). Baculovirus (Autographa californica multinuclear polyhedrosis virus; AcMNPV) vectors are also known in the art, and may be obtained from commercial sources (such as PharMingen, San Diego, Calif.; Protein Sciences Corp., Meriden, Conn.; Stratagene, La Jolla, Calif.).

Various yeast strains and yeast-derived vectors are commonly used for expressing and purifying proteins, for example, Pichia pastoris expression systems are available from INVITROGEN™ (Carlsbad, Calif.). Such systems include suitable Pichia pastoris strains, vectors, reagents, transformants, sequencing primers and media.

Non-yeast eukaryotic vectors can also be used for expression of the S. moellendorffii terpene synthases such as mMTPSL 1, 2 and 4 through 48 polypeptides. Examples of such systems are the well known Baculovirus system, the Ecdysone-inducible mammalian expression system that uses regulatory elements from Drosophila melanogaster to allow control of gene expression, and the Sindbis viral expression system that allows high level expression in a variety of mammalian cell lines. These expression systems are available from INVITROGEN™.

In addition, some vectors contain selectable markers such as the gpt (Mulligan and Berg, Proc. Natl. Acad. Sci. USA 78:2072-6, 1981) or neo (Southern and Berg, J. Mol. Appl. Genet. 1:327-41, 1982) bacterial genes. These selectable markers permit selection of transfected cells that exhibit stable, long-term expression of the vectors (and therefore the cDNA). The vectors can be maintained in the cells as episomal, freely replicating entities by using regulatory elements of viruses such as papilloma (Sarver et al., Mol. Cell. Biol. 1:486, 1981) or Epstein-Barr (Sugden et al., Mol. Cell. Biol. 5:410, 1985). Alternatively, one can also produce cell lines that have integrated the vector into genomic DNA. Both of these types of cell lines produce the gene product on a continuous basis. One can also produce cell lines that have amplified the number of copies of the vector (and therefore of the cDNA as well) to create cell lines that can produce high levels of the gene product (Alt et al., J. Biol. Chem. 253:1357, 1978).

The transfer of DNA into eukaryotic cells is now a conventional technique. The vectors are introduced into the recipient cells as pure DNA (transfection) by, for example, precipitation with calcium phosphate (Graham and vander Eb, 1973, Virology 52:466) or strontium phosphate (Brash et al., Mol. Cell. Biol. 7:2013, 1987), electroporation (Neumann et al., EMBO J. 1:841, 1982), lipofection (Felgner et al., Proc. Natl. Acad. Sci. USA 84:7413, 1987), DEAE dextran (McCuthan et al., J. Natl. Cancer Inst. 41:351, 1968), microinjection (Mueller et al., Cell 15:579, 1978), protoplast fusion (Schather, Proc. Natl. Acad. Sci. USA 77:2163-7, 1980), or pellet guns (Klein et al, Nature 327:70., 1987). Alternatively, the cDNA can be introduced by infection with virus vectors. Systems are developed that use, for example, retroviruses (Bernstein et al., Gen. Engrg. 7:235, 1985), adenoviruses (Ahmad et al., J. Virol. 57:267, 1986), or Herpes virus (Spaete et al., Cell 30:295, 1982).

Where appropriate, the nucleotide sequences whose expression is desired may be optimized for increased expression in host cell. That is, these nucleotide sequences can be synthesized using plant preferred codons for improved expression.

C. Transgenics

The nucleotide sequences for the disclosed S. moellendorffii terpene synthases, such a nucleic acid sequence encoding of one ofmMTPSL 1, 2 and 4 through 48 (such as having at least 80% sequence homologs to the nucleic acid sequence set forth by one of SEQ ID NOs: 1-47 or a degenerate nucleic acid) or functional fragment thereof, are useful in the genetic manipulation plant cells to confer terpene synthesis when operably linked with a promoter, such as an indictable or constitutive promoter. In this manner, the nucleotide sequences for the S. moellendorffii terpene synthases are provided in expression cassettes for expression in the plant of interest. Such expression cassettes will typically comprise a transcriptional initiation region comprising a promoter nucleotide sequence operably linked to one or more of the disclosed terpene synthase nucleic acids or variants thereof. Such an expression cassette can be provided with a plurality of restriction sites for insertion of the nucleotide sequence to be under the transcriptional regulation of the regulatory regions. The expression cassette may additionally contain selectable marker genes or sequences. The expression cassettes of this disclosure can be part of and an expression vector, such as a plasmid.

In some embodiments, the transcriptional cassette will include in the 5′-to-3′ direction of transcription, a transcriptional and translational initiation region, a terpene synthase nucleic acid, such a nucleic acid sequence encoding of one of mMTPSL 1, 2 and 4 through 48 (such as having at least 80% sequence identity to the nucleic acid sequence set forth by one of SEQ ID NOs: 1-47 or a degenerate nucleic acid) or functional fragment thereof, and a transcriptional and translational termination region functional in plant cells. The termination region may be native with the transcriptional initiation region, may be native with the S. moellendorffii terpene synthase, or may be derived from another source. Convenient termination regions are available from the Ti-plasmid of A. tumefaciens, such as the octopine synthase and nopaline synthase termination regions. See also, Guerineau et al., Mol. Gen. Genet. 262:141-144, 1991; Proudfoot Cell 64:671-674, 1991; Sanfacon et al., Genes Dev. 5:141-149, 1991; Mogen et al., Plant Cell 2:1261-1272, 1990; Munroe et al., Gene 91:151-158, 1990; Ballas et al., Nucleic Acids Res. 17:7891-7903, 1989; Joshi et al., Nucleic Acid Res. 15:9627-9639, 1987.

An expression cassette including a disclosed S. moellendorffii terpene synthase operably linked to a promoter sequence may also contain at least additional nucleotide sequence for a gene to be cotransformed into the organism. Alternatively, the additional sequence(s) can be provided on another expression cassette.

Where appropriate, the nucleotide sequences whose expression is desired may be optimized for increased expression in the transformed plant. That is, these nucleotide sequences can be synthesized using plant preferred codons for improved expression. Methods are available in the art for synthesizing plant-preferred nucleotide sequences. See, for example, U.S. Pat. Nos. 5,380,831 and 5,436,391, and Murray et al., Nucleic Acids Res. 17:477-498, 1989.

Additional sequence modifications are known to enhance gene expression in a cellular host. These include elimination of sequences encoding spurious polyadenylation signals, exon-intron splice site signals, transposon-like repeats, and other such well-characterized sequences that may be deleterious to gene expression. The G-C content of the heterologous nucleotide sequence may be adjusted to levels average for a given cellular host, as calculated by reference to known genes expressed in the host cell. When possible, the sequence is modified to avoid predicted hairpin secondary mRNA structures.

The expression cassettes may additionally contain 5′ leader sequences in the expression cassette construct. Such leader sequences can act to enhance translation. Translation leaders are known in the art and include: picornavirus leaders, for example, EMCV leader (Encephalomyocarditis 5′ noncoding region) (Elroy-Stein et al., Proc. Nat. Acad. Sci. USA 86:6126-6130, 1989); potyvirus leaders, for example, TEV leader (Tobacco Etch Virus); MDMV leader (Maize Dwarf Mosaic Virus); human immunoglobulin heavy-chain binding protein (BiP) (Macejak and Sarnow Nature 353:90-94, 1991); untranslated leader from the coat protein mRNA of alfalfa mosaic virus (AMV RNA 4) (Jobling and Gehrke Nature 325:622-625, 1987); tobacco mosaic virus leader (TMV) (Gallie et al. Molecular Biology of RNA, pages 237-256, 1989; and maize chlorotic mottle virus leader (MCMV) (Lommel et al., Virology 81:382-385, 1991). See also Della-Cioppa et al., Plant Physiology 84:965-968, 1987. Other methods known to enhance translation and/or mRNA stability can also be utilized, for example, introns, and the like.

In those instances where it is desirable to have the expressed product of the S. moellendorffii terpene synthase directed to a particular organelle, such as the chloroplast or mitochondrion, or secreted at the cell's surface or extracellularly, the expression cassette may further comprise a coding sequence for a transit peptide. Such transit peptides are well known in the art and include, but are not limited to, the transit peptide for the acyl carrier protein, the small subunit of RUBISCO, plant EPSP synthase, and the like.

In preparing the expression cassette, the various DNA fragments may be manipulated by methods known in the art, so as to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites, or the like. For this purpose, in vitro mutagenesis, primer repair, restriction, annealing, resubstitutions, for example, transitions and transversions, may be involved.

The expression cassettes may include reporter genes or selectable marker genes. Examples of suitable reporter genes known in the art can be found in, for example, Jefferson et al. in Plant Molecular Biology Manual, ed. Gelvin et al. (Kluwer Academic Publishers), pp. 1-33, 1991; DeWet et al., Mol. Cell. Biol. 7:725-737, 1987; Goff et al., EMBO J. 9:2517-2522, 1990; and Kain et al., BioTechniques 19:650-655, 1995; and Chiu et al., Current Biology 6:325-330, 1996. Selectable marker genes for selection of transformed cells or tissues can include genes that confer antibiotic resistance or resistance to herbicides. Examples of suitable selectable marker genes include, but are not limited to, genes encoding resistance to chloramphenicol (Herrera Estrella et al., EMBO J. 2:987-992, 1983); methotrexate (Herrera Estrella et al., Nature 303:209-213, 1983; Meijer et al., Plant Mol. Biol. 16:807-820, 1991); hygromycin (Waldron et al., Plant Mol. Biol. 5:103-108, 1985; Zhijian et al., Plant Science 108:219-227, 1995); streptomycin (Jones et al., Mol. Gen. Genet. 210:86-91, 1987); spectinomycin (Bretagne-Sagnard et al., Transgenic Res. 5:131-137, 1996); bleomycin (Hille et al., Plant Mol. Biol. 7:171-176, 1990); sulfonamide (Guerineau et al., Plant Mol. Biol. 15:127-136, 1990); bromoxynil (Stalker et al., Science 242:419-423, 1988); glyphosate (Shaw et al., Science 233:478-481, 1986); and phosphinothricin (DeBlock et al., EMBO J. 6:2513-2518, 1987).

Other genes that could serve utility in the recovery of transgenic events but might not be required in the final product would include, but are not limited to, such examples as GUS (b-glucoronidase; Jefferson Plant Mol. Biol. Rep. 5:387, 1987), GFP and other related fluorescent proteins, and luciferase.

An expression cassette including a disclosed a nucleic acid sequence encoding of one of mMTPSL 1, 2 and 4 through 48 (such as having at least 80% sequence identity to the nucleic acid sequence set forth by one of SEQ ID NOs: 1-47 or a degenerate nucleic acid) or functional fragment thereof, operably linked to promoter and optionally other heterologous nucleic acids can be used to transform any plant or part thereof, such as a plant cell, for example as a vector, such as a plasmid. In this manner, genetically modified plants, plant cells, plant tissue, seed, and the like can be obtained. Such methods, include introducing into a plant, such a nucleic acid sequence encoding of one of mMTPSL 1, 2 and 4 through 48 (such as having at least 80% sequence identity to the nucleic acid sequence set forth by one of SEQ ID NOs: 1-47 or a degenerate nucleic acid) or functional fragment thereof, operably linked to promoter and optionally other heterologous nucleic acids. Also disclosed are methods increasing terpene production in a plant. Such methods, include introducing into a plant, such a nucleic acid sequence encoding of one of SmMTPSL 1, 2 and 4 through 48 (such as having at least 80% sequence identity to the nucleic acid sequence set forth by one of SEQ ID NOs: 1-47 or a degenerate nucleic acid) or functional fragment thereof, operably linked to promoter and optionally other heterologous nucleic acids, thereby increasing terpene production in the plant. The plant can be transiently or stably transformed. Terpene production can be determined relative to a relevant control plant, such as a plant that does not express a terpene synthase polypeptide disclosed herein, a plant that has not been transformed with a nucleic acid encoding a terpene synthase polypeptide as disclosed herein, a plant that is transformed with an irrelevant nucleic acid, and the like. The control plant is generally matched for species, variety, age, and the like and is subjected to the same growing conditions, for example temperature, soil, sunlight, pH, water, and the like. The selection of a suitable control plant is routine for those skilled in the art.

Transformation protocols as well as protocols for introducing nucleotide sequences into plants may vary depending on the type of plant or plant cell, for example, monocot or dicot, targeted for transformation. Suitable methods of introducing nucleotide sequences into plant cells and subsequent insertion into the plant genome include microinjection (Crossway et al., Biotechniques 4:320-334, 1986), electroporation (Riggs et al., Proc. Natl. Acad. Sci. USA 53:5602-5606, 1986), Agrobacterium-mediated transformation (U.S. Pat. No. 5,563,055), direct gene transfer (Paszkowski et al., EMBO J. 3:2717-2722, 1984), and ballistic particle acceleration (see, for example, U.S. Pat. No. 4,945,050; Tomes et al. “Direct DNA Transfer into Intact Plant Cells via Microprojectile Bombardment,” in Plant Cell, Tissue, and Organ Culture: Fundamental Methods, ed. Gamborg and Phillips (Springer-Verlag, Berlin), 1995; and McCabe et al., Biotechnology 5:923-926, 1988). Also see Weissinger et al., Ann. Rev. Genet. 22:421-477, 1988; Sanford et al., Paniculate Science and Technology 5:27-37, 1987; Christou et al., Plant Physiol 57:671-674, 1988; McCabe et al., Bio/Technology 5:923-926, 1988; Finer and McMullen, In Vitro Cell Dev. Biol. 27P:175-182, 1991; Singh et al., Theor. Appl Genet. 95:319-324, 1998; Datta et al., Biotechnology 5:736-740, 1990; Klein et al., Proc. Natl. Acad. Sci. USA 55:4305-4309, 1988; Klein et al., Biotechnology 5:559-563, 1988; U.S. Pat. Nos. 5,240,855, 5,322,783 and 5,324,646; Klein et al., Plant Physiol 97:440-444, 1988; Fromm et al. Biotechnology 5:833-839, 1990; Hooykaas-Van Slogteren et al., Nature 377:763-764, 1984; Bytebier et al., Proc. Natl. Acad. Sci. USA 54:5345-5349, 1987; De Wet et al. (1985) in The Experimental Manipulation of Ovule Tissues, ed. Chapman et al. (Longman, New York), pp. 197-209; Kaeppler et al., Plant Cell Reports 9:415-418, 1990; Kaeppler et al., Theor. Appl. Genet. 54:560-566, 1992; D'Halluin et al., Plant Cell 4:1495-1505 1992; Li et al., Plant Cell Reports 72:250-255, 1993; Christou and Ford Annals of Botany 75:407-413, 1995; Osjoda et al., Nature Biotechnology 74:745-750, 1996; and the like. “Introducing” in the context of a plant cell, plant tissue, plant part and/or plant means contacting a nucleic acid molecule with the plant cell, plant tissue, plant part, and/or plant in such a manner that the nucleic acid molecule gains access to the interior of the plant cell or a cell of the plant tissue, plant part or plant. Where more than one nucleic acid molecule is to be introduced, these nucleic acid molecules can be assembled as part of a single polynucleotide or nucleic acid construct, or as separate polynucleotide or nucleic acid constructs, and can be located on the same or different nucleic acid constructs. Accordingly, these polynucleotides can be introduced into plant cells in a single transformation event, in separate transformation events, or, for example as part of a breeding protocol.

The cells that have been transformed may be grown into plants in accordance with conventional ways. See, for example, McCormick et al. Plant Cell Reports 5:81-84, 1986. These plants may then be grown, and either pollinated with the same transformed strain or different strains, and the resulting hybrid having expression of the desired phenotypic characteristic identified. Two or more generations may be grown to ensure that expression of the desired phenotypic characteristic is stably maintained and inherited and then seeds harvested to ensure expression of the desired phenotypic characteristic has been achieved.

The pest resistance genes disclosed herein, such as a nucleic acid sequence encoding of one of mMTPSL 1, 2 and 4 through 48 (such as having at least 80% sequence identity to the nucleic acid sequence set forth by one of SEQ ID NOs: 1-47 or a degenerate nucleic acid) or active variant and fragments thereof may be used for transformation of any plant species, including, but not limited to, monocots and dicots. Examples of plant species of interest include, but are not limited to, corn (Zea mays), Brassica sp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana), sunflower (Helianthus annuus), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oats, barley, vegetables, ornamentals, and conifers.

Vegetables include tomatoes (Lycopersicon esculentum), lettuce (e.g., Lactuca sativa), green beans (Phaseolus vulgaris), lima beans (Phaseolus limensis), peas (Lathyrus spp.), and members of the genus Cucumis such as cucumber (C. sativus), cantaloupe (C. cantalupensis), and musk melon (C. melo).

Ornamentals include azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia pulcherrima), and chrysanthemum.

Conifers that may be employed in practicing the present invention include, for example, pines such as loblolly pine (Pinus taeda), slash pine (Pinus elliotii), ponderosa pine (Pinus ponderosa), lodgepole pine (Pinus contorta), and Monterey pine (Pinus radiata); Douglas-fir (Pseudotsuga menziesii); Western hemlock (Tsuga canadensis); Sitka spruce (Picea glauca); redwood (Sequoia sempervirens); true firs such as silver fir (Abies amabilis) and balsam fir (Abies balsamea); and cedars such as Western red cedar (Thuja plicata) and Alaska yellow-cedar (Chamaecyparis nootkatensis), and Poplar and Eucalyptus.

In specific embodiments, plants of the present disclosure are crop plants (for example, corn, alfalfa, sunflower, Brassica, soybean, cotton, safflower, peanut, sorghum, wheat, millet, tobacco, etc.). In other embodiments, corn and soybean plants are optimal, and in yet other embodiments soybean plants are optimal. Other plants of interest include grain plants that provide seeds of interest, oilseed plants, and leguminous plants. Seeds of interest include grain seeds, such as corn, wheat, barley, rice, sorghum, rye, etc. Oil-seed plants include cotton, soybean, safflower, sunflower, Brassica, maize, alfalfa, palm, coconut, etc. Leguminous plants include beans and peas. Beans include guar, locust bean, fenugreek, soybean, garden beans, cowpea, mungbean, lima bean, fava bean, lentils, chickpea, etc.

In some embodiments, the polynucleotides comprising disclosed pest resistance gene are engineered into a molecular stack. Thus, the various plants, plant cells and seeds disclosed herein can further comprise one or more traits of interest, and in more specific embodiments, the plant, plant part or plant cell is stacked with any combination of polynucleotide sequences of interest in order to create plants with a desired combination of traits. As used herein, the term “stacked” includes having the multiple traits present in the same plant.

These stacked combinations can be created by any method including, but not limited to, breeding plants by any conventional methodology, or genetic transformation. If the sequences are stacked by genetically transforming the plants, the polynucleotide sequences of interest can be combined at any time and in any order. The traits can be introduced simultaneously in a co-transformation protocol with the polynucleotides of interest provided by any combination of transformation cassettes. For example, if two sequences will be introduced, the two sequences can be contained in separate transformation cassettes (trans) or contained on the same transformation cassette (cis). Expression of the sequences can be driven by the same promoter or by different promoters. In certain cases, it may be desirable to introduce a transformation cassette that will suppress the expression of the polynucleotide of interest. This may be combined with any combination of other suppression cassettes or overexpression cassettes to generate the desired combination of traits in the plant. It is further recognized that polynucleotide sequences can be stacked at a desired genomic location using a site-specific recombination system. See, for example, WO99/25821, WO99/25854, WO99/25840, WO99/25855, and WO99/25853.

The transformed plants may be analyzed for the presence of the gene(s) of interest and the expression level. Numerous methods are available to those of ordinary skill in the art for the analysis of transformed plants. For example, methods for plant analysis include Southern and northern blot analysis, PCR-based (or other nucleic acid amplification-based) approaches, biochemical analyses, phenotypic screening methods, field evaluations, and immunodiagnostic assays (e.g., for the detection, localization, and/or quantification of proteins).

### EXAMPLES

### Example 1

### Identification of Two Distinct Types of TPSs in the S. moellendorffii Genome

A thorough search of the S. moellendorffii genome sequence led to the identification of 66 TPS gene models (Table 1). Based on the phylogenetic analysis of S. moellendorffii TPSs and TPSs from other plants, bacteria, and fungi, these 66 TPSs can be divided into two groups, which we designated S. moellendorffii TPS proteins (SmTPSs) and S. moellendorffii microbial TPS-like proteins (SmMTPSLs). The SmTPS group consists of 18 members. SmTPSs are closely related to typical plant TPSs (FIG. 1). However, the protein sequences of the SmMTPSLs, a group that contains 48 members, are more similar to microbial TPSs than SmTPSs and other plant TPSs (FIG. 1).

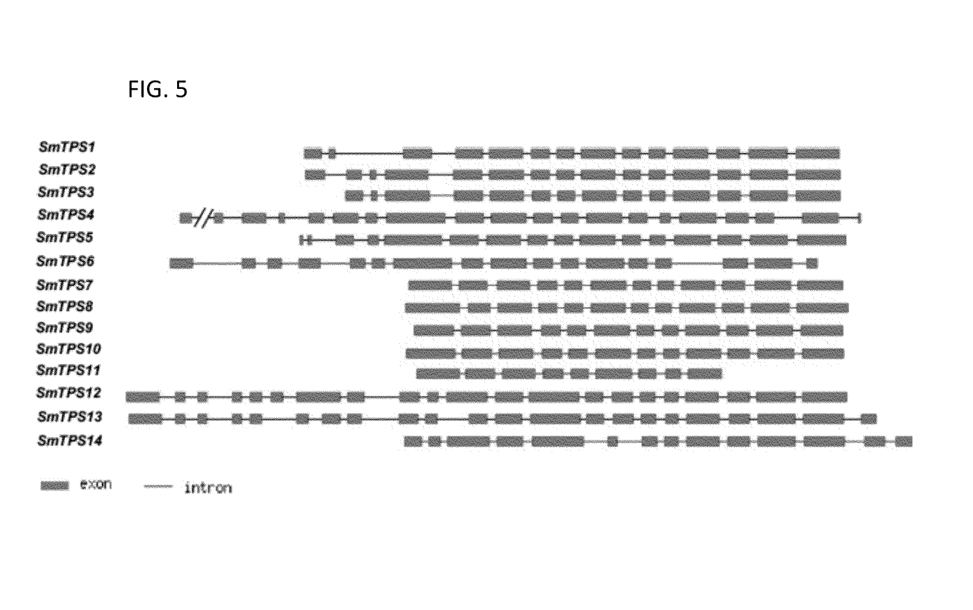

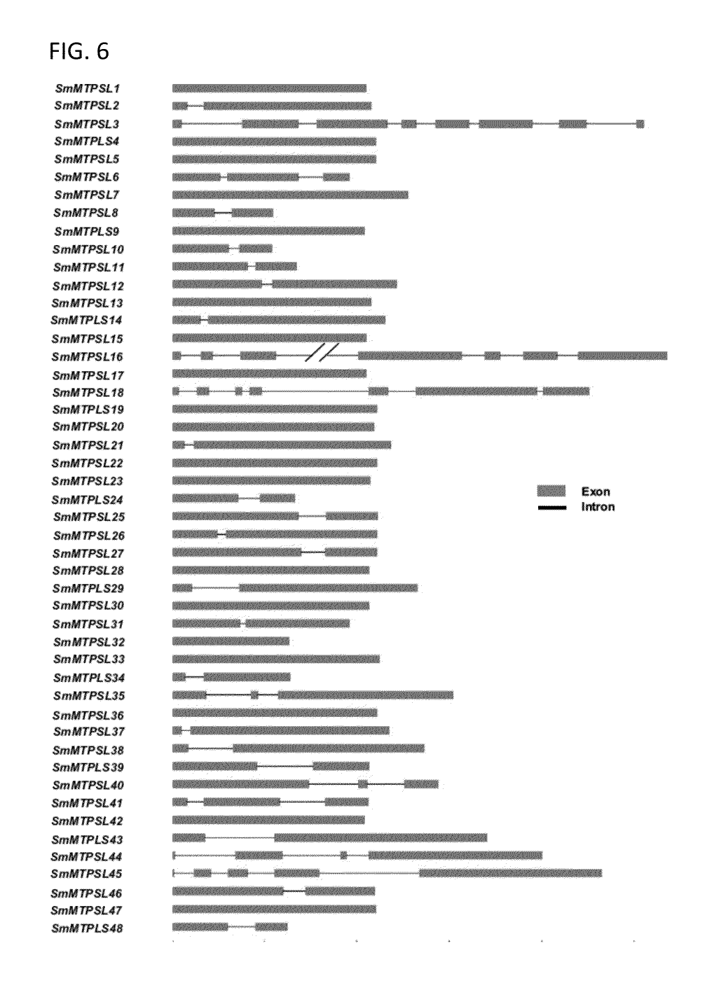

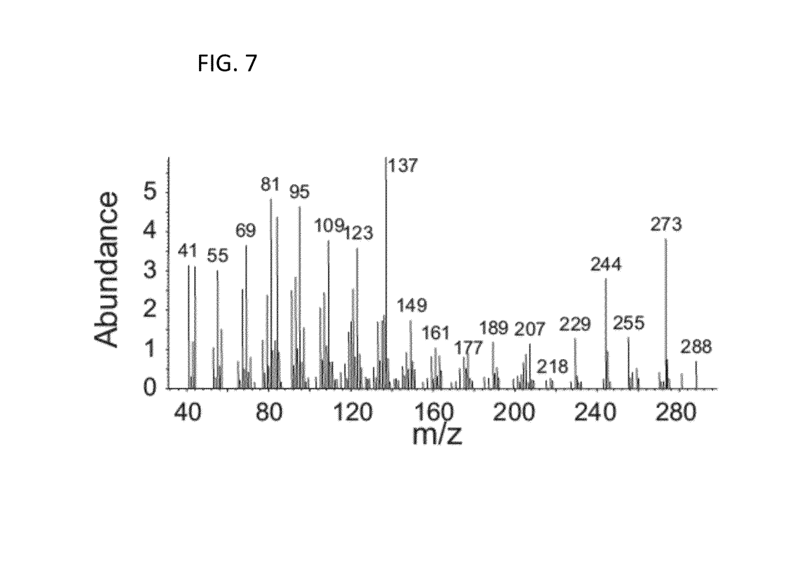

Differences between these two groups of S. moellendorffii TPSs are also evident in their gene structures and protein structures. The 14 putative full-length SmTPSs contain 11-14 introns and encode proteins of ˜800 aa in length (FIG. 5). In contrast, the SmMTPSL genes encode proteins of ˜350 aa in length and contain principally zero or one intron (FIG. 6). Furthermore, the proteins of the two S. moellendorffii TPS groups also exhibit differences in structure. As shown recently, TPS architecture is modular in nature and consists of one, two, or three separate domains, which are termed α, β, and γ (Köksal et al. Nature 469:116-120, 2011). Typical seed plant diterpene synthases are composed of three domains in the order γ, β, and α (as exemplified by taxadiene synthase from the Pacific yew, Taxus brevifolia) (Köksal et al.), and typical seed plant monoterpene and sesquiterpene (and some diterpene) synthases are composed of two domains in the order β and α (represented by 5-epi-aristolochene synthase from tobacco) (Starks et al. Science 277:1815-1820, 1997). In contrast, many microbial TPSs, such as pentalenene synthase from the bacterium Streptomyces UC5319 (Lesburg et al., Science 277:1820-1824, 1997) and trichodiene synthase from fungus Fusarium sporotrichioides (Rynkiewicz et al., Proc Natl Acad Sci USA 98:13543-13548, 2001), contain only an α-domain. In S. moellendorffii, the SmTPSs have the structure of γ/β/α, whereas the SmMTPSLs have only the α-domain. Thus, both phylogenetic analysis and gene/protein structure analysis support the conclusion that SmMTPSLs are close relatives of microbial TPSs and only distantly related to SmTPSs and other plant TPSs.

### Example 2

### Representative SmTPSs Encode Diterpene Synthases

To learn more about the two groups of S. moellendorffii TPSs, their biochemical properties were investigated. Among the 18 SmTPSs, 2 SmTPSs (SmTPS7 and SmTPS4) have been recently characterized, both of which encode bifunctional diterpene synthases. Like PpCPS/KS from the moss, SmTPS7 and SmTPS4, which have been previously named as SmCPSKSL1 and SmMDS, respectively, first catalyze the formation of CPP using GGPP as substrate. These enzymes then convert CPP to X-7,13E-dien-15-ol (Mafu et al., ChemBioChem 12:1984-1987, 2011) and miltiradiene (Sugai et al., J Biol Chem 286:42840-42847, 2011), respectively. To gain additional information about the catalytic functions of SmTPSs in this study, we chose to study SmTPS9 and SmTPS10, both of which contain only the DXDD and not the DDXXD motif, indicative of monofunctional diterpene synthases using GGPP as the direct substrate (Hayashi et al., FEBS Lett 580:6175-6181, 2006). Full-length cDNAs for SmTPS9 and SmTPS10 were isolated and expressed in Escherichia coli for the production of recombinant proteins. Both the E. coli-expressed SmTPS9 and -SmTPS10 recombinant proteins showed monofunctional diterpene synthase activity, converting GGPP to copalyl diphosphate (FIG. 2).

### Example 3

### Representative SmMTPSLs Encode Monoterpene and Sesquiterpene Synthases

To determine whether the SmMTPSL genes encode functional enzymes and if so, whether they have similar activities to SmTPSs or very different ones, we selected a group of SmMTPSLs for biochemical characterization using in vitro enzyme assays. Full-length cDNAs for six SmMTPSL genes (SmMTPSL1, SmMTPSL13, SmMTPSL17, SmMTPSL22, SmMTPSL26, and SmMTPSL30) were isolated and expressed in E. coli for the production of recombinant proteins. The SmMTPSL1, SmMTPSL17, SmMTPSL22, and SmMTPSL26 recombinant proteins all showed sesquiterpene synthase activity in vitro with farnesyl diphosphate as substrate (FIG. 3 and FIG. 8). SmMTPSL22 catalyzed the formation of nerolidol as a single product, whereas the other three SmMTPSLs each produced multiple sesquiterpenes, which is typical of many plant sesquiterpene synthases (Degenhardt et al., Phytochemistry 70:1621-1637). SmMTPSL1 produced six sesquiterpenes, and SmMTPSL17 produced eight sesquiterpenes, with five of the products of SmMTPSL1 also produced by SmMTPSL17. In contrast, SmMTPSL26 produced three sesquiterpene products that were specific to this enzyme. SmMTPSL22 also showed monoterpene synthase activity with geranyl diphosphate, catalyzing the formation of linalool as the major product (FIG. 3 and FIG. 8). SmMTPSL1, SmMTPSL17, SmMTPSL22, and SmMTPSL26 did not show diterpene synthase activity, and SmMTPSL13 and SmMTPSL30 did not show any TPS activity.

### Example 4

### Emission of Volatile Terpenes from Stressed S. moellendorffii Plants

To obtain information on whether the biochemical activities of SmMTPSLs are biologically relevant, we analyzed the volatiles emitted from S. moellendorffii plants using headspace collection combined with GC-MS. Both untreated S. moellendorffii plants and plants treated with alamethicin, a fungal antibiotic that elicits defense reactions (Engelberth et al., Plant Physiol 125:369-377, 2001), were subject to analysis. Untreated plants emitted no terpenes, but a number of terpenes were detected from alamethicin-treated plants (FIG. 4), including the monoterpene linalool and the sesquiterpenes β-elemene, germacrene D, β-sesquiphellandrene, and nerolidol (FIG. 4).

### Example 5

### Materials and Methods for Examples 1-4

Sequence Search and Analysis. TPSs in S. moellendorffii and 16 other plant species (Table 3) were identified from their genome sequences using two Pfam models PF01397 and PF03936, which correspond to the two conserved domains localized at the N and C termini of known TPSs, respectively. TPSs from bacteria and fungi were identified using the Pfam model PF03936. Phylogenetic trees were reconstructed using TPSs identified from S. moellendorffii, TPSs identified from the 16 plant genomes, 28 known TPSs from gymnosperms, and TPSs identified from bacteria and fungi. Maximum likelihood phylogenies were built using PhyML v3.0 and visualized using FigTree version 1.3.1.

The annotated proteome of S. moellendorffii (version 1.0, FilteredModels3) was searched with two Pfam models PF01397 and PF03936, which correspond to the conserved domains localized at the N and C termini of known terpene synthases (TPSs), respectively, using the hmmsearch command in the HMMER package (1). The significant hits were selected with an E value≦1e-2 as the cutoff and confirmed by InterProScan (available on the world wide web at ebi.ac.uk/Tools/pfa/iprscan). When PF01397 was used as the query, 18 TPS sequences were identified, which we designated as S. moellendorffii TPSs (SmTPSs). In these SmTPSs, there were 14 putative full-length SmTPSs containing both PF01397 and PF03936 domains. The 48 TPSs from S. moellendorffii containing only the PF03936 domain represents a type of TPSs that has not been identified in plants previously and were designated as S. moellendorffii microbial TPS-like proteins (SmMTPSLs) (Table S1).

Identification of TPSs from Other Sequenced Plant Genomes. The annotated protein sequences of 16 sequenced plant genomes (Table 3) were downloaded from various sources. These proteomes were individually searched using the same method described in searching the proteome of S. moellendorffii. The significant hits were selected with an E value≦1e-2 as the cutoff; 517 significant hits corresponding to 461 TPSs genes (for the case of alternative splicing, the longest one was selected as the representative for that locus) were identified as putative plant TPSs from all sequenced plant genomes, and 28 known TPSs from gymnosperms, which were used in a previous analysis (Gershenzon and Dudarevam Nat Chem Biol 3:408-414, 2007), were also included. In total, 489 plant TPSs were subject to additional analysis. Identification of TPSs from Bacteria and Fungi. The annotated reference sequences for all of the bacterial and fungal proteins were downloaded from the National Center for Biotechnology Information (available on the world wide web at ncbi.nlm.nih.gov/RefSeq, RefSeqrelease44). The hmmsearch command in the HMMER package was used to search against the above protein datasets from bacteria and fungi by using the Pfam model PF03936. The significant hits were selected with an E value≦1e-2 as the cutoff; 191 TPSs were identified from bacteria, which were distributed in 88 species of seven phyla (38 species of Actinobacteria, 32 species of Proteobacteria, 7 species of Cyanobacteria, 6 species of Firmicutes, 3 species of Chloroflexi, 1 species of Bacteroidetes, and 1 species of Chlamydiae), and 56 TPSs were identified from fungi, which were distributed in 28 fungal species of two fungal phyla (23 species of Ascomycota and 5 species of Basidiomycota).

Phylogenetic Reconstruction. To understand the evolutionary relatedness of TPSs, phylogenetic trees were reconstructed using TPSs identified from S. moellendorffii, TPSs identified from the 16 plant genomes listed in Table S3, 28 known TPSs from gymnosperms, and TPSs identified from bacteria and fungi. To ensure the reliability of the phylogenetic trees, TPS fragments shorter than 200 aa were removed. TPSs were first subject to multiple sequence alignments using the MAFFT v6.603 program. Then, the aligned sequences were visualized using ClustalX2 and manually edited to remove gaps and ambiguously aligned regions to keep as many as possible informative sites for phylogenetic reconstruction. Multiple sequence alignments were systematically recalculated whenever a sequence was removed or edited in the original alignments. The ProtTest v1.4 package was used on the multiple sequence alignment to select the best-fit models for phylogenetic analyses. Maximum likelihood phylogenies were built using PhyML v3.0. Specifically, PhyML analyses were conducted with the JTT model, 1,000 replicates of bootstrap analyses, estimated proportion of invariable sites, four rate categories, estimated γ-distribution parameter, and optimized starting BIONJ tree. Phylogenetic trees were visualized by FigTree version 1.3.1 (available on the world wide web at tree.bio.ed.ac.uk). Bootstrap values are not drawn on any nodes with a value less than 50%. Identification of SmMTPSL Neighboring Genes and Their Corresponding Best Hit in NCBI. The sequences of three upstream and three downstream genes of each SmMTPSL gene are extracted using an in-house Perl script and searched against the NCBI protein nonredundant database using blastp. An E value cutoff≦1e-5 was adopted to identify significant protein matches. If there are top hits in other species for the neighboring genes of each SmMTPSL, then the NCBI accession numbers according to these top hits in other species are regarded as the candidate homolog of SmMTPSL neighboring genes (Table 2).

Gene Cloning. S. moellendorffii plants of ˜15 cm in height were subject to treatment with a fungal elicitor alamethicin. After 24 h, above-ground parts of the plants were detached and used for RNA extraction. Full-length cDNAs for selected SmTPS and SmMTPSL genes were amplified by RT-PCR and cloned into a protein expression vector pEXP5-CT/TOPO from Invitrogen. Cloning of Full-Length cDNA of Selected SmTPS and SmMTPSL Genes. S. moellendorffii plants of ˜15 cm in height were subject to treatment with a fungal elicitor alamethicin. The above-ground parts of the plants were detached and placed in a glass beaker containing 10 mL 5 μg/mL alamethicin (dissolved 1,000-fold in water from a 5 mg/mL stock solution in 100% MeOH). After 24 h, tissues were collected and used for total RNA extraction. The extracted RNA was then used for the synthesis of cDNAs, which served as a template for the amplification of full-length cDNAs of selected SmTPS and SmMTPSL genes by PCR. Amplicons were cloned into the protein expression vector pEXP5-CT/TOPO (Invitrogen) and fully sequenced.

Terpene Synthase Enzyme Assays. The catalytic activity of E. coli-expressed recombinant SmMTPSLs and SmTPSs was performed by using substrates geranyl diphosphate, farnesyl diphosphate, and geranylgeranyl diphosphate. Terpene products were identified using GC-MS. The detailed procedure for TPS enzyme assays is provided below. Liquid cultures of the bacteria harboring the expression constructs containing full-length cDNAs of selected SmMTPSLs and SmTPSs were grown at 37° C. to an OD600 of 0.6. Isopropyl β-D-1-thiogalactopyranoside was added to a final concentration of 1 mM, and the cultures were incubated for 20 h at 18° C. The cells were collected by centrifugation and disrupted by a 4°-30 s treatment with a sonicator (Bandelin UW2070) in chilled extraction buffer [50 mM Tris·HCl, pH 7.5, 5 mM DTT, 10% (vol/vol) glycerol]. The cell fragments were removed by centrifugation at 14,000×g, and the supernatant was desalted into assay buffer [10 mM Tris·HCl, pH 7.5, 1 mM DTT, 10% (vol/vol) glycerol] by passage through an Econopac 10DG column (BioRad). To determine the catalytic activity of SmMTPSLs and SmTPSs, enzyme assays containing 50 μL bacterial extract and 50 μL assay buffer with 10 μM substrate (geranyl diphosphate, farnesyl diphosphate, or geranylgeranyl diphosphate), 10 mM MgCl₂, and 0.05 mM MnCl₂ in a Teflon-sealed, screw-capped 1-mL GC glass vial were performed. A solid-phase microextraction fiber consisting of 100 μm Polydimethylsiloxane (SUPELCO) was placed in the headspace of the vial that was incubated at 30° C. for 1 h. For analysis of the adsorbed reaction products, the solid-phase microextraction fiber was inserted directly into the injector of the gas chromatograph. Assays containing geranylgeranyl diphosphate were overlayed with 100 μL hexane and extracted by vortexing. The organic phase was then removed, and 2 μL were taken for gas chromatography-mass spectrometry (GC-MS) analysis. The authentic standard for copalyl diphosphate was obtained by expressing the plasmid pGGeC, which contains the geranylgeranyl diphosphate synthase gene from Abies grandis and the copalyl diphosphate synthase gene from Arabidopsis (Cao et al. Proteins 78:2417-2432, 2010), in Escherichia coli. A Hewlett-Packard model 6890 gas chromatograph was used with the carrier gas He at 1 mL min-1, splitless injection (injector temperature=220° C., injection volume=1 μL), a Chrompack CP-SIL-5 CB-MS column [(5%-phenyl)-methylpolysiloxane, 25 m°-0.25 mm i.d.°-0.25 microfilm thickness; Varian], and a temperature program from 50° C. (3 min hold) at 6° C. min−1 to 240° C. (1 min hold). The coupled mass spectrometer was a Hewlett-Packard model 5973 with a quadrupole mass selective detector (transfer line temperature=230° C., source temperature=230° C., quadrupole temperature=150° C., ionization potential=70 eV, and scan range=40-350 atomic mass units).

Headspace Analysis. Headspace collection and volatile identification were performed as described below. Above-ground parts of control and alamethicin-treated S. moellendorffii plants were detached and placed in a glass beaker. Volatiles emitted from S. moellendorffii samples were continuously collected by pumping air from the chamber through a SuperQ volatile collection trap. Collected volatiles were analyzed using GC-MS. Above-ground parts of S. moellendorffii plants were detached and placed in a glass beaker containing either 100 mL 5 μg/mL alamethicin in 0.5% EtOH or 100 mL 0.5% EtOH as a control, and the glass beakers were placed in a glass chamber for headspace collection using an open headspace sampling system (Analytical Research System). Volatiles were continuously collected by pumping air from the chamber through a SuperQ volatile collection trap. After 24 h, the volatiles were eluted from the SuperQ trap using 100 μL methylene chloride containing 1-octanal [0.003% (wt/vol)] as an internal standard. Volatiles were analyzed using GC-MS as described for enzyme assays.

Gene Expression Analysis Using Real-Time RT-PCR. Above-ground parts of S. moellendorffii plants were detached and placed in a glass beaker containing either 100 mL 5 μg/mL alamethicin in 0.5% EtOH or 100 mL 0.5% EtOH as a control. After 6 h, tissues were collected and subject to RNA extraction. Real-time RT-PCR experiments were conducted as previously described (Christianson, Curr Opin Chem Biol 12:141-150, 2008). Expression values of each gene were normalized to the expression levels of the 6-phosphogluconate dehydrogenase (Sm6PGD) in respective samples (Facchini and Chappell Proc Natl Acad Sci USA 89:11088-11092, 1992). The primers for target genes were designed using Primer Express software (Applied Biosystems), whereas the primer sequences for Sm6PGD were designed as previously described (Facchini and Chappell). The primers used were: 5′-GGCTATTCTATCCATTGTGAG-3′, SEQ ID NO. 90 (forward) and 5′-TCACACCACACATTGATCTC-3′ SEQ ID NO. 91 (reverse) for SmMTPSL1,5′-GAAAGTTCTTCGCTTCGTTC-3′ SEQ ID NO. 92 (forward) and 5′-TGTATGCAGTTGCCACATTC-3′ SEQ ID NO. 93 (reverse) for SmMTPSL17, 5′-TTTCCAGGACGTAGTTGACC-3′ SEQ ID NO. 94 (forward) and 5′-CAAACTTAGTCAGCTCTGAG-3′ SEQ ID NO. 95 (reverse) for SmMTPSL22, and 5′-CGTTCTTGTCAATGATCTCC-3′ SEQ ID NO. 96 (forward) and 5′-CATACCTTGTCCACAGTCTC-3′ SEQ ID NO. 97 (reverse) for SmMTPSL26.

### Example 6

### Generation of Stable Transgenic Tobacco

Constructs, which include the disclosed S. moellendorffii terpene synthases, are tested for activity tobacco transformation with a nucleic acid construct containing a terpeene synthase nucleic acid under the control of a promoter, such as a constitutive or inducible promoter are transformed into tobacco. Tissue-specific expression of each construct is analyzed in the stable transgenic tobacco. In some examples, production of terpenes is analyzed, for example in the head space.

### Example 7

### Generation of Stable Transgenic Arabidopsis

Constructs, which include the disclosed S. moellendorffii terpeene synthases, are tested for activity in a model dicot (Arabidopsis thaliana) after transformation with a nucleic acid construct containing a terpeene synthase nucleic acid under the control of a promoter, such as a constitutive or inducible promoter are transformed into are transformed into Arabidopsis thtaliana, such as Arabidopsis thaliana, Columbia seeds. Arabidopsis thaliana plants are then transformed using the methods available to those of ordinary skill in the art, for example transformed using an Agrobacterium system by electroporation, as described in Clough and Bent, Plant Journal 16:735-743, 1998, which is specifically incorporated herein by reference. In some examples, T1 seed is harvested form the plants and germinated. Transformed seedlings are identified, for example using molecular biology techniques, such as PCR to identify the transgenics. In some examples, the construct includes a gene of interest operably linked to the promoter and activity and/or expression is measured. Tissue-specific expression of each construct is analyzed in the stable transgenic Arabidopsis plants.

### Sequences

SEQ ID NOS: 1-47 are exemplary nucleic acid sequences of Selaginella moellendorffii terpene synthases from the SmMTPSL genes.

(SEQ ID NO: 1)
ATggctattctatccattgtgagCATTTTTGCAGCGGAGAAAAGCTACTC
CATTCCACCAGCAAGTAATAAACTTCTGGCCTCTCCAGCGCTGAATCCGC
TGTATGATGCAAAGGCCGACGCTgagatcaatgtgtggtgtgaCGAGTTT
CTGAAGTTGCAACCTGGAAGCGAGAAATCTGTGTTTATTCGAGAGAGCAG
GCTTGGATTGCTCGCAGCTTATGCATACCCGAGCATTTCATACGAGAAGA
TTGTTCCCGTTGCAAAGTTCATCGCTTGGCTCTTTCTTGCAGATGACATT
CTGGATAACCCTGAGATCTCTTCGTCGGACATGAGGAACGTGGCAACCGC
ATACAAGATGGTTTTCAAGGGAAGATTTGACGAGGCCGCACTTCTGGTCA
AGAATCAGGAGCTGCTGAGGCAAGTGAAGATGTTATCTGAGGTTTTGAAA
GAACTGTCCCTCCATCTAGTGGACAAATCCGGCCGATTCATGAATTCTAT
GACCAAGGTGCTCGACATGTTTGAGATTGAATCGAACTGGCTTCACAAGC
AAATCGTTCCCAACCTGGACACGTACATGTGGCTGAGAGAGATCACATCT
GGTGTTGCGCCTTGCTTTGCTATGCTTGATGGTTTACTGCAACTTGGGCT
GGAAGAGCGTGGCGTGCTGGATCATCCTCTCATACGCAAGGTTGAGGAGA
TTGGGACGCACCACATTGCGCTCCACAATGACTTGATCTCGTTCAGGAAG
GAGTGGGCGAAAGGGAACTACCTCAACGCCGTGCCCATTCTCGCCAGCAT
TCACAAGTGTGGTTTGAACGAGGCGATTGCCATGTTGGCGAGCATGGTGG
AGGATTTGGAGAAGGAGTTCATCGGGACAAAGCAGGAGATCATTTCAAGT
GGGCTTGCCAGGAAGCAAGGCGTCATGGATTATGTGAATGGGGTAGAGGT
GTGGATGGCCACAAACGCAGAATGGGGATGGTTGAGTGCTAGATACCATG
GAATTGGGTGGATCCCTCCTCCAGAAAAATCAGGGACCTTCCAACTCTAG
(SEQ ID NO: 2)
ATGGCTTTAGCTCTAGACAAGATCTATGCTATTGAGAAGTTGCTAGGCCT
CAAGAATTTCCACCTCCCAAAGATCCCTTGCTCCATTCCTTCAGTCCCTT
GCCATCCAGATAGCATCTATGCATCCAACAAGGCCCATGAATGGGCATAC
AAGTTCATGGATCCAAAAATGACAGCCGCTGATAGAAAGGCTTTGGAAGA
TTGGAAAATCCCAATGTTTGCAACCCTCGTAGTGCCATTTGGATCCAAGA
GAAATGCTGTCATTTGCTCAAAGTATAGCATGTTTGCCTTATTAGTGGAC
GACTCGGTTGATGAGGGCTTCGTTGAAAGTACCATTCTTCAAGATTACTA
TTCCACAATCCTTAATCACCTCCATAATCCTAATTTCAAGATCCAGGCAT
CGGACGATCACCTTCCACTTCGAGTTTACAGGGCCACTGAAGAGCTTGTT
ACTGAGATAAGATCATCCATGCTTCCTCCAGTGTATGCTCATTTTGTAGC
ACAGTTTGAGAGGTATGCACTCAGCAGAATGGCAAGCAGGCCCAAGTTTC
AATCTGTCAAGCAGTATATCGAATGGAGAAGGTTTGATGTGTTCTTAGAG
CCTATCTTCAGCTTCATAGAGATGGCACTTGAAGTCGCAGTTCCGGACAC
GGAACTGGAATCAGAGGACTATCTAATTCTGCGAGATGCTGGAATTGACT
ATATATCTATGTACAATGATGTTCTCTCGTTTGCAAAGGAGTTTGCATGC
AACAAACTGCTGAACCTTCCAGTGTTGCTGCTTCTTTCGGATCCGGAGGT
GGAGTCATTCCAGAATGCAGTGGACAAGAGTTGCAAGATGATCGTGGACA
AGGAGCAAGAATTTGTATACTACCACAACATTCTGATCACTCAGGCAAGA
GGTGAAGGTAAACACGCGTTTGTGAAGTATCTTGAGTGTCTTCCTACTGT
TCTTTCCAACACGCTTTACTATCACTACTCCTCTGCCCGTTACCATCCAG
CTTTCATAACGGGTGAGAAGTTTGATGCGAATTGGTGCTTGGACACTGTT
ATAAACCATAGAAGAACTGGCCGGTGA
(SEQ ID NO: 3)
ATGGCCGCACCTTCTATCTATCGTCCCCAAATTCTGGAGCAGCTCCTCGC
CTGCAAGAGCATCTACTTGCCTCAAATTCGCTGCTCGTTGCCATTGCAGT
GCCACCCAGACTACGCCTCCGTCTCCAAACAGGCGAACGATTGGGCCTTC
CGCTTCCTCAAGATCAATGCCACCAATGCCGCTGCCGATAAGAAATACTT
CACCCAGTGGAGGATGCCACTCTACGGCACCTTTGTTGTGCCTTGGGGCG
ACTCCAGGCACGCTCTAGCGGCCGCCAAGTACACCTGGCTTATCACCATT
CTCGACGATGCGGTCGACGAGGAGCCTTCGCAGCGGGACGAGATCCTGGA
AGCTTACATGAGCCTTGCCTCCGGTCAAAGATCCATCGCCCAAGTTCCCA
ACAAGCCCGTGCTCGTCGCCCAAGCCGAGCTCATCCCGGATCTGCAGAAG
CTCATGTCGCCGCTCCTCTTCCAGCGGCTGCTCGTCTCGTACAGGAAATT
TGTTGGCTGCTACTCGGCCAAAGTCGACGAGGAGGAGTTCACGAAAGAGT
CTTACGCTGTGCATCGCCGGGAGGACTACGTTGTCAAGCCGATGCTTAAC
TTCACGCAGATGTGCCTGGGAGTCGAGCTGAGAGACAAGGATCTGGAAAG
CGAGGAGTACCTGCGGGCGATAGATGCCATGTTTGATCATATGTGGCTGG
TGAACGACATCTTTTCATTCCCAAAGGAGCTGAGGAAGAAAACTTTCAAG
AACATAATTTTTCTCTTGCTCTTCACGGACCACACCGTTCGCTCTGTTCA
ACAGGCAGTCGATAAGGCGAACGCCATGATTCAGGAAAAAGAACAAGAAT
TCATGTATTACCACGAGATCCTGACGAGGAAAGCAATGGAATCTGGCAAC
CACGACTTTCTGGCGTACCTTAGAGCGATTCCGGCATTCATCCCTGGAAA
TCTACGTTGGCACTACCTCACAGCTCGGTACCACGGTGTTGATAATCCAT
TTGTAACAGGAGAGCCATTCAGTGGGACTTGGTTGTTTCATGATACGCAG
ACTATCATACTCCCCGAGTACAAACCAACTCATCCCCATCTGCAAGTCTG
A
(SEQ ID NO: 4)
ATGGCCGCGCCTTCTATCTATCGTCCCCAAATTCTGGAGCAGCTCCTCGC
CTGCAAGAGCATCTACTTGCCTCAAATTCGCTGCTCGTTGCCATTGCAGT
GCCACCCAGACTACGCCTCCGTCTCCAAACAGGCGAACGATTGGGCCTTC
CGCTTCCTCAAGATCAATGCCACCAATGCCGCTGCCGATAAGAAATACTT
CACCCAGTGGAGGATGCCACTCTACGGCACCTTTGTTGTGCCTTGGGGCG
ACTCCAGGCACGCTCTAGCGGCCGCCAAGTACACCTGGCTTATCACCATT
CTCGACGATGCGGTCGACGAGGAGCCTTCGCAGCGGGACGAGATCCTGGA
AGCTTACATGAGCCTTGCCTCCGGTCAAAGATCCATCGCCCAAGTTCCCA
ACAAGCCCGTGCTCGTCGCCCAAGCCGAGCTCATCCCGGATCTGCAGAAG
CTCATGTCGCCGCTCCTCTTCCAGCGGCTGCTCGTCTCGTACAGGAAATT
TGTTGGCTGCTACTCGGCCAAAGTCGACGAGGAGGAGTTCACGAAAGAGT
CTTACGCTGTGCATCGCCGGGAGGACTACGTTGTCAAGCCGATGCTTAAC
TTCACGCAGATGTGCCTGGGAGTCGAGCTGAGAGACAAGGATCTGGAAAG
CGAGGAGTACCTGCGGGCGATAGATGCCATGTTTGATCATATGTGGCTGG
TGAACGACATCTTTTCATTCCCAAAGGAGCTGAGGAAGAAAACTTTCAAG
AACATAATTTTTCTCTTGCTCTTCACGGACCACACCGTTCGCTCTGTTCA
ACAGGCAGTCGATAAGGCGAACGCCATGATTCAGGAAAAAGAACAAGAAT
TCATGTATTACCATGAGATCCTGACGAGGAAAGCGATGGAATCTGGCAAC
CACGACTTTCTGGCGTACCTTAGAGCGATTCCGGCATTCATCCCTGGAAA
TCTACGTTGGCACTACCTCGCAGCTCGGTACCACGGTGTTGATAATCCAT
TTGTAACAGGAGAGCCATCCAGTGGGACTTGGTTGTTTCATGATACGCAG
ACTATCATACTCCCCGAGTACAAACCAACTCATCCCCATCTGCAAGTCTG
A
(SEQ ID NO: 5)
ATGGCTCCCTACGATTTCGTTCCAAATGTGCAGTGTTCGTTCCCTGTGAA
GTGCCACCCTCTGTATTCTTTCATTCGTCCAGGCTTGGAAGATTGGGCTG
CAACTTTGGAGCCTGGGCATGGTGAAGGGAACCCGAAAGGCCTGGGAGCT
GACTTGGGAGGTGCCAAGAGGCTTGTTGATAGCTACCTTGGCATAATCCA
TGCCCCGGAACCCGTGGCAGATATGGAATTTCCACGGTTCTGTGATATGT
GGAATGATCTACGTGCAGATATGCCACTCAAGCAGTACCAGCGATTTGCC
AACAGAGTGTCCGAGCTGTTGAAGGCAAGTGTGAATCAGGTGAGGCTAAG
GAATCTGAAAACGGTGATGGGCTTGGAGGAGCTGCTGGCTCACCGTCGCA
TGTTAGTTGGTGTATTTGTTATGGAAACTCTAATGGAGTATGGCATGGGA
TTCGAACTCCAGGACGACGCCATTTCAAATCAGGACCTCCAAGAGGCTGA
AAGTCTGGTTGCAGACCACTGCAACTGGCGATACTCAGTTCAATATCGTC
CATGCGGCAATTCGGATTCAAAGGGCTTTTCTTTCGAGTATGCAGCCGAC
AAAGTTCAAAAACTGGTGCAGAGTATCGAGCATCGATTCAAGAAGCTGTG
CGAGAATATCAGAAGATCAAGCTGCTACAATGGTGCAATGGAGGCTTACC
TGGAAGGCTTGTCTCATATTATATCCGGAAACCTTGAGTGGCACCGGCAG
ACAGGACGATACAAACTGGTATCTTGA
(SEQ ID NO: 6)
ATGGCTGCCAGCGTCAATGGCGTGCTCCCGGAGCTCTCCACTCTCTCAAA
ATTTGAACTCCGTCCATTGCCCTGCGCGTTTCCTTTCGAGTGCCACCCAA
ATCACGCGTCGCTCACCCGAGAGGTTGACGAGTGGGCGATCCGATCGCTG
CAAGCCCGGGGCTCCATGCCCAAGCGCCAGATGATCATCGAGTCCAAGAT
CTCGGCGGCGGCATGCATGACTATCCCGCGTGGCCGGGACGATCGTAAGA
TGGTGCTGGCGGGCAAGCATTTGTGGGCGCTCTTCTTGCTGGACGACGCG
CTGGAATCGTGCCGGAGCCAGGAGGCCGCGAGAGTCCTCGCCCGGCGAGC
GATGGAAGTCGCGAGAGGGGACCAATTGGAAGGGATGATCCAGGAGGAAA
GAGAACTAGAAGAAGCCAAAGGGGTCGCGAGGAAATTCGCGATCCAGGAA
GAAGAAGGAGATCGATATAATGATCAGTCGAGAGGAATCCTTGCAAACAT
AGCGATCCAAGAGGACCCTGGTCTCATCGATCTGGCTACCAGAGGAATGG
CGACGAAAATCGCAATCACGGAAGAAGATCAAGGTCGCGATTCTCGATGG
GCGCTGGGATTGTTCCGGGAAGTAGTGGCGGAGCTCCGGCGATCAATGCC
GCTCCCGATGTTCGATCGCTACCTGCGGTACCTGGATCGCTACCTGGAGG
CCGTGATCCAGGAGGTGGGATACCAGATCGCGGGCCACATCCCGCGGGAG
GACGAGTATCGCGAGCTCCGGCGGGGAACGTCCTTCACAGAGGGCACCAG
CGCGATCTTTGGCGAGCTGTGCATGGGGCTGGAGCTCCACGAATCTGTGA
CATCGTCTCGCGATTTCATCGAATTCGTGGCGCTCGTCGCGGACCACATC
GCGCTCACCAACGATGTCCTCTCCTTCCGCAAGGATTTCTACGCCGGGGT
CGCCCACAACTGGCTCGTCGTGCTCCTCCGCCACAGCCACCGCGGGACCG
GCTTCCAATCCGCGCTGGACAGCGTCTATGGCATGATCCGCGACAGCGAG
TGCCGGATCCTGGGGCTCCAGTCGCGAATCGAGGCGCAAGCACTGAAGAG
TGGCGATGGTCACCTCCTCAGCTTCGCGCAGGCGTTTCCCCTGTGCCTGG
CCGGGAATCGGAGGTGGTCATCGATCACCGCGCGATACCATGGCATTGGG
AATCCTCTCATCACTGGCGTGGAGTTCCACGGGACATGGCTCTTACATCC
GGATGTCACCATAGTTATTTGA
(SEQ ID NO: 7)
ATGGCACTTGCCGTGGAGAAGATTCCCGCCATGGAACACCTCCTGGGGCT
AAAGAGGTTCTATTTACGGCCCATTCGCTGCTCCATCCCCTCCAGCGCCT
GGCATCCCGACCACAAGCTCGTTGCCAAGCTCGCGAACGAGTGGGCATTC
CCATTCATCAATCCCAGCATGAGCGATGCCCAAAAGCTCTCCCTGGAGCG
CATGCGAATCCCGCTCTACATGAGCATGCTCGTGCCGTGCGGATCCACCG
AGTTCGCGTGGTTTGGAACGCGATCATGCTGGATGATCTCCTCGAGGACG
AGTCCCCCAGCGGCGCCCCCCGGGAGGAGTTCCTGGAGACTTTCCAGGGC
ATCCTCCACGGGGCGCACCCACATCGCGATCCAGTCCATCCATCGCTCGA
GTTCTGCGCGGACCTCATTCCGCGCCTGCGATCATCCATGGCTCCCCGGG
TGTGGTCGCGCAGATGGAGGCCCTACGCTGCCTCCATGGACCGGAGCGTC
CTTTCTCTAGCACAATCGGCGTTGACGGTCGAGCCCGCAGGAGGCTCGAT
TGCTTCCTCCTCCCCTGCTTCCCATTCATCGAGATGTCGCTGGAGATTGC
GCTCCCAGACAGCGATTTGGAGTCGCGGGACTACCTGGCGCTCCAGAATG
CCATCAACGACCACGTCCTCCTTGTCAACGACGTTATCTCCTTTCCCGCG
GAGCTGCGCGCCAAAAAGCCACTGAGAAGCATCGCGTCCTTGCAGTTGCT
CTTGGATTCCCAGCATCAACACGCTCCAGGAATCGGTGGACAGAACCTGT
GCGATGATCCAGGAGAAGGAACGCGAGGTGACGCATTACTACGACGTTGT
GATGAGAAACGCTGTGGCTTCTGGCAATGCCGAGCTTGTGAGCTACCTTG
AGATCCTCAAGCTGTGCGTTCCAAACAACCTCAAGGTCCACTTCATTAGT
TCTCGTTATGGAGTGAATGATGGCGAGTCTGGTCATGGAATTTGGATTGT
TCTGTAG
(SEQ ID NO: 8)
ATGGCACTTGCTGTGGAGAAGATTCCCGCCATGGAACACCTCCTGGGGCT
AAAGAGGTTCTATTTACGGCCCATTCGCTGCTCCATCCCCTCCAGCGCCT
GGCATCCCGACCACAAGCTCGTTGCCAAGCTCGCGAACGAGTGGGCATTC
CCATTCATCAATCCCAGCATGAGCGATGCCCAAAAGCTCTCCCTGGAGCG
CATGCGAATCCCGCTCTACATGAGCATGCTCGTGCCGTGCGGATCCACCG
AGAGCGCCGTCCTCTGCGGCAAGTTCGCGTGGTTTGGAACCATGCTGGAT
GATCTCCTCGAGGACGAGTCCCCCGGCGGCGCCCCCCGGGAGGAGTTCCT
GGAGACTTTCCAGGGCATCCTCCATGGGACACACCCACATCGCGATCCAG
TCCATCCATCGCTCGAGTTCTGCGCGGACCTCATTCCGCGCCTGCGATCA
TCCATGGCTCCCCGGGTGTACGCGCACTGGGTCGCGCAGATGGAGGCCTA
CGCTGCCTCCATGGACCGGAGCGTCCTTTCTCTAGCACAATCGGCGTCGA
CGGTCGAGAGCTACTTGGCGCGCAGGAGGCTCGATTGCTTCCTCCTCCCC
TGCTTCCCATTCATCGAGATGTCGCTGGAGATTGCGCTCCCAGACAGCGA
TTTGGAGTCGCGGGACTACCTGGCGCTCCAGAACGCCATCAACGATCACG
TCCTCCTTGTCAACGACGTTATCTCCTTCCCCGCGGAGCTGCGCGCCAAA
AAGCCACTGAGAAGCATCGCGTCCTTGCAGTTGCTCTTGGATCCCAGCGT
CAACACGTTCCAGGACTCGGTGGACAGGACCTGTGCAATGATCCAGGAGA
AGGAACGCGAGGTGACGCATTACTACGACGTTGTGATGAGGAACGCTGTG
GCTTCTGGCAATGCCGAGCTTGTGAGCTACCTTCAGATCCTCAAGATGTG
CGTTCCAAACAACCTCAAGGTCCACTTCATTAGTTCTCGTTATGGAGTGA
ATGATGGCGAGTCTGGTCATGGAATTTGGATTGTTCTGTAG
(SEQ ID NO: 9)
ATGTCGAGCTTTCGGAGTCTCATGGATCCCCCACTGTTTGCTCGCTATAT
GATTTGTTTGAAAACATTTCTGGATTCTTTGGTGGAGGAGGCCTCTTTGC
GATCTGCCAAATCCATCCCAAGTCTCGAGAAATATCAGTTGCTCCGGAGA
GGGACAGTTTTCGTTGAAGGAGCCGGAGATTTTGTAGCATTTGTCAACGC
AGTGGCTGATCATGTTCTCTTCTCCTTCCGACACGAGATGAAAATCAAGT
GCTTCCACAACTATCTCTGTGTCATCTTTTGCCACAGCCCGAATAATGCA
AGCTTTCAAGAGGCTGTCGACAAAGTATGCAAAATGATCCAGGAGACCGA
AGCCAAGATCCTTCAACTCCAAAAGAAGCTGATGAAGATGGGCGAGGAAA
CTGGGAACAAAGACCTGGTGGACTATGCAACATGGTATCCTTGTTTTACA
TCTGGACATCTCCGCTGGCCATGGGCTTGA
(SEQ ID NO: 10)
ATGTCGAGCTTTCGGAGTCTCATGGATCCCCCACTGTTTGCTCGCTATAT
GATTTGTTTGAAAACATTTCTGGATTCTTTGGTGGAGGAGGCCTCTTTGC
GATCTGCCAAATCCATCCCAAGTCTCGAGAAATATCAGTTGCTCCGGAGA
GGGACAGTTTTCGTTGAAGGAGCCGGAGGCATTATGTGTGAGTTTTGCAT
GGATCTCAAGCTGGATAAGGTGGCTGATCATGTTCTCTTCTCCTTCCGAC
ACGAGATGAAAATCAAGTGCTTCCACAACTATCTCTGTGTCATCTTTTGC
CACAGCCCGAATAATGCAAGCTTTCAAGAGGCTGTCGACAAAGTATGCAA
AATGATCCAGGAGACCGAAGCCAAGATCCTTCAACTCCAAAAGAAGCTGA
TGAAGATGGGCGAGGAAACTGGGAACAAAGACCTGGTGGACTATGCAACA
TGGTATCCTTGTTTTACATCTGGACATCTCCGCTGGGTGTATGTCACAGG
ACGCTACCATGGGCTTGACAATCCGCTGCTGAACGGTGAACCATTCCATG
GGACTTGGTTTCTACATCCAGAAGCCACCTTCATTCTACCATTCGGATCC
AAATGTGGATTTATTAACACCATGTGA
(SEQ ID NO: 11)
ATGGCATTGCCCAGCCTGCTCTCAACAAAGCTCAAGCCGCTTGAGCTCCT
GTCCGGTGTCACTCATTATGATCTTCCGCCAATTCCCTGTTCTCTTCCTG
TCAAGTGTCATCCTCAATTTGCTAAGTTTTCTCGCATTGCCGATACATGG
GCCATCGACGCAATGCAGCTGCAAAATGATCCATGTGGAAAGCTCAAGGC
TGTGCAGAGCCGAGCCCCGCTGCTTTACTGCTTCCTCGTCCCTTTCGGCA
TCGGAGAGGAAGAAATGATTGCAGGCTGCAAGTACAGCTGGTCGACTTCC
TTCGTGGATGATCCATTTGACGAAGAAACGGATTTGAAGCGGGCCAAGGA
ATGGAAGAAGGTCGTGCTGCGAGCTGCGAACGGTACTCCCAGTGCTGAAG
ATTTGATGATAAGGACGATAAAAGCTTATTCGGAGATTATGATGCACCTG
CAACAGATGATGGCGGCCCCAGTGTTTTCGAGGTTCATGAGGGCTCACTA
CGCTTGGGCAGATCACTGCGTGGAGCTTGTTCGTAGAAGGCAGCATAAAG
ACCCTCCAACTGTAGCCACATACCTTGCAGACAGGTGCGAAAATCTGCTC
GTAGAACCGATCTTCATTCTGGCGGAGGTGTGCATGAAGCTTCAGATTGA
CCCGGAGTTCCTGTCGCTGCCAGAGTTCAAGAAAATCTGGACCACAATGC
TGGAACATGCGGCTATCGTGAACGACGTCTTGTCAATCCGTGTAGACATC
CTCAACGGACACTACTACACCTATCCTGGCCTCGTCTTCCAGCAGCATCC
TGAGATCCAAACTTTCCAGGAGGCTGTGGACTATTCCGTGGGGATGATCC
AGACCAAGGAGAGAAAGTTCATCAAACTGCACGAGATGCTGACCGACAAA
GCCAGGCAATGCGGCTTCAAGAACAAGTCCGACTTGCTCAAGTATGTTGA
AGCTTTGCCAAACTTCATCGCTGGAAATCTTTACTGGCACTACCTTAGCG
CCAGATACTTCGGTGTCAACAACCCCTTCCTCACCGGAGAGCCTGTCCAA
GGCACCATCCTCATCCATCCCCGCAACACAGTCGTGCTCCCACCTTACCA
GCGAAACAAGCACCCCTTTCTCATCGATGTCGACAATCTGGAGCTCGGTG
CGTGA
(SEQ ID NO: 12)
ATGAGGAGCTTCAGCAGCTTCCACATCTCCCCAATGAAATGCAAGCCTGC
ATTGCGAGTCCATCCATTGTGTGACAAGCTCCAGATGGAAATGGACCGCT
GGTGTGTAGACTTCGCTTCGCCAGAGTCCTCGGACGAGGAGATGAGGTCC
TTTATAGCTCAGAAGCTGCCCTTTCTCTCGTGCATGCTCTTCCCCACAGC
GCTCAACTCAAGGATCCCATGGCTGATCAAGTTCGTATGCTGGTTCACAC
TGTTCGATTCGCTCGTCGACGACGTCAAGTCCCTGGGCGCGAATGCCCGA
GACGCGTCGGCGTTCGTGGGTAAGTACCTTGAAACCATCCATGGAGCTAA
AGGGGCGATGGCGCCGGTGGGAGGCTCGCTCCTCTCGTGCTTCGCCTCGC
TGTGGCAACACTTCCGCGAGGACATGCCGCCGCGGCAGTACTCGCGCCTG
GTGCGCCACGTGTTGGGCCTGTTCCAGCAGTCGGCTTCGCAGTCCCGGCT
CCGCCAAGAGGGCGCCGTCCTCACGGCCAGCGAGTTTGTGGCCGGGAAGC
GCATGTTTAGCTCGGGGGCGACGCTGGTTTTGCTCATGGAGTATGGACTG
GGGGTGGAGCTCGACGAGGAAGTGCTCGAGCAGCCGGCCATCCGGGACAT
TGCGACGACCGCCATCGACCACCTCATCTGCGTCAACGACATCCTCCCCT
TCCGTGTGGAGTATCTCTCCGGGGACTTCTCCAACCTCCTCTCCTCGATT
TGCATGTCCCAGGGCGTCGGATTGCAAGAGGCGGCGGACCAAACTCTCGA
GCTGATGGAGGACTGCAACCGGAGGTTCGTGGAGCTGCACGACTTGATAA
CCAGGTCGAGCTACTTCTCCACCGCTGTGGAGGGCTACATTGACGGCCTT
GGCTACATGATGTCCGGGAACCTTGAGTGGAGCTGGTTGACTGCCCGCTA
CCATGGTGTGGACTGGGTAGCGCCAAACTTGAAAATGCGGCAGGGGGTGA
TGTACCTTGAAGAACCACCACGTTTTGAGCCAACTATGCCACTAGAAGCT
TACATTTCGTCTAGTGATTCTTGCTAG
(SEQ ID NO: 13)
ATGGAGGCTATTGTTTCATCCAGCAAGATCCATGCAGTAGAACATTTGCT
GAGCCTCAAGAGCTACTCTCTCCCTCAAATCCTCCTTGCCCATCCCGTCA
AGTGTCACCCCGACTACACCTCGATCTGCAAGGAATCGGACGAATGGATC
TTCAGCTACCTCGGCGTCACAAGCCCGGAACACAAGAAGCGCTTAGCGCA
ATGGAGGGTCCCAATCTTCGCCGCCTTCCTGACGCCCCCCAGCAGCCCCA
AGAGGCGCACGCTTTTGGGCGGCAAATTTACGTGGCTGATCACTGCGCTG
GATGATCAGCTGGACGAGAGCAAGATCTCCCAAGCTGGGCGGAGCTGCCA
GTACAGGGACGCCATCTTGAGCATTTTCTCCGGCAGAAGCGATTACCCGG
CCATACTCCCGGCGGAGGTTCCTCTTCTCAGAGCCTGCGAGGAGCTCATG
CCGGAAATCCGCTCCTTCATGCTTCCGCCCACTCTCAATCGCTTCCTTGC
TTACACCAAGCAGTGGTCGCAGACTTTCGATGTTGCCTATGAGAGCACAC
AAGTGTTCAAGGAGCTAAGGAGGGACAACGTTTGGATCACCGCATACTTC
CCGATGATCGAGATGTTCCTGGGATTGGGTCTTGGGGACGATGTGGCCGG
GTCCAAGGATTTCCTCGCCGCTCAGGACGCAATATCGGACCATGCCTGGA
TGGTGAACGACCTCTTCTCTTTCGCCAAAGAGTTCCGGGACGAGAAAAAG
CTCAGTAACATTCTGTCCGTGAGCTTGCTCATGGATTCGTGCGTGCACAC
CATCCAGGACGCCATTGATCTTCTGTGTACCGAGTTGCAAGCAAAGGAGG
AGGAGTTTCTCTACTACCACGGGATCCTTGTCAAGCGAGCCCAAGCAGGG
AACAACCAAGATCTCTTAAGGTACCTAGAGGCAATCCTTGCCGTGATCCC
AGGTAATTTACACTTTCACTACATAACGGCGCGTTACCACGGATACAATA
ATCCATGTGTAAATGGAGAAGCATGGCATGGTAAAGTTATATTGCAACCA
AATACTCTCGGGCCACCACCAAAGCCACATCCATACCTCTATGACATATA
A
(SEQ ID NO: 14)
ATGGCTGTTTCATCCATTGTGAGCATTTTCGCAGCAGAGAAAAGATATTC
CATTCCACCAGTGTGTAAACTCCTTGCCTCTCCAGTGCTGAATCCTCTGT
ACGATGCAAAGGCCGAGTCTCAGATCAATGCGTGGTGCGCCGAGTTTCTG
AAGTTGCAACCTGGAAGCGAGAAAGCTGTGTTTGTTCAAGAAAGCAGGCT
TGGATTGCTCGCGGCTTATGTTTACCCGACCATTCCATACGAGAAGATTG
TTCCGGTTGGAAAGTTCTTCGCTTGGTACTTTCTTGCAGATGACATTCTG
GATAGCCCGGAGATCTCCTCGTCGGACATGAGGAATGTGACAACTGCATA
CAAGATGGTTTTAAAGGGAAAATTCGACGAGGCCACGCTTCCAGTGAAAA
ATCCGGAGCTGCTGAGGCAAATGAAGATGTTATCTGAGGTCTTGGAAGAA
TTGTTCCTCCATATAGTGGATGAATCAGGCCGATTCGTGGATGCTCTGAC
CAAGGTGCTCGACATGTTTGAGATTGAATCGAGCTGGCTTCGCAAGCAAA
TCATTCCCAACTTGGATACGTACCTCTGGCTGAGAGAGATCACATCTGGT
GTTGCTCCTTGCTTTGCTCTGATTGATGGTTTACTGCAACTTAGGCTGGA
GGAGCGTGGCGTGCTGGATCATCCTCTCATACGCAAGGTTGAGGAGATTG
GGACGCACCACATTGCGCTCCACAATGACATGATCTCGTTCAGGAAGGAG
TGGGCGAAAGGATACTACCTCAATGCCGTGCCCATTCTCGCCAGCAATTG
TAAGTGTGGCTTGAACGAGGCAATTGGCAAGGTTGCGAGCATGGTGGAGG
ATGTGGAGAAGGATTTCGCCCAGACAAAGCATGAGATCGTTTCAAGTGGG
CTTGCCATGAAGCAAGGAGTCATGGACTATGTGAACGGGATAGAGGTGTG
GATGGCCGGAAACGTAGAATGGGCATGGACGAGCGCTAGATACCATGGAA
TTGGGTGGATCCCTCCTCCAGAAAAATCAGCGACCTTCCAACTCTAG
(SEQ ID NO: 15)
ATGGCTCGCACGTTATTCAACGACATGCTCAAGCAGGCAGCCCTCCCTGA
CATCGTCACTTTCTCGACACTAGTGGAAGGATACTGTAATGCTGGACTGG
TGGATGACGCCGAGAGGCTTCTGGAAGAGATAATTGCCAGTGACTGCTCT
CCGGACGTGTATACGTACACAAGCCTGGTCGACAGCTTCTGCAAAGTCAA
AAGAATGGTGGAGGCGCACAGAGTTCTCAAGCGAATGGCCAAGCGGGGAT
GCCAACCCAACGTGGTGACTTACACTGCTCTCATTGACGCGTTCTGCAGA
GCTGGGAAGCCGACGGTGGCTTACAAGCTGCTGGAGGAGATGGTTGGCAT
TAACAACGACGTCCAGCCGAACGTTCAGGAGCTGGCTTCTGTGGGACTGG
GGACCTGGAAGAGGCTCGCAAGATGCTCGAGAGACTGGAGCGCGACGAGA
ACTGCAAGGCGGATATGTTCGCATACAGGGGGGCTGTGCCAGGGGAAAGA
GCTCAGCAAAGCCATGGAAGTTTTGGAAGAGATGACGCTCTCAAGGAAAG
GCAGGCCAAATGCCGAGGCTTACGAGGCGGTGATCCAGGAGCTAGCGAGA
GAAGGGAGGCATGAAGAGGCAAACGCGCTCGCAGACGAATTACTGGGTAA
CAAAGGCCACCTTCTTTCAGTGTTTAAAATTCACTTAGGAAGCATCCATT
GCGAGCATTTTCGCAGTGGAGAAAAGCTATTCCATTCCACCAAAAGCAGG
CTTGGATTGCTCGCGGCTTATGTTTACCCGACCATTCCATACGAGAAGAT
TGTTCCGGTTGCAAAGTTCATCGCTTGGTTCTTTCTTGCAGATGACATTC
TGGATAGCCCGGAGATCTCCTCGTCAGACATAAGATATGTGGCAACCGCA
TACAAGATGGTTTTCAAGGGAAGATTTGACGAGGCCACACTTCCAGTGAA
AAATCCGGAGTTGCTGAGGCAAATGAAGATGTTAGCTGAGGTTTTGGAAG
AACTGTCCCTCCATATAGTGGATGAATCAGGCCGATTCGTGGATGCTATG
ACCAAGGTGCTCGACATGTTTGAGATTGAATCGAGCTGGCTTCGCAAGCA
AATCATTCCCAACCTGGATACGTACCTCTGGCTGAGAGAGATCACATCTG
GTGTGGCTCCTTGCTTTGCTCTGATTGATGGTTTACTGCAACTTAGGCTG
GAGGAGCGTGGCGTGCTGGATCATCCTCTCATACGCAAGGTTGAGGAGAT
TGGGACGCACCACATTGCGCTCCACAATGACTTGATCTTGCTCAGGAAGG
AGTACTTCCTGGCAAGTGACTATGATGTTGATTTGCCTTCATCGGAGGCA
AGCAGCACTCTGTTTTTTCTTTTGCAAATGGCTACTTTCATGAAATACTT
TTTAGAGGATCTATGCAGCCATTTTGCCGCTCGCTGCCGGATAATCCCAT
ACAAGAATGTCTCGAGCCTGTGGATGGATCAATCTGGGGCGGTGCTCCAG
AAGAAGCTCTTGAAGCTCGAGTTCACTACGCTCTTTGAGTACCTCCAACG
GCTGTCTCCGACTTCTACATCCCCTGGAACTCCATGGTAA
(SEQ ID NO: 16)
ATGGCTGTTTCATCCATTGCGAGCATTTTCGCAGCAGAGAAAAGCTATTC
CATTCCACCAGTGTGTCAACTCCTTGTCTCTCCAGTGCTGAATCCTCTGT
ACGATGCAAAGGCCGAGTCTCAGATCGATGCGTGGTGCGCGGAGTTTCTG
AAGTTGCAACCTGGAAGCGAGAAAGCTGTGTTTGTTCAAGAAAGCAGGCT
TGGATTGCTCGCGGCTTATGTTTACCCGACCATTCCATACGAGAAGATTG
TTCCGGTTGgaaagttcttcgcttcgttcTTTCTTGCAGATGACATTCTG
GATAGCCCGGAGATCTCCTCGTCGGACATGAGgaatgtggcaactgcata
caAGATGGTTTTAAAGGGAAGATTTGACGAGGCCACGCTTCCAGTGAAAA
ATCCGGAGCTGCTGAGGCAAATGAAGATGTTATCTGAGGTCTTGGAAGAA
TTGTCCCTCCATGTAGTGGATGAATCAGGCCGATTCGTGGATGCTATGAC
CAGGGTGCTCGACATGTTCGAGATTGAATCGAGCTGGCTTCGCAAGCAAA
TCATTCCCAACCTGGATACGTACCTCTGGCTGAGAGAGATCACATCTGGT
GTGGCTCCTTGCTTTGCTCTGATTGATGGTTTACTGCAACTTAGGCTGGA
GGAGCGTGGCGTGCTGGATCATCCTCTCATACGCAAGGTTGAGGAGATTG
GGACGCACCACATTGCGCTCCACAATGACTTGATGTCGCTCAGGAAGGAG
TGGGCGACAGGAAACTACCTCAACGCCGTGCCCATTCTCGCCAGCAATCG
TAAGTGTGGCTTGAACGAGGCAATCGGCAAGGTTGCGAGCATGCTGAAGG
ATTTGGAGAAGGATTTCGCTCGGACAAAGCATGAGATCATTTCAAGTGGG
CTTGCCATGAAGCAAGGAGTCATGGACTATGTGAACGGGATAGAGGTGTG
GATGGCCGGAAACGTAGAATGGGGATGGACGAGCGCTAGATACCATGGAA
TTGGGTGGATCCCTCCTCCAGAAAAATCAGGGACCTTCCAACTCTAG
(SEQ ID NO: 17)
ATGGCTGTTTCATCCATTGCGAGCATTTTCGCAGCGGAGAAAAGCTATTC
CATTCCACCAGTGTGTCAACTCCTTGTCTCTCCAGTGCTGAATCCTCTGT
ACGATGCAAAGGCCGAGTCTCAGATCGATGCGTGGTGCGCGGAGTTTCTG
AAGTTGCAACCTGGAAGCGAGAAACCCGTGTTTGTTCAAGAAAGCAGGCT
TGGATTGCTCGCGGCTTATGTTTACCCGACCATAGATTGTTCCGATGACA
TTCTGGATAGCCCGGAGATCTCCTCGTCGGACATGACGAATGTGGCAACT
GCATACAAGATGGTTTTAAAGGGAAGATTTGACGAGGCCATGCTTCCAGT
GAAAAATCCGGACCTGCTGAGGCAAATGAAGATGTTATCTGAGGTCTTGG
AAGAATTGTCCCTCCATGTAGTGGATGAATCAGGCCGATTCGTGGATGCT
ATGACCAGGGTGCTCGACATGTTTGAGATTGAATCGAGCTGGCTTCGCAA
GCAAATCATTCCCAACCTGGATACGTACCTCTGGCTGAGAGAGATCACAT
CTGGCGTGGCTCCTTGCTTTGCTCTGATTGATGGTTTACTGCAACTTAGG
CTGGAGGAGCGTGGCGTGCTGGATCATCCTCTCATACGCAAGGTTGAGGA
GATTGGGACGCACCACATTGCGCTCCACAATGACTTGATGTCGCTCAGGA
AGGAGCGGGCGACAGGAAACTACCTCAACGCCGTGCCCATTCTCGCCAGC
AATCGTAAGTGTGGCTTGAACGAGGCAATCGGCAAGGTTGCGAGCATGCT
GGAGGATTTGGAGAAGGATTTCGCTCGGACAAAGCATGAGATCATTTCAA
GTGGGCTTGCCATGAAGCAAGGAGTCATGGACTATGTGAACGGGATAGAG
TGTTTTAGAAATTCATATCTAAGCAGTGTTTTCGACCTGAACAAGCAAAT
TGAAATGCACGGTAGATGTGGTAACATCAAACACGCAGCTCAAATCTTCC
ATGCAAGTTGCTGTGATTTTCCTTCATGGGAGGCAAGCAGCACTCTGTTT
TTTCTTTTGCAAATGCCATTTTGCCGCTCGCTGCCGGATAATCCCTGGGC
GGTGCTCCTGAAGAAGCTCTTGAAGCTCGAGTTCACTACACTCTTTGAGT
ACCTCCAACTGACTTCTACGTCCCCTGGAACTCCATGGTAA
(SEQ ID NO: 18)
ATGGAGGCCACTTTGATCTCCAAATTCTCCACTGTCACGCACTTCGAGCT
TCCGCAGCTTCCCAACAACATCCCATTCGCCTACCACCCGCAATCCGCGA
CGATCAGTGCCCAGATCGACGAGTGGATGCTTCGCAAGATGAAGATCACT
GACCAGAGTGCGAGGAAGAAGATGATCCACTCCAAGATGGGACTGTACGC
CTGTATGATGCATCCCAATGCCGAGAGGGAGAAGCTCGTCCTGGCCGGTA
AGAATCTCTGGGCCCTCCTCCTCATGGACGATTTGCTCGAATCCAGCAGC
AAGGAAGAGATGCCTCGGCTCAACACCACCATCTCCAGCCTTGGCAGTGG
AAATTCCGGGGATGGAGCTATCCGGAATCCTGTGCTGCTTCTGTATAAAG
AAGTTCTGGGAGAGCTTCGAGCTGCCATGGAGCCACCTTTGCTGGACCGC
TACTTGCACTGCCTGGCAGCTTCACTCGAAGGCGTCCGGAAGCAAGTCCA
CCACCGAACCAAGAAGAGCGTCCCTGGACCAGAAGAATATAAGTTCACCC
GTCGTGCCAATGGATTCATGGACATCCTCGGGGGCATCATGACCGAGTTC
TGTATGGGAATCCGCCTCAACCAAGCTCAAATCCAGTCTCCAACCTTCCG
GGAGCTCCTCAACTCTGTGTCTGATTACGTCATTCTCGTCAATGACCTGC
TGTCCTTCCGGAAGGAGTTTTACGGTGGCGATTATCACCACAACTGGATC
TCGGTACTCTCGTACCATGGCCCCTCCGGAATCAGCTTTCAGGATGTGAT
TGACCAGCTGTGTGAGATGATCCAAGCAGAAGAGCACTCAATCCTGGCCT
TGCAGAAGAAGATTGCCGACGAAGAAGGTTGCGACTCGGAGCTGACGAAG
TTCGCAAGTGAGCTAGCAATGGTTGCTTCCGGGAGCCTCGTGTGGTCGTA
TCTCTCTGGCCGCTACCATGGCTATGATAATCCACTGATCACTGGGGAGA
TTTTCAGTGGAACATGGCTGCTGCATCCCGTGGCCACCGTCGTCTTACCA
TCCATCAAGGCTCGAGATACATTGCTGGGGCTCAAAGTTCCGGTTCCACT
GCCTTGA
(SEQ ID NO: 19)
ATGGAAGATGTTCTAGTTTCCAGAATTTTGGGTGTCACCCATTTCGAGCT
CCCATTGCTTCCCAACAACATTGCATTTTATTGCCACCCGGAATTCCAAT
CAATCAGCCTCCAAATCGACGAGTGGTTCCTTGACAAGATGAGAATCGCC
GACGAGACTTCCAAGAAGAAGGTGCTGGAGTCCAGGATCGGTTTGTACGC
CTGTATGATGCATCCCCATGCTGAGAGAGAGAAGATTGTGCTGGCCGGGA
AACATCTCTGGGCCGTCTTCCTCCTTGACGATTTGCTGGAATCCAGCGGC
ACACAAGAGATGCCGAAGCTCAACGCCACCATTTCCGACCTTGCCAGTGG
AAATTCCAACGAGGATGTTACAAATCCTGTGTTGGTTCTCTACCGAGAAG
TTATGGAAGAGATCCGGGCTGGTATGGAGCCACCATTGCTGGATCGCTAC
GTGGAGTGCCTGGGAGCTTCACTGGAAGCCGTGAAGGATCAAGTTCACCA
CCGAGCCGAGAAAAGTATCCCTGGAGTGGAAGCTTACAAGCTTGCCCGCC
GTGCCACTGGATTCATGGAAGCTGTCGGCGGTATCATGACCGAGTTCTGT
ATGGGAATCCGCCTCAACGAAAGTCAAATCCAGTCTCCAGTCTTTCGAGA
GCTCCTCAATTCTGTGTCTGATCACGTTGTTCTTGTCAATGATCTCTTGT
CCTTCCGGAAAGAGTTCTATGAAGGTGCTTGTCACCACAACTGGATCTCA
GTTCTCCTGCAGCACAGCCCCAGCGGGACGAGGTTCCAGGATGTCATTGA
TCAGCTCTGCGAGATGATCCAAGAAGAAGAGCTCTCAATCCTGGCATTGC
AGAGGAAGATTTCCAGTAAAGAAAATAGCGACTCGGAGCTGATGAAGTTC
GCAAGGGAGTTCCCAATGGTTGCTTCCGGGAGCCTAGTGTGGTCGTATGT
CACTGGCCGCTACCATGGCTATGGTAATCCGCTGCTGACTGGGGAGATTT
TCAGCGGAACTTGGCTGCTCCATCCCATGGCCACCGTCGTCTTGCCAAAG
TCTACAGTCTTTTCATTAAACCATTTGGTATATTCTCATGTTATTCTTTG
A
(SEQ ID NO: 20)
ATGGAAGATATTCTAGTTTCCAGAATTTCGGGTGTCACCCATTTCGAGCT
TCCATTGCTTCCCAACAACATTGCATTTTATTGCCACCCGGAATTCCAAT
CAATCAGCCTCCAAATCGACGAGTGGTTCCTTGCCAAGATGAGAATCACC
GACGAGACTTCCAAGAAGAAGGTGTTGGAGTCCAGGATCGGTTTGTACGC
CTGTATGATGCATCCCCATGCTGAGAGAGAGAAGATTGTGCTGGCCGGGA
AACATCTCTGGGCCGTCTTCCTCCTTGACGATTTGCTGGAATCCAGCGGC
ACCCAAGAGATGCCAAAGCTCAACGCCACCATCTTCAACCTTGCCAGTGG
AAATTCCAACGAGGATGTCACAAATCCTGTGCTGGTTCTCTACCGAGAAG
TTATGGAAGAGATCCGGGCTGGTATGGAGCCACCATTGCTGGATCGCTAT
GTGGAGTGCCTGGGAGCTTCACTGGAAGCCGTGAAGGATCAAGTTCACCA
CCGAGTCGAGAAGAGTATCCCTGGAGTGGAAGAATACAAGCTTGCCCGCC
GTGCCACTGGATTCATGGAAGCTGTCGGGGGTATCATGACCGAGTTCTGT
ATGGGAATCCGCCTCAACGAAAGTCAAATCCAGTCTCCAGTCTTTCGAGA
GCTCCTCAATTCTGTGTCTGATCACGTTGTTCTTGTCAATGATCTCTTGT
CCTTCAGGAAAGAGTTCTATGAAGGTGCTTGTCACCACAACTGGATCTCA
GTTCTCCTGCAGCACAGCCCCAGAGGGACGAGGTTCCAGGATGCAATTGA
TCAGCTCTGCGAGATGATACAAGAAAAAGAGCTCTCAATCCTGGCCTTGC
AGAGGAAGATTTCCAGCAAAGAACATAGTGACTCGGAGCTGATGAAGTTC
GCAAGGGAGTTCCCAATGGTTGCTTCCGGGAGCCTCGTGTGGTCGTACGT
AACTGGCCGTTACCATGGCTATGGTAATCCGCTGCTGACTGGGGAGATTT
TCAGTGGAACTTGGCTGCTCCATCCCATGGCCACCGTAAATGGATATCAA
ACCATTCTAGTATATTCTCTTATTAATAATACGGAAATAAAATCTATAAT
CTCCACAATATATACAGTTTCCCAAATCGCGAGTTCTGGTTGA
(SEQ ID NO: 21)
ATGAAAGATCTTTTCAGAATTTCAGGTGTCACTCATTTCGAGCTTCCGCT
TCTTCCCAACAACATTCCATTTGCTTGCCACCCGGAATTCCAATCAATCA
GCCTCAAAATCGACAAGTGGTTCCTTGGCAAGATGAGAATCGCCGACGAG
ACTTCCAAGAAGAAGGTGCTGGAGTCCAGGATTGGTTTGTACGCCTGTAT
GATGCATCCCCATGCTAAGAGAGAGAAGCTTGTTCTCGCCGGGAAACATC
TCTGGGCCGTCTTCCTCCTTGACGATTTGCTGGAATCCAGCAGCAAACAC
GAGATGCCTCAGCTCAACCTCACCATCTCCAACCTTGCCAATGGAAATTC
CGACGAGGATTACACAAATCCTCTGCTGGCTCTCTATCGAGAAGTTATGG
AAGAGATCCGAGCTGCCATGGAGCCACCATTGCTGGATCGATACGTGCAG
TGCGTGGGCGCTTCACTGGAAGCCGTGAAGGATCAAGTTCACCGCCGAGC
CGAGAAGAGTATCCCTGGAGTGGAAGAATACAAGCTCGCCCGCCGTGCCA
CTGGATTTATGGAAGCTGTCGGCGGAATCATGACCGAGTTTTGCATCGGA
ATCCGCCTCAGCCAAGCTCAGATCCAGTCTCCAATCTTCCGGGAGCTCCT
CAACTCTGTGTCTGATCACGTCATTCTCGTCAATGACCTGCTGTCCTTCC
GGAAGGAGTTTTATGGTGGCGACTATCACCACAACTGGATCTCGGTTCTC
TTGCACCACAGTCCCCGCGGGACTAGtttccaggacgtagttgaccGCCT
GTGCGAGATGATCCAAGCAGAAGAGCTCTCAATTTTGGCCTTGCGGAAGA
AGATTGCTGACGAAGAAGGAAGCGActcagagctgactaagtttgCAAGA
GAGTTCCCAATGGTTGCTTCTGGGAGCCTAGTGTGGTCGTATGTCACTGG
CCGCTACCATGGTTATGGTAATCCGCTGCTGACTGGGGAAATTTTCAGTG
GAACTTGGCTGCTTCATCCCATGGCCACCGTCGTCTTGCCATCGAAGTTC
AGAATGGATACCATGAGATTCTCTTTAGCTCCAAAAAAACGCGACTCGTT
TCCCTGA
(SEQ ID NO: 22)
ATGGAGGCCACTTTGATCTCcAAAtTCTCCACTGTCACGCACTTCGAGCT
TCCGCAGCTTCCCAACAACATCCCGTTCGCCTACCACCCGCAATCCGCGA
CGATCAGTCCCCAGATCGACGAGTGGATGCTTCGCAAGATGAAGATCACT
GACCAGAGTGTGAGGAAGAAGATGATCCACTCCAAGATCGGACTGTACGC
CTGTATGATGTATCCCAATGCCGAGAGGGAGAAGCTCGTCCTGGCCGGTA
AGAATCTCTGGGCCCTCCTCCTCATCGACGATTTGCTCGAATCCAGCAGC
AAGGAAGAGATGCCTCGGCTCAACACCACCATCACCAACCTTGGCAGTGG
AAATTCCAGGGATGGAGCTATCCGGAATCCTGTGCTGCTTCTGTATAAAG
AAGTTCTGGGAGAGCTTCGAGCTGCCATGGAGCCACCTTTGCTGGACCGC
TACTTGCACTGCCTGGCAGCTTCACTCGAAGGTGTCCGGAAGCAAGTCCA
CCACCGAACCAGAAAGAGCGTCCCTGGACCGGAAGAATATAAGCTCACCC
GTCGTGCCAATGGATTCATGGACATCCTCGGGGGCATCATGACCGAGTTC
TGTATGGGAATCCGCCTCAACCAAGCTCAAATCCAGTCTCCAACCTTCCG
GGAGCTCCTCAACTCTGTGTCTGATTACGTCATTCTCGTCAATGACCTGC
TGTCCTTCCGGAAGGAGTTTTACGGAGGCGATTATCACGACAACTGGATC
TCGGTTCTCTCGTACCATGGCCCCAGGGGGATCAGCTTTCAGGATGTGAT
TGACCAGCTGTGTGAGATGATCCAAGCAGAAGAGCACTCAATCCTGGCCT
TGCAGAAGAAGATTGCCGACGAAGAAGGTTGCGACTCGGAGCTGACGAAA
TTCGCAAGTGAGCTTGCAATGGTTGCTTCCGGGAGCCTCGTGTGGTCGTA
TCTCTCTGGCCGTTACCATGGCTATGATAATCCACTGATCACTGGGGAGA
TTTTCAGTGGAACATGGCTGCTGCATCCCGTGGCCACCGTCGTCTTCCCA
TCCATCAAGGCTCGCCCCTGA
(SEQ ID NO: 23)
ATGTTTGAGGACGTGATGTTGAGCATTCAAAGTCTCATGGATCCCCCACT
GTTTGCTCGCTACATGATTTGCTTGAGAAACTATCTGGATGCTTTGGTGG
AGGACTCCTCTTTGCGATTTGCCAAATCCATTCCAAGCCTCACTAAACAC
CAGCTGCTCCGGAAGCAGTTGGAGGCATTATATAGAGACAAGCACTACAG
CTATCTCTGTGTCATCTTTTGTCACGATAATGCAAGCTTTCAGGGGACCG
TGGACAAAGCATGTGAAATGATCCAGGAGACCGAAGGTGAGATCTTGCAA
CTCCAAAAGAAGCTGATGAAGCTGGGAGAGGAAACTGGGAACAAAGATCT
GGTGGAGTACGCAAGGTACCCTTGTGTTGCATCTAGAAACCTTCGCTGGT
CGTATGTCACACGAACCAGCAGTCGTGAACCATTCCATGCGACCTGGTTT
CTACTTCCAGAAGTCACCCTCATCGTGCCATTCGGATCCAAATGCGGTGA
TCATCCATTTGCCATCACAGAAAATCATTTGGTTTGA
(SEQ ID NO: 24)
ATGGAAGATGTCTTGGCTGAAAGATTGTCTAGAGTTAGCAAGTTTGATTT
GCCTTCCATTCCTTGCAGCATTCCCTTGGAATCTCATCCTGAATTCTCTC
GGATATCTGAAGTTACTGATGCATGGGCTATTAGAATGTTGGGTATCACT
GATCCATATGAGAGACAGAAAGCTATTCAGGCAAGATTCGGTTTGCTCAC
CGCACTAGCTACACCTAGGGGAGAGAGCAGTAAACTCGAGGTTGCATCAA
AGCATTTCTGGACTTTTTTCGTTCTAGATGACATTGCCGAGACAGACTTC
GGTGAGGAAGAGGGGCAAAAAGCTGCTGATATTTTGCTTGAAGTTGCTGA
GGGAAGCTATGTTTTTAGTGAAAAAGAAAAGCAGAAGAATCCGAGCTATG
CCATGTTTGAGGAGGTGATGTCGAGCTTCCGCAGTCTCATGGATCCCCCA
CTGTTTGCTCGCTACATGACTTGTCTGAAAAACTTTCTGGATTCTGTGGT
GGAGGAGGCCTCTTTGCGATTTGCCAAATCCATTCCAAGCCTTGAGAAAT
ACCAGCTGCTCCGGAGAGAGACAGTCTTTGTTGAAGCGTCCGGAGGTATT
ATGTGTGAGTTTTGCATGGATCTCAAGCTGGATAAGGGTGTGGTTGAATC
CCCAGAATTCGTAGCCTTTGTCAAGGCAGTGGTTGATCACGCCGCCCTTG
TCAATGATCTCCTTTCCTTCCGACACGAGATGAAAATCAAGTGCTTCCAC
AACTATCTCTGTGTCATCTTTTTCCACAGCCCGGATAATGCAAGTTTTCA
AGAGACTGTCGACAAGGTATGCAAAATGATCCAGGAGACCGAAGCTGAGA
TTTTGCAACTCCAAAAGAAGGTGATGAAGATGGGCGTGGAAACTGGGAAC
AAAGATCTAGTGGAGTACGCAACATGGTATCCTTGTTTTGCATCTGGACA
CCTTCGCTGGTCGTATGTCACAGGACGCTACCATGGACTTGACAATCCAC
TGCTGAATGGTGAACCATTCCATGGGACCTGGTTTTTACATCCAGAAGTC
ACTCTCATGTTGCCATTTGGAGCCAAATGTGGTGATCATCCATGGATTGC
AAGAAGCTAG
(SEQ ID NO: 25)
ATGGAAGATGTTTTGGCTGAAAAATTGTCAAGAGTTTGCAAGTTCGATTT
GCCATTCATCCCTTGTAGCATTCCCTTTGAATGCCATCCTGATTTTACTA
GGATATCCAAAGATACTGATGCATGGGCTCTTAGAATGTTGAGTATCACT
GATCCATATGAGAGAAAGAAAGCTCTTCAGGGAAGACATAGCTTGTATAG
CCCAATGATTATTCCAAGAGGGGAGAGCAGCAAAGCGGAGCTTTCATCAA
AGCATACATGGACTATGTTTGTTTTAGATGACATTGCCGAGAATTTTAGT
GAGCAAGAGGGAAAAAAAGCTATTGATATTCTTCTTGAAGTTGCTGAGGG
AAGCTATGTCTTAAGCGAAAAAGAGAAGGAGAAGCATCCTAGCCACGCCA
TGTTTGAGGAAGTGATGTCGAGCTTTCGGAGTCTCATGGATCCCCCACTG
TTTGCTCGCTACATGAATTGCTTGAGAAACTATCTGGATTCTGTGGTGGA
GGAGGCCTCTTTGCGAATTGCCAAATCTATTCCAAGCCTCGAGAAGTACC
GGCTGCTCCGGAGAGAGACAAGCTTTATGGAAGCAGACGGAGGCATTATG
TGTGAGTTTTGCATGGATCTCAAGTTGCATAAGAGTGTGGTGGAATCCCC
AGACTTCGTAGCCTTTGTCAAGGCAGTGATTGATCACGTcgttcttgtca
atgatctccTTTCCTTCCGACACGAGCTGAAAATCAAGTGCTTCCACAAC
TATCTCTGTGTCATCTTTTGCCACAGCCCGGATAATACAAGCTTTCAAga
gactgtggacaaggtatgCGAAATGATCCAGGAGGCCGAAGCCGAGATCT
TGCAACTCCAACAGAAGCTGATTAAGCTGGGCGAGGAAACTGGGGACAAA
GATCTGGTGGAGTATGCAACATGGTACCCTTGTGTGGCATCTGGAAATCT
TCGCTGGTCATACGTCACAGGACGCTACCATGGACTTGACAATCCGCTGC
TGAATGGTGAACCATTCCAAGGAACCTGGTTTCTACATCCAGAAGCCACC
CTCATCTTGCCATTGGGATCCAAATGCGGCAATCATCCATTTATCATGAT
TAAGGGTCATCATCACCATCACCATTGA
(SEQ ID NO: 26)
ATGGAAGATGTTTTGGCTGAAAAATTGTCAAGAGTTTGCAAGTTCGATTT
GCCATTCATCCCTTGTAGCATTCCCTTTGAATGCCATCCTGATTTTACTA
GGATATCCAAAGATACTGATGCATGGGCTCTTAGAATGTTGAGTATCACT
GATCCATATGAGAGAAAGAAAGCTCTTCAGGGAAGACATAGCTTGTATAG
CCCAATGATTATTCCAAGAGGGGAGAGCAGCAAAGCGGAGCTTTCATCAA
AGCATACATGGACTATGTTTGTTTTAGATGACATTGCCGAGAATTTTAGT
GAGCAAGAGGGAAAAAAAGCTATTGATATTCTTCTTGAAGTTGCTGAGGG
AAGCTATGTCTTAAGCGAAAAAGAGAAGGAGAAGCATCCTAGCCACGCCA
TGTTTGAGGAAGTGATGTCGAGCTTTCGGAGTCTCATGGATCCCCCACTG
TTTGCTCGCTACATGAATTGCTTGAGAAACTATCTGGATTCTGTGGTGGA
GGAGGCCTCTTTGCGAATTGCCAAATCTATTCCAAGCCTCGAGAAGTACC
GGCTGCTCCGGAGAGAGACAAGCTTTATGGAAGCAGACGGAGGCATTATG
TGTGAGTTTTGCATGGATCTCAAGTTGCATAAGAGTGTGGTGGAATCCCC
AGACTTCGTAGCCTTTGTCAAGGCAGTGATTGATCACGTCGTTCTTGTCA
ATGATCTCCTTTCCTTCCGACACGAGCTGAAAATCAAGTGCTTCCACAAC
TATCTCTGTGTCATCTTTTGCCACAGCCCGGATAATACAAGCTTTCAAGA
GACTGTGGACAAGGTATGCGAAATGATCCAGGAGGCCGAAGCCGAGATCT
TGCAACTCCAACAGAAGCTGATTAAGCTGGGCGAGGAAACTGGGGACAAA
GATCTGGTGGAGTATGCAACATGGTACCCTTGTGTGGCATCTGGAAATCT
TCGCTGGTCATACGTCACAGGACGCTACCATGGACTTGACAATCCGCTGC
TGAATGGTGAACCATTCCAAGGAACCTGGTTTCTACATCCAGAAGCCACC
CTCATCTTGCCATTGGGATCCAAATGCGGCAATCATCCATTTATCACGAT
TTGA
(SEQ ID NO: 27)
ATGGAGTTTCTCTTGGGAAAGATTGTCCCTCGTTTCGAGTTGCCTCTTCT
TCCAAACAACATCCCCTGTGCTTGCCACCCGGATTCCTCCTCTCTTAGCC
AGGAACTCGATGAATGGTTCATTTCCAAGTTAGGCATCACTGACGAGAGC
GCCCAGAAGAAGATCGTCCAGTCGAGAATCATGATCTTTGCTTGCTTGAT
GCATCCCAATGGCGAGAGGGACAGAGTCCTCCTGGCAGGGAAGCATTTGT
GGGTGTGCTTCTTGGTGGATGACATCCTCGAGTCAAGCACCAGGGAAGCC
TATGGCAGCCTCAAATCCATCGTCTGGAGCATTGCCACCACTGGAATCTA
CAAAGCATCCAATGAGGAGCATGATCATTGTCTCGTGCTGCTGCTCTACC
AGGAAGTTTTGGCGGAACTCCGCAAGAAAATGCCCAGTTCTTTATTCACT
CGCTATTGCAAGATCCTCTCAAGCTACCTGGATGGCGTCGAGGAGGAGGT
CAAACACCAGGTGAAGAACACGATCCCGAGCAGCGAGGAGTATCGGCTCC
TTCGCCGCCGCACTGGATTCATGGAGGTGATGGCGTGCATCATGACCGAG
TTCTGCGTTGGAATCAAGCTCGAGGAGTCGGTTGTAAACTTGGGAGAGAT
CCGTAAGCTCGTCAAGGTCATGGACGACCACATTGTCATGGTGAACGACC
TCCTGTCACTTCGCAAGGAGTATTACAGCAGCACCATTTGCCATAACTGG
GTGTTTGTTTTGCTTGCGGATGGCTGTGGCACTTTTCAGGAGAGTGTGGA
TCATGTTTGCGAGATGATTAAGCAGGAGGAGGGTTCGATTCTGGATTTGC
AGCAGAAACTTATTATCAAGGCAAAGGTGGACAAAAATCCGGAGCTTCTC
AAATTTGCATGTAATGTTCCAATGGCAGTTGCTGGTCATCTAAAGTGGTC
TTTCATTACGGCTCGTTACCATGGGTGTGACAATGCTTTGCTCAATGGTG
AGTTGTTTCATGGAACTTGGCTCATGGATCCCAATCAAACAATAATCCAG
AAAAACATATAG
(SEQ ID NO: 28)
ATGGCTGTTTCATCCATTGCGAGCATTTTCGCAGCAGAGAAAAGCTATTC
CATTCCACCAGTGTGTCAACTCCTTGTCTCTCCAGTGCTGAATCCTCTGT
ACGATGCAAAGGCCGAGTCTCAGATCGATGCGTGGTGCGCGGAGTTTCTG
AAGTTGCAACCTGGAAGCGAGAAAGCTGTGTTTGTTCAAGAAAGCAGGCT
TGGATTGCTCGCGGCTTATGTTTACCCGACCATTCCATACGAGAAGATTG
TTCCGGTTGGAAAGTTCTTCGCTTCGTTCTTTCTTGCAGATGACATTCTG
GATAGCCCGGAGATCTCCTCGTCGGACATGAGGAATGTGGCAATCGCATA
CAAGATGGTTTTAAAGGGAAGATATGACGAGGCCACGCTTCCAGTGAAAA
ATCCGGAGCTGCTGAGGCAAATGAAGATGTTATCTGAGGTCTTGGAAGAA
TTGTCCCTCCATGTAGTGGATGAATCAGGCCGATTCGTGGATGCTATGAC
CAGGGTGCTCGACATGTTTGAGATTGAATCGAGCTGGCTTCGCAAGCAAA
TCATTCCCAACCTGGATACGTACCTCTGGCTGAGAGAGATCACATCTGGT
GTGGCTCCTTGCTTTGCTCTGATTGATGGTTTACTGCAACTTAGGCTGGA
GGAGCGTGGCGTGCTGGATCATCCTCTCATACGCAAGGTTGAGGAGATTG
GGACGCACCACATTGCGCTCCACAATGACTTGATGTCGCTAAGGAAGGAG
TGGGCGAGTGGAAACTACCTCAACGCCGTGCCCATTCTCGCCAGCAATCG
TAAGTGTGGCTTGAACGAGGCAATCGGCAAGGTTGCGAGCATGGTGGAGG
ATTTGGAGAAGGATTTCGCCCAGACAAAGCATGAGATCATTTCAAGTGGG
CTTGCCATGAAGCAAGGAGTCATGGACTATGTGAACGGCATAGAGGTGTG
GATGGCCGGAAACGTAGAATGGGGATGGACGACCGCTAGATACCATGGAA
TTGGGTGGATCCCTCCTCCAGAAAAATCAGGGACCTTCCAACTCTAG
(SEQ ID NO: 29)
ATGGAGTGTCTCATGGCAAAGCTTGTCCCTCGCCTTGAGTTGCCTCTTCT
TCCAAATAACATCCCCTCTGCTTGCCACTGGGATTCTTCTTCTCTCAGCC
AAGAGCTCGATCAATGGCTCATCTCCAAGCTCGGTATCACCGACGAGAGT
GCCAAGAGGAAGATTGTCCAGTCGAGAGTCATGCTCTTAGCTTGTTTGAT
GCATCCCAATGGCGAGAGGGACAGAGTCCTCTTGGCAGGGAAGCATTTGT
GGGTGTACTTCCTGGTGGATGACATCCTCGAGTCAAGCAGCCGGGAAGGT
TATGGCGCCCTCAAATCCATCGTCTGGAGCATTGCCACCACTGGAATCTA
CAAAGCATCTGAGGAGCATGATCATCATGACCTCGTGCTGCTTCTCTTGG
TGGAAGTCATGGTGGAACTCCGCAAGGAAATGCCCACTTCTTTATTCGCT
CGCTACTGCAAGATCCTCTCAATCTATCTGGATAGCGTCCAGGAGGAGGT
CAAGCACCAAATCAATAACACGATCCCGAGCAGCGAGGAGTACCGGCTTC
TCCGCCGCCGCACTGGATTCATGGAGGTGATGGCCTGCATCATGACTGAA
TTTTGCGTGGGAATCAACCTCGAGGAATTGGTTGTAAACTTGGGAGAGAT
CCGTGAGCTCGTCAAGATCATGGACGACCACATTGTCACGGTGAACGACC
TCTTGTCACTGCGCAAGGAGTATTACAATGGCACCATTTACCACAACTGG
GTAATTGTTTTGCTTGCCCATGATTGTGCAACTTTTCAGAAGAGTGTGGA
TCGCGTTTGTGAGATGATCAAGCAGGAGGAGGACTCGATTCTAGATTTGC
AGAAGAAACTTATCATCAAGGCAAAGGTGGACAAGAACCCAGAGCTTCTC
AAATTTGCATTTAATGTTCCAATGGCTGTGGCTGGTCATCTAAAGTGGGC
TTTTATTACTGCTCGTTACCATGGTTGTGACAACGCTTTGCTCGATGGTG
AGTTGTTTCATGGAACTTGGATCATGGACCCCAATCAAACGGTAATCGTG
AAAAACATGTAG
(SEQ ID NO: 30)
ATGGCTCCCTACGATTTCGTTCCAAATGTGCAGTGTTCGTTCCCTGTGAA
GTGCCACCCTCTGTATTCTTTCATTCGTCCACGCTTGGAAGATTGGGCTG
CAACTTTGGAGCCTGGGCATGGTGAAGGGAACCCCAAAAGTAGGAAATGC
CTTGTTGCTGAGAAAAGAGTTCTAGCCACTTGCATGTTGATCCCTGTTGC
TGATGATGCCAGAATTGAGAATATGTGCAAGCTTGCCTGTTGGTGCTTCC
ATGTTGATGATATCCTTGACGACTTGCAGGGCCTGGGAGCTGACTCGGGA
GGTGCCAAGAGGCTTGTTGATAGCTACCTTGGCATAATCCGTGCGTGGCA
GATATGGAATTTCCACGgttctgtgatatgtggaatgatctgcgtgcagA
TATGCCACTCAAGCAGTACCAGCGATTTGCCAACAGAGTGTCCGAGCTGT
TGGAGGCAAGCGTGAATCAGGTGAAGCTAAGGAATCTGAAAACGGTGATG
GGCTTGGAGGAGCTGCTGGCTCACCGTCGCGTGTTAGTTGGTGTATTTGT
TATGGAAACTCTAATGGAGTATGGCATGGGATTCGAACTCCAGGACGACG
CCATTTCAAATCAGGACCTCCAAGAGGCTGAAAGTCTGGTTGCAGACCAC
GTAAGTTGTGTCAACGACTTGTTTTGCTTCCTGGTGGACAGTGCAACTGG
CGATACTCAGTTCAATATCGTCCACACGATCATGTGCGGCAATTCGGATT
CAAAGGGCTTTTCTTTCGAGTATGCAGCCGACAAAGTTCACAAACTGGTG
CAGAGTATCGAGCATCGATTCAAGAAGCTGTGTGAGAATATCAGAAGATC
AAGCTGCTACAATGGTGCAATGGAGGCTTACCTGGAAGGCTTGTCTCATA
TTATATCCGGCAACCTTGAGTGGCACCGGCAGACAGGACGATACAAACTG
GTATCTTGA
(SEQ ID NO: 31)
ATGGGGATGCTTAACGATGTCTACACTGATCTAAAGGGTTTCATGAAGCC
TGGACATAAGACCCGGTTTTCGAATTCCATGATTGATGTGCTCGACATGT
TTGAGGTGGAATCAAGTTGGCTTCACAAGAAACTGGTTCCCAACTTTGAG
ATTTATATGTGGATGAGGGAGGTAACGGCTGGGGTTATCCCTTGCATGGT
GGCAATAGACTTCCTTAATAATTTTGGGCTGGAAGAGGAAGGAATGCTAG
ACGATCTCCATATTCAAACGCTGGAAGTGATTGCAAATCGCCATTCGTTC
CTAGCCAACGACATGGTCTCCTTCAAAAAGGAATGGGCTTGTGAACAGTA
CCTCAATTCTGTGGCACTGGTTGGTTACAGTAGCAACTGTGGCTTAAACG
AGGCAATGGAGAAAGTTGCAGAAATGGTTCAGGATTTGGAGAAAGAGTTT
GCTGACATCAAACAAAAGGTTCTGTCAAACAAGGACTTGAACAAGGGAAA
TGTCATGGGGTATGTGCAAGGCTTGGAGTATTTCATGGCTGGAAATATAG
AGTTTAGCTGGCTCTCTGCGAGATATCATGGGGTGGGATGGGTTTCACCA
GCTGAGAAATATGGTACCTTGGAGTTCTAG
(SEQ ID NO: 32)
ATGGCAAGTCCGTGTTTACAGAAGCTACCAGCTGTAGAACATCTCTTTGC
TCTCACGAGGTTCGAGCTTCCGGAGATACCATGCTCTCTATCTTTCCAAA
GGCATCCCGAGTATATGTCAATCACTAAGGAAGCAAACGAGTGGGCATTC
AAATGCATGAGGAGGGATTTCAGTCCGGAGGAGAAGAAATGCCTGGTCCA
GTGGAAAGTTCCAATGTTTACGTGCCTCTCCACGCCTCACGCTCCGAAAG
CGAACATGGTGGCGTCGGCCAAGTTTGCCTGGCTCACTGCCTTCCTGGAC
GATCCGTTTGATGACAACGAGGTTGCTGGGGGAGCTCTCGCGACATCGTA
TCTCGACACTGTTCTTAGCCTCTGCTACGGAACCGCCTCGCTCGCGGAAA
TTCCGGACATTCTTGCCTATCGAGCGTGCCACGATCTGATGAAGGATTTG
AGGTCTCTGTTGAAGCCGAAGCTGTTTAAGCGCACGGTTTCCACTGTTGA
GGGCTGGGCGAGAAGCATCTCGAGTGACGACTTGACGCAGGACTACGAGC
TCTACCGGAGGAAGAACGTCTTCATTCTGCCACTCATCTACGCAATGGGT
GCTTCGTTTGACGATGAGGATGTCGAGTCCCTGGATTACATCAGGGCTCA
GAACGCTATGCTCGATCATATGTGGATGGTGAACGACGTCTTCTCGTTTC
CCAAGGAGTTTTACAAGAAGAAGTTTAACAATCTTCCGGCGGTGCTGCTG
CTCACCGATCCGAGCGTGCAGACGTTTCAGGACGCCGTGAACACTACGTG
CAGGATGATCCAGGACAAGGAAGACGAATTCATCTATTACCGTGATATCC
TTGCCACGAATGCGTCCCGGAATGGGAAGAAAGATTTCCTGAAGTTCCTG
GATGTTCTCTCCTGCGCAATCCCAGCGAATCTGGTGTTCCATTATGCGAG
CAGCCGCTACCATGGCATGGATAACCCCCTACTGGGTGGACCCACGTTTA
GTGGGACCTGGATTCTGGATCCAAAGCGCACCATCATCTTGTCGGACCCG
AAAAGGTGGAACGTGGTGGCAAGTTCAAACAAACTCAACCAGATCCAAAA
TTTATCAAACTTGATATGA
(SEQ ID NO: 33)
ATGGGTGCTTTATTTGACGATGAGGATGTTGAATCCCTGGATTACATCAG
TGCTCAGAACGCTATGCTCGATCATATGTGGATGGTGAACGACGTCTTCT
CGTTTCTCAAGGAGTTTTACAAGAATAAGTTTAACAATCTTCCGGCGGTG
CTGCTCACCGATCAGAGCGTGCAGACGTTTCAGGACGCCGTGAACACTAC
GTGGAGGATGATCCAGGACAAGGAAGACGAATTCATCTTTTACCGCGATA
TCCTTGCCGCGAATGCGTCCCGGAATGGGAAGAAAGATTTCCTGAAGTTC
CTGGATGTTCTCTCCTGCGCAATCCCAGCGAATCTGGTGTATGCGAGCAG
CCACTACCATGGCGTGGATAACCTACTGAGTGGAGGCACGTTTCGTGGGA
CTTGGATTCTGGATCCAAAGCGCACCATCATCGTGTCGGACCCGAAAAGT
TGCAATGTGGTGGCAACTACGGACGAAGTGAAAATCAATGTTTCATATGC
ATGGCTATTTGTCATTCTAATTCTTGCAAACTGA
(SEQ ID NO: 34)
ATGCCAGGGGAGTACAGCTTCTACAACTTCCTTGACATGGGAGTCGCGCC
TTACGGCGATTACTGGAAGAACATGCGGAAGCTGTGCGCCACAGGCACCA
TTCCCAGCCGGAGAGAGAAGATCGGTCCATACTTGTTGGACAGTGCAAGG
AGAGAGAGATGGGGTTTCCTCCCAAAGAGGTccgatcaatggaagagacc
cactgggtgaaaactgtcaagttgagacggcggtgccctccacctggaat
tccccagacgagaacatagatgaagagcaatcgcgcatgctagaaacccc
atgttgaattacgccacaaacgacgtcttgatatcattttattcattcat
tcattatattctctcagaacaaaagagagtagtccttttttcagtgaata
attgaccggtctattctttgccaggtGTGATTTGACAACCACCGGTTCAA
CCTGCACAATCAAAgtcagtccaagcaagacagcatgatcaagtgaaact
tagcaatataaattgaagagctctgaaatatctcgaaaccatctctaaag
aaatggcaagtccgtgtttacagAAGCTACCAGCGGTAGAACATCTCTTT
GCTCTCACGAGTTTCAAGCTTCCGGAGATACCATGCTCTCTATCTTTCCA
AAGGCATCCGGACTATATGTCAATCACCAAGGAAGCAAACGAGTGGGCAT
TCAAATGCATGAGGAGGGATTTCAGTCCGGAGGAGAAGAAATGCCTGGTC
CAGTGGAAAGTTCCAATGTTTACGTGCCTCTCCACGCCTCACGCTCCGAA
AGCGAACATGTTGGCATCGGCCAAGTTTGCCTGGCTCACTGCCTTCCTGG
ACGATCCGTTTGATGACAACGAGGTTGCTGGGGGAGCTCTCGCGACATCG
TATCTCGACACTCTTCTTAGCCTCTGCTATGGAACCGCATCGCTCGCGGA
AGTTCCGGACATTCTTGCCTATCGAGCGTGCCACGATCTGATGGAAGATT
TGAGGTCTCTGTTTAAGCCGGAGCTGTTCAGGCGCACGGTTTCCACTGTT
GAGGGCTGGGCGAGAAGCATTTTGAGTGACGACTTGACGCACGAGTACGA
GCTCTACCGGAGGACTAACGTCTTCATTTTGCCACTCATCTACGCAATGG
GTGCTTCGTTTGACGATGAGGATGTCGAGTCCCTGGATTACATCAGGGCT
CAGAACGCTATGCTCGATCATATGTGGATGGTGAATGACGTCTTCTCGTT
TCCCAAGGAGTTTTACAAGAAGAAGTTTAACAATCTTCCGGCCGTGCTGC
TGCTCACCGATCCGAGCGTGCAGACGTTTCAGGACGCCGTGAACACTACG
TGCAGGATGATCCAGGACAAGGAAGACGAATTCATCTATTACCGCGATAT
CCTTGCCAGCGTCCCGGAATGGGAAGAAAGCTTTCCTGAAGTTCCTGGAT
GTTCTCTCCTGCACAATCCCAGCGAATCTGGTGTTCCATTATGCGAGCAG
CCGCTACCATGGCATGGATAA
(SEQ ID NO: 35)
ATGGGGAGCCTGTGTTTGCAGAAGCTATCAGCGGTAGAACGTCTCTTCGC
TCTCGAGAGTTTCGAGCTTCCGGAGGTACCATGCTCTCTCTCTTTCCACA
GGCACCCCGAGTACAAGTCAATCACCAGGGAAGCAAACGAGTGGGCATTC
AAATGCACGAGGAGGGATTTGAGTCCGGAGGAGAAGAAATCCCTGCTCCA
GTGGAAGGTTCCAATGGTGACATGCCTTTCCACGGCTCACGCTCCGAAAG
AGAATATGGTGGCGTCGGCCAAGTTTGCTTGGGCCATTGCCTTCCTGGAC
GACCCGATTGATGACAACGAGGTCGCCGCAACGTCGTATCTCGACACTGT
TCTTAGCCTCTGCAATGGAACCGCATCGCTTGCGGAAGTTCCAGACATTG
TTGCGTATCGAGCTTGCCACGATCTGATGAAGGATTTGAGGTCTCTGTTG
CAGCCGGAGCTCTTCAAGCGCACAGTTTCCACTGTCGAGGGTTGGGCGAG
AAGCATCTCGAGTGACGACTTGAAGCAGGACTACAAGCTCTACAGGAGGA
ACAACATCTTCATTCTGCCACTGTTCTACACACTCATTGGCGCTTCCTTT
GAAGATGAGGATGTCGAGTCCCCGGATTTCGTCAGTGCTCAGAACGCTAT
GCTTGATCATATATGGATGGTGAACGATATCTTCTCGTTTCGCAATGAGT
TCTACAAGAAGAAGTTGAACAACCTGCCGGCTGTGCTGCTGCTCACCGAT
CCGAGCGTGCAGACGTTTCAGGAAGCAGTGAACGCTACATGCAGGATGAT
CCAGGACAAGGAAGAAGAATTCATCTATTACCGCAACATCCTTGCCGCGA
ACGCGTCCCGGAATGGGAAGGACTTCTTGAAGTTCCTGGATGTTCTCTCC
TGCGCAATCCCGGCGAATCTGGCGTTCCACTATGCGAGCAGCCGCTACCA
CGGCATGGATAACCCTCTTCTGGCCGGGGGCACGTTTCATGGGACCTGGA
TTCTGGATCCAAAGCGCACCATCATCGTTTCGGACCCGAACAGAAGTAAC
GGAGCGGCATCAAACAAACTCAACCATATCCAAGATTTATCAAAGTTGAT
ATGA
(SEQ ID NO: 36)
ATGGCCGTATATAAGCAGGGTAGCGGATTCAAAACCGAGGCATCCGTAAT
TTTGGGTGTCACCCATTTCGAGCTCCCATTGCTTCCCAACAACATTGCAT
TTTATTGCCACCCGGAATTCCAATCAATCAGCCTCCAAATCGACGAGTGG
TTCCTTGACAAGATGAGAATCGCCGACGAGACTTCCAAGAAGAAGGTGCT
GGAGTCCAGGATCGGTTTGTACGCCTGTATGATGCATCCCCATGCTGAGA
GAGAGAAGATTGTGCTGGCCGGGAAACATCTCTGGGCCGTCTTCCTCCTT
GACGATTTGCTGGAATCCAGCGGCACACAAGAGATGCCGAAGCTCAACGC
CACCATTTCCGACCTTGCCAGTGGAAATTCCAACGAGGATGTTACAAATC
CTGTGTTGGTTCTCTACCGAGAAGTTATGGAAGAGATCCGGGCTGGTATG
GAGCCACCATTGCTGGATCGCTACGTGGAGTGCCTGGGAGCTTCACTGGA
AGCCGTGAAGGATCAAGTTCACCACCGAGCCGAGAAAAGTATCCCTGGAG
TGGAAGCTTACAAGCTTGCCCGCCGTGCCACTGGATTCATGGAAGCTGTC
GGCGGTATCATGACCGAGTTCTGTATGGGAATCCGCCTCAACGAAAGTCA
AATCCAGTCTCCAGTCTTTCGAGAGCTCCTCAATTCTGTGTCTGATCACG
TTGTTCTTGTCAATGATCTCTTGTCCTTCCGGAAAGAGTTCTATGAAGGT
GCTTGTCACCACAACTGGATCTCAGTTCTCCTGCAGCACAGCCCCAGCGG
GACGAGGTTCCAGGATGTCATTGATCAGCTCTGCGAGATGATCCAAGAAG
AAGAGCTCTCAATCCTGGCATTGCAGAGGAAGATTTCCAGTAAAGAAAAT
AGCGACTCGGAGCTGATGAAGTTCGCAAGGGAGTTCCCAATGGTTGCTTC
CGGGAGCCTAGTGTGGTCGTATGTCACTGGCCGCTACCATGGCTATGGTA
ATCCGCTGCTGACTGGGGAGATTTTCAGCGGAACTTGGCTGCTCCATCCC
ATGGCCACCGTCGTCTTGCCAAAGTCTACAGTCTTTTCATTAAACCATTT
GGTATATTCTCATGTTTGA
(SEQ ID NO: 37)
ATGGCAAGTCCGTGTTTACAGAAGCTACCAGCGGTAGAACATCTCTTTGC
TCTCACGCCGGAGATACCTTTCCAAAGGCATCCCGAGTATATGTCAATCA
CCAAGGAAGCAAACGAGTGGGCATTCAAATGCATGAGGAGGGATTTCAGT
CCGGAGGAGAAGAAATGCCTGGTCCAGTGGAAAGTTCCAATGTTTACGTG
CCTCTCCACGCCTCACGCTCCAAAAGCCAACATGGTGGCGTCGGCCAAGT
TTGCCTGGCTCACTGCCTTCCTGAACGATCCGTTTGATGACAACGAGGTT
GCTGCGGGAGCTCTCGCGACATCGTATCTCGACACTGTTCTTAGCCTCTG
CTATGGAACCGCATCGCTCGCGGAAGTTCCGGACATTCTTGCCTATCGAG
CGTGCCACGATCTGATGGAGGATTTGAGGTCTCTGTTGAAGCCGGAGCTG
TTCAAGCGCACGGTTTCCACTGTTGAGGGCTGGGCGAGAAGCATCTCGAG
TGACGACTTAACGCAGGACTACGAGCTCTACCGGAGGAAGAACGTCTTCA
TTCTGCCACTCATCTACGCAATGGGTGCTTCGTTTGACGATGAGGATGTT
GAGTCCCTGGATTACATCAGGGCTCAGAACGCTATGCTCGATCATATGTG
GATGGTGAACGACGTCTTCTCGTTTCCCAAGGAGTTTTACAAGAAGAAGT
TTAACAATCTTCCGGCGGTGCTGCTGCTCACCGATCCGAGCGTGCAGACG
TTTCAGGACGCCGTGAACACTACGTGCAGGATGATCCAGGACAAGGAAGA
CGAATTCATCTATTACCGTGATATCCTTGCCACGAATGCGTCCCGGAATG
GGAAGAAAGATTTCCTGAAGTTCCTGGATGTTCTCTCCTGCACAATCCCA
GCGAATCTGGTGTTCCATTATGCGAGCAGCTGCTACCATGGCATGGATAA
CCCCCTACTGGGTGGAGGCACGTTTCGTGGGACTTGGATTCTGGATCCAA
AGCGCACCATCATCGTGTCGGACCCGAAAAGCCAAGCCATTCCTCACGCG
GTGCACATATGGAAGTCAGCAGTATTCTATGCTCAGTCTTATTTCATCCA
GAGCTTAGAAGACTAG
(SEQ ID NO: 38)
ATGGCAAGTCTGTGTTTACAGAAGCTACCAGCGGTAGAACATCTCTTTGC
TCTCACGAGATTCGAGCTTCCGGAGATACCATGCTCTCTATCTTTCCAAA
GGCATCCCGAGTATACGTCAATCACCAAGGAAGCGAACGAGTGGGCATTC
AAATGCATGAGGAGGGATTTCAGTCCGGAGGAGAAGAAATGCCTGGTCCA
GTGGAAAGTTCCAATGTTTACGTGCCTCTCCACGCCTCACGCTCCGAAAG
CAAACATGGTGGCGTCGGCCAAGTTTGCCTGGCTCACTGCCTTCCTGGAC
GATCCGTTTGATGACAACGAGGTTGCTGGGGGAGCTCTCGCGACATCGTA
TCTCAACACTGTTCTTAGCCTCTGCTATGGAACCGCATCGCTCGCGGAAG
TTCCGGACATTCTTGCCTATCGAGCGTGCCACGACCTGATGAAGGATTTG
AGGTCTCTGTTGAAGCCGGAGCTGTTCAAGCGCACGGTTTCCACTGTTGA
GGGCTGGGCGAGAAGCATTTTGAGTGACGACTTGACGCAGGACTACGAGC
TCTACCGGAGGAAGAACGTCTTCATTCTGCCACTCATCTACGCAATGGGT
GCTTCGTTTGACGATGAGGATGTCGAGTCCCTGGATTACATCAGGGCTCA
GAACGCTATGCTCGATCATATGTGGATGGTGAACGACGTCTTCTCGTTTC
CCAAGGAGTTTTACAAGAAGAAGTTTAACAATCTTCCGGCGGTGCTGCTG
CTCACCGATCCGAGCGTGCAGACGTTTCAGGACGCCGTGAACACTACGTG
CAGGATGATCCAGGACAAGGAAGACGAATTCATCTATTACCGCGATATCC
TTGCCACGAATGCGTCCTGGAATGGGAAGAAAGATTTCCTGAAGTTCCTG
GATGTTCTCTCCTGTGCAATCCCAGCGAATCTGGTGTTCCATTATGCGAG
CAGCCGCTACCATGGCATGGATAACCCCCTACTGGGTGGAGGCACGTTTC
GTGGGACTTGGATTCTGGATCCAAAATGCACCATCATCGTGTCGGACCCG
AAAAGGTGCAACGTGGTGGCAAGTTCAAACAAACTCAACCAGATCCAAAA
TTTATCAAACTTGATATGA
(SEQ ID NO: 39)
ATGCCAGGGGAGTACAGCTTCTACAACTTCCTTGACATGGGATTCGCGCC
TTACGGCGATTACTGGAAGAACATGCGGAAGCTGTGCGCCACAGGCACCA
TTCCCAGCCGGAGAGAGAAGATCGGTCCATACTTGTTGGACAGTGCAAGG
AGAGAGAGATGGGGTTTCCTCCCAAAGAGGTGTGATTTGACAACCACCGG
TTCAAATATTTTCCCTACACAATCAAACCTCTGCTATGGAACCGCATCGC
TCGCGGAGGTTCCGGATATTCTTGCCTATCGAGCGTGCCACGATCTGATG
AAGGATTTGAGGTCTCTGTTGAAGACGGAGCTGTTCAGGCGCACGGTTTC
CACTGTTGAGGGCTGGGCGAGAAGCATTTTGAGTGACGACTTGACGCAGG
ACTACGAGCTCTACCGGAGGAAGAACGTCTTCATTCTGCCACTCATCTAT
GCAATGGGTGCTTCGTTTGACGATGAGGATGTCGAGTCCCTGGATTACAT
CAGGGCTCAGAACGCTATGCTCGATCATATGTGGATGGTGAACGACGTCT
TCTCGTTTCCCAAGGAGTTTTACAAGAAGAAGTTTAACAATCTTCCAGCG
GTGCTGCTGCTCACCGATCCGAGCGTGCAGACGTTTCAGGACGCCGTGAA
CACTACGTGCAGGATGATCCAGGACAAGGAAGACGAATTCATCTATTACT
GCGATATCCTTGCCAGCGTCCCGGAATGGGAAGAAAGCTTTCCTGAAGTT
CCTGGATGTTCTCTCCTGCGCAATCCAGCGAATCTGGTGTTCCATTATGC
GAGCAGCCGCTACCACATGGATAACCCCCTGGGTGGAGGCACGTTTTGTG
GGACTTGGATTCTGGATCCAAAGCGCACCATCATCATGTCGGACCCGAGA
AGGTGCAACGTGGTGGCAAGTTCAAACAAACTCAACCAGATCCAAAATTT
ATCAAACTTGATATGA
(SEQ ID NO: 40)
ATGGCTCCAGCTCTAGAGAAGATCTATGCTGTTGCTAGGCCTCAAGAATT
TCCATCTCCCAAAGATACCCAGCTTGATTCCTCCAGCGCTTATGCATCCA
GCAACAAGTTCATCACCCGCGGATAAAAAGGCTTTAAAAGATTGGAAGAT
CCCACTGTTTGGAACTCCGGTAGAGTCGATTGGGTCCAGGAAAAATGCTC
TCACATGCTCATAATACTGCTTGTGGGCTACATTACTGGACGACTTGGTT
GACGGGGGTTTGCTTGAGACTGCTAGCATTCTTCAAGATTACTACTCCAT
AATCCTGAATCATCTACACAATCCTGAAGTCAAGATGCCTGGAGGCATCG
GACAATCACCTTCCAGTTCGAGTTTACAGGGCCACTGAAGAGAGATAAGA
TCATCCATGCTTCCTCCAGTGTATGCTTATTTTGTAGCACAGTTTGAGAG
GTATGCACTCAGCAGAATGGCAAGCAGGCCCTGTCAAGCAGTCTATCGAG
TGGAGAAGGTTTGAAGTGTTATTAGAGCCTATCTTCAGCTTCATAGAGAT
GGCGTTTGAAGTCGCATAGAGATGGCGTTGGAATCAGAGGACTATCTAAT
TCTGCGAGATCCCAGGATTGACTATGTATCTATGCACAACCATATTCTCC
CGTTCGTGAAGGTGTTCGTAACTGCTGAATCTTCCAGTGTTGCTGCTTCT
TTCGGATCCACATTCTGATCGCTCAGGCAAGAGGTGAAGGTAAACACGAG
TTTGTGAAGTATCTTGAGTGTCATCCTAGTGTTCTCTCCAACACGCTTTA
CTATCACTACCCTCTGCCCGGTACCATCCAGCATTCATCACTGGTGAGAA
GTTTGACGGGAATTGGTGTTTGGGCACTGTTATAAACCATAGAAGAACTG
GCCGGTGA
(SEQ ID NO: 41)
ATGGCATTTGTTGTGGAGAAGATTCCCGCCATGGAACACCACCTGGGGCT
AAAGAGGTTCTATTTGCCGCCCATTCGCTGCTCCATCCCCTCCAGCGCCT
GGGATCCCGACCACAAGCTGGTTGCCAAGCTCGCGAACGAGTGGGCATTC
CCATTCATCAATCCCAGCATGAGCGATGCCCAAAAGCTCTCCCTGGAGCG
CATGCGAATCCCGCTCTACATGAGCATGCTCGTGCCGTGCGGATCCACCG
AGAGCGCCGTCCTCTGCGGCAAGTTCGCGTGGTTTGGAACCATGCTGGAT
GATCTCCTCGAGGACGAGTCCCCCGGCGGCGCCCCCCGGGAGGAGTTCCT
GGAGACTTTCCAGGGCATCCTCCATGGGACACACCCACATCGCGATCCAG
TCCATCCATCGCTCGAGTTCTGCGCGGACCTCATTCCGCGCCTGCGATCA
TCCATGGCTCCCCGGGTGTACGCGCACTGGGTCGCGCAGATGGAGGCCTA
CGCTGCCTCCATGGACCGGAGCGTCCTTTCTCTAGCACAATCGGCGTCGA
CGGTCGAGAGCTACTTGGCGCGCAGGAGGCTCGATTGCTTCCTCCTCCCC
TGCTTCCCATTCATCGAGATGTCGCTGGAGATTGCGCTCCCAGACAGCGA
TTTGGAGTCGCGGGACTACCTGGCGCTCCAGAACGCCATCAACGATCACG
TCCTCCTTGTCAACGACGTTATCTCCTTCCCCGCGGAGCTGCGCGCCAAA
AAGCCACTGAGAAGCATCGCGTCCTTGCAGTTGCTCTTGGATCCCAGCGT
CAACACGTTCCAGGACTCGGTGGACAGGACCTGTGCAATGATCCAGGAGA
AGGAACGCGAGGTGACGCATTACTACGACGTTGTGATGAGGAACGCTGTG
GCTTCTGGCAATGCCGAGCTTGTGAGCTACCTTCAGATCCTCAAGATGTG
CGTTCCAAACAACCTCAAGTTCCACTTCATTAGTTCTCGTTATGGAGTGA
ATGATGCCGAGTCTGGTCATGGAATTTGGATTGTTCTGTAG
(SEQ ID NO: 42)
ATGGGCTACGTGGGAGTAAACATGGAAGTTCTCGTGGATTGCCGCAACAC
CGTCTTTGCGAAAGGCCTTACATCACTGGAGGAGCTCTGGTGGTGGTGTT
TTGGACGCCATGGGTTCTTAACTCAATGTACTCTGAAACGGAGATTGATA
TTGTCAAAGGGAACCTGCAGACAATTGAGTATTACCAATAGACCTTTCTC
CTTGTATATTTCTTGGCGGGTGCTACCCAGGCATTATATAGCTTACACAG
CCCTGGAGAAACATAGAAGAAGATCCATCATGGGAGCCTCCTCTATCCTT
AGCATTTTTGAGGGCGCCAAAAGCTTTTACATACCACCGCACAGCTCTTA
TCATGTTGATTTGAATCCTGCTTATGATGCAAAGTTGGATGCTGAAATTG
ACAAATGGTGTATGGATTTTTTGAACCTGCATGATCTCACTGATCACAAG
ACTCAGTTTGCCATTCAGAGCAAGCTTGGAAAACTTGCAGGCTTTGCATA
CCAAGCCATTTCTTCAGAGAGATTGAGTCCCATCGCAAAATTCTTTTGCT
GGTTGTTTCTTGCAGATGATTTTATGGACGATCCTTCTGTCCCTGTCTCT
GACCTGAAGAATGCCACACTTGCATACAAGCTCATCTTCAAAAACGACTA
TGATCAAGCCATAACTCTGGTGGAAAGTAAAGGCCTTCTGCGGCAAATGG
GGATGCTTAACGATGTCTACACTGATCTAAAGGGTTTCATGAATCCTGGA
CATAAGACCCGGTTTTCGAAATCCATGATTGATGTGCTCGACATGTTTGA
GGTGGAATCAAGTTGGCTTCACAAGACACTGGTTCCCAACTTTGAGATTT
ATATGTGGATGAGGGAGGTAACAGCTGGGGTTATCCCTTGCATGGTGGCA
ATGGACTTCCTTAATAATTTTGGGCTGGAAGAGGAAGGAGTGCTAGACGA
TCCCCATATTCAAACGCTGGAAGTGATTGCAAATCGCCATTCGTTCCTAG
CCAATGACATGGTCTCCTTCAAAAAGGAATGGGCTTGTGAACAGTACCTC
AATTCTGTGGCACTGGTTGGTTACAGTAGCAACTGTGGCTTAAACGAGGC
AATGGAGAAAGTTGCACAAATGGTTCAGGATTTGGAGAAAGAGTTTGCTG
ACATCAAACAAAAGGTTCTCTCAAACAAGGACTTGAACAAGGGAAATGTC
ATGGGGTATGTGCAAAGCTTGGAGTATTTCATGGCTGCAAATATAGAGTT
TAGCTGGATCTCTGCGAGATATCATGGGGTGGGATGGGTTTCACCAGCTG
AGAAATATGGTACCTTTGAGTTCTAG
(SEQ ID NO: 43)
ATGAGGAGGGGCAGCGCCTACACCAAGCAAGAGCTCCTCGCGCATCACAT
GGGCTACATGGGAGTAAACATGGAAGTTCTCATGGATTGCCGCAACACCC
TCTTTGCGAAAGACCTTACATCACTGGAGGAGCTCTGGCGGTGGTGTTTT
GGACGCTGTGTTTGGTGTACGCCACCCAGGAACATGCGGTGGACGAGACT
TTGGTGCTTCTTTGAGGAGTTTACATCGTCCGATATCAATCTCCATTTCA
GAGTACATTGGGTTGAGGGTCAAGCAGAATTAGACATTGGAAGTAGAAAG
GTGGATGCTGAAATTGACAAATGGTGTATGGATTTTTTGAACTTGCACGA
TCTCACTGATCACAAGACTCAGTTTGCCATTCAGAGCAAGCTGGGAAAAC
TTGCAGGCTTGGCATACCAGGCCATTTCTTCAGAGAGACTCCGTCCCATG
GCAAAATTCTTGTGCTGGTTGTTTCTTGCAGATGATTTTATGGAAGATTC
TTCTGTCCCTGTCTCTGACCTGAAGAATGCCACACTTGCATACAAGCTCA
TCTTCAAAAACAACTATGATCAAGCCATAACTCTGGTGGAAAGTAAAGAC
CTTCTGCGGCAAATGGGGATGCTTAACGATGTCTACACTGATCTAAAGGG
TTTCATGAAGCCTGGACATAAGACCCGGTTTTCGAATTCCATGATTGATG
TGCTCGACATGTTTGAGGTGGAATCAACTTGGCTTCACAAGAAACTGGTT
CCCAACTTTGAGATTTATATGTGGATGAGGGATGTAACGGCTGGGGTTAT
CCCTTGCATGGTGGCAATAGACTTCCTTAATAATTTTGGGCTGGAAGATG
AAGTGCTAGAGCATCCCAACATTCAAAGGCTGGAAGTGATTGCAAATCGC
CACACGTACCTAGCCAATGACATGCTCTCCTTCAAAAAGGAATGGGCTTG
CGACATGTACCTCAATTCTGTGGCACTGGTTGGTTACAGTAGCAACTGTG
GCTTAAATGAGGCAATGGAAAAAGTTGCAGAAATGGTTCAGGATTTGGAG
AAAGAGTTTGCTGACACCAAACAAAAGGTTCTCTCAAACAAAGACTTGAA
CAAGGGAAATGTCATGGGGTATGTGCAAGGCTTGGAGTATTTCATGGCTG
GAAATCTAGAGTTTGCCTGGCTCTCTGCGAGATATCATGGGGTGGGATGG
GTTTCACCAGCCGAGAAATATGGTACCTTTGAGTTCTAG
(SEQ ID NO: 44)
ATGGGACCCTCCTCTATCCTTAGCATTTTTGAGGGCGCCAAAAGCTTTTA
CATACCACCGCACAGCTCTTATCATGTTGATTTGAATCCTGCAAAATTGG
ATGCTGAAATTGACAAATGGTGTATGGATTTTTTGAACCTGCACGATCTC
ACTGATCACAAGACTCAGTTTGCCATTCAGAGCAAGCTGGGAAAACTTGC
AGGCTTGGCATACCAGGCCATTTCTTCAGAGAGACTGCGTCCCATGGCAA
AATTCTTGTGCTGGTTGTTTCTTGCAGATGATTTTATGGACAATCCCTCT
GTCCCTGTCTCTGACCTGAAGAATGCCACACTTGCATACAAGCTCATCTT
CAAAAACGACTATGATCAAGCCATAACGCTGGTGGAAAGTAAAGACCTTC
TGCGGCAAATGGGGATGCTTAACGATGTCTACACTGATCTAAAGGGTTTC
ATGAATCCTGGACATAGGACCCGGTTTTCGAAATCCATGATTGATGTGCT
CGACATGTTTGAGGTGGAATCAAGTTGGCTTCACAAGAAACTGGTTCCCA
ACTTTGAGATTTATAACGTAACGGCTGGGGTTATCCCTTGCATGGTGGCA
ATAGACTTCCTTAATAATTTTGGGCTGGAAGATGATGTGCTAGACCATCC
CAACATTCAAAGGCTGGAAGTGATTGCAAATCGCCACACGTACCTAGCCA
ATGACATGGTCTCCTTCAAAAAGGAATGGGCTTGCGACATGTACCTCAAT
TCTGTGGCACTGGTTGGTTACAGTAGCAACTGTGGCTTACACGAGGCAAT
GGAGAAAGTTGCACAAATGGTTCAGGATTTGGAGAAAGAGTTTGCTGACA
TCAAACAAAAGGTTCTGTCAAACAAGGACTTGAACAAGGGAAATGTCATG
GGGTATGTGCAAGGCTTGGAGTATTTTATGGCTGGAAATATAGGCTCTCT
GCGAGATATCATGGGGTGGGATGGGTTTCACCAGCTGAGAAATATGGTAC
CTTGGAGTTCTAGTTTGCTTCTACTTGCATTAGAAGCTGGTGCTTAA
(SEQ ID NO: 45)
ATGGCCGCGCCTTCTATCTATCGTCCCCAAATTCTGGAGCAGCTCCTCGC
CTGCAAGAGCATCTACTTGCCTCAAATCCGCTGCTCGCTGCCATTGCAGT
GCCACCCAGACTACGCCTCCGTCTCCAGACAGGCGAACGATTGGGCCTTT
CGCTTCCTCAAGATCAATGCCACCAATGCCGCTGCAGAGAAGAAATGCTT
CACCCAGTGGAGGACGCCACTCTACGGCACCTTCGTTGTGCCTTGGGGCG
ACTCCAGGCACGCTCTAGCGGCCGCCAAGTACACCTGGCTCATCACCATT
CTCGACGATGCTGTCGACGAGGAGCCTTCGCAGCGGAACGAGATCCTGGA
AGCTTACATGAGCCTTGCCTCCGGAAATTTGTTGGCTGCTACTCGGACGA
AGAGGAGTTCACGAAAGAGTCTTAACGCTGTGCATCGCCGGGAGGACTTC
GTTGTCAAGCCGATGCTTAACTTCACGCAGATGTGCCTCGGAGTGAAGCT
GAGAGACAAGGATCTGGAAAGCGAGGAGTACCTCCGGGCGATAGATGCCA
TGTTTGATCACATCTGGCTGGTGAACGACATCTTTTCATTCCCAAAGGAG
CTGAGGAAGAAAACTTTCAAGAACATAATTTTTCTCTTGCTCTTCACGGA
CCACACCGTTCGCTCTGTTCAGCAGGCAGTCGATAAGGCGAATGCCATGG
TTCAGGAAAAAGAACAAGAATTCATGTATTACCACGAGATCCTGACGAGG
AAAGCGATGGAATCTGGCAACCACGACTTTCTGGCGTACCTTAGAGCGAT
TCCGGCATTCATCCCTGGAAACCTACGTTGGCACTACCTCACAGCTCGGT
ACCACGGTGTTGATAATCCATTTGTAACAGGAGAGCCATTCAGTGGGACT
TGGTTGTTTCATGATACGCAGACTATCATACTCCCCGAGTACAAACCAAC
TCATCCCCATCTGCAAGTCTGA
(SEQ ID NO: 46)
ATGGCCGCGCCTTCTATCTATCGTCCCCAAATTCTTGAGCAGCTCCTCGC
CTGCAAGAGCATCTACTTGCCTCAAATTCGCTGCTCGCTGCCATTGCAGT
GCCACCCAGACTACGCCTCCGTCTCCAGACAGGCGAACGATTGGGCCTTT
CGCTTCCTCAAGATCAATGCCACCAATGCCGCTGCCGATAAGAAATACTT
CACCCAGTGGAGGATGCCACTCTACGGCACCTTTGTTGTGCCTTGGGGCA
ACTCCAGGCACGCTCTAGCGGCCGCCAAGTACACCTGGCTCATCACCATT
CTCGACGATGCTGTCGACGAGGAGCCTTCGCAGCGGGACGAGATCCTGGA
AGCTTACATGAGCCTTGCCTCCGGTCAAAGATCCATCGCCCAAGTTCCCA
ACAAACCCGTGCTCGTCGCCCAAGCCGAGCTCGTCCCGGATCTGCGGAAG
CTCATGTCGCCGCTCCTCTTCCAGCGGCTGCTCGTCTCGTACAGGAAATT
TGTTGGCTGCTACTCGGCCAAAGTCGACGAGGAGGAGTTCACGAAAGAGT
CTTACGCTGTGCATCGCCGGGAGGACTACGTTGTCAAGCCGATGCTTAAC
TTCACGCAGATGTGCCTGGGAGTCGAGCTGAGAGACAAGGATCTGGAAAG
CGAGGAGTACCTGCGGGCGATAGATGCCATGTTTGATCATATGTGGCTGG
TGAACGACATCTTTTCATTCCCAAAGGAGCTGAGGAAGAAAACTTTCAAG
AACATAATTTTTCTCTTGCTCTTCACGGACCACACCGTTCGCTCTGTTCA
ACAGGCAGTTGATAAGGCGAACGCCATGATTCAGGAAAAAGAACAAGAAT
TCATGTATTACCACGAGATCCTGACGAGGAAAGCGATGGAATCTGGCAAC
CACGACTTTCTGGCGTACCTTAGAGCGATTCCGGCTTTCATCCCTGGAAA
TCTACGTTGGCACTACCTCGCAGCTCGGTACCACGGTGTTGATAATCCAT
TTGTAACAGGAGAGCCATTCAGTGGGACTTGGTTGTTTCATGATACACAG
ACTATCATACTCCCCGAGTACAAACCAACTCATCCCCATCTGCAAGTTTA
A
(SEQ ID NO: 47)
ATGGGGATGCTTAACGATGTCTACACTGATCTAAAGGGTTTCATGAATCC
TGGACATAAGACCCAGTTTTCGAATTCCATGATTGATGTGCTCGACATGT
TTGAGGTGGAATCAAGTTGGCTTCACAAGAAACTGGTTCCCAACTTTGAG
ATTTATATGTGGATGAGGGAGGAATGGGCTTGTGAACAGTACCTCAACTC
TGTGGCACTGGTTGGTTACAGTAGCAACTGTGGCTTAAACAAGGCAATGG
AGAAAGTTGCAGAAATGGTTCAGGATTTGGAGAAAGAGTTTGCTGACATC
AAACAAAAGGTTCTGTCAAACAAGGACTTGAACAAGGGAAATGTCATGGG
GTATGTGCAAAGCTTGGAGTATTTCATGGCTGCAAATATAGAGTTTAGCT
GGATCTCTGCGAGATATCATGGGGTGGGATGGGTTTCACCAGCTGAGAAA
TATGGTACCTTGGAGTTCTAG

SEQ ID NOS: 48-89 are exemplary amino acid sequences of Selaginella moellendorffii terpene synthases from the SmMTPSL genes.

(SEQ ID NO: 48)
MAILSIVSIFAAEKSYSIPPASNKLLASPALNPLYDAKADAEINVWCDEF
LKLQPGSEKSVFIRESRLGLLAAYAYPSISYEKIVPVAKFIAWLFLADDI
LDNPEISSSDMRNVATAYKMVFKGRFDEAALLVKNQELLRQVKMLSEVLK
ELSLHLVDKSGRFMNSMTKVLDMFEIESNWLHKQIVPNLDTYMWLREITS
GVAPCFAMLDGLLQLGLEERGVLDHPLIRKVEEIGTHHIALHNDLISFRK
EWAKGNYLNAVPILASIHKCGLNEAIAMLASMVEDLEKEFIGTKQEIISS
GLARKQGVMDYVNGVEVWMATNAEWGWLSARYHGIGWIPPPEKSGTFQL
(SEQ ID NO: 49)
MALALDKIYAIEKLLGLKNFHLPKIPCSIPSVPCHPDSIYASNKAHEWAY
KFMDPKMTAADRKALEDWKIPMFATLVVPFGSKRNAVICSKYSMFALLVD
DSVDEGFVESTILQDYYSTILNHLHNPNFKIQASDDHLPLRVYRATEELV
TEIRSSMLPPVYAHFVAQFERYALSRMASRPKFQSVKQYIEWRRFDVFLE
PIFSFIEMALEVAVPDTELESEDYLILRDAGIDYISMYNDVLSFAKEFAC
NKLLNLPVLLLLSDPEVESFQNAVDKSCKMIVDKEQEFVYYHNILITQAR
GEGKHAFVKYLECLPTVLSNTLYYHYSSARYHPAFITGEKFDANWCLDTV
INHRRTGR
(SEQ ID NO: 50)
MAAPSIYRPQILEQLLACKSIYLPQIRCSLPLQCHPDYASVSKQANDWAF
RFLKINATNAAADKKYFTQWRMPLYGTFVVPWGDSRHALAAAKYTWLITI
LDDAVDEEPSQRDEILEAYMSLASGQRSIAQVPNKPVLVAQAELIPDLQK
LMSPLLFQRLLVSYRKFVGCYSAKVDEEEFTKESYAVHRREDYVVKPMLN
FTQMCLGVELRDKDLESEEYLRAIDAMFDHMWLVNDIFSFPKELRKKTFK
NIIFLLLFTDHTVRSVQQAVDKANAMIQEKEQEFMYYHEILTRKAMESGN
HDFLAYLRAIPAFIPGNLRWHYLTARYHGVDNPFVTGEPFSGTWLFHDTQ
TIILPEYKPTHPHLQV
(SEQ ID NO: 51)
MAAPSIYRPQILEQLLACKSIYLPQIRCSLPLQCHPDYASVSKQANDWAF
RFLKINATNAAADKKYFTQWRMPLYGTFVVPWGDSRHALAAAKYTWLITI
LDDAVDEEPSQRDEILEAYMSLASGQRSIAQVPNKPVLVAQAELIPDLQK
LMSPLLFQRLLVSYRKFVGCYSAKVDEEEFTKESYAVHRREDYVVKPMLN
FTQMCLGVELRDKDLESEEYLRAIDAMFDHMWLVNDIFSFPKELRKKTFK
NIIFLLLFTDHTVRSVQQAVDKANAMIQEKEQEFMYYHEILTRKAMESGN
HDFLAYLRAIPAFIPGNLRWHYLAARYHGVDNPFVTGEPSSGTWLFHDTQ
TIILPEYKPTHPHLQV
(SEQ ID NO: 52)
MAPYDFVPNVQCSFPVKCHPLYSFIRPGLEDWAATLEPGHGEGNPKGLGA
DLGGAKRLVDSYLGIIHAPEPVADMEFPRFCDMWNDLRADMPLKQYQRFA
NRVSELLKASVNQVRLRNLKTVMGLEELLAHRRMLVGVFVMETLMEYGMG
FELQDDAISNQDLQEAESLVADHCNWRYSVQYRPCGNSDSKGFSFEYAAD
KVQKLVQSIEHRFKKLCENIRRSSCYNGAMEAYLEGLSHIISGNLEWHRQ
TGRYKLVS
(SEQ ID NO: 53)
MAASVNGVLPELSTLSKFELRPLPCAFPFECHPNHASLTREVDEWAIRSL
QARGSMPKRQMIIESKISAAACMTIPRGRDDRKMVLAGKHLWALFLLDDA
LESCRSQEAARVLARRAMEVARGDQLEGMIQEERELEEAKGVARKFAIQE
EEGDRYNDQSRGILANIAIQEDPGLIDLATRGMATKIAITEEDQGRDSRW
ALGLFREVVAELRRSMPLPMFDRYLRYLDRYLEAVIQEVGYQIAGHIPRE
DEYRELRRGTSFTEGTSAIFGELCMGLELHESVTSSRDFIEFVALVADHI
ALTNDVLSFRKDFYAGVAHNWLVVLLRHSHRGTGFQSALDSVYGMIRDSE
CRILGLQSRIEAQALKSGDGHLLSFAQAFPLCLAGNRRWSSITARYHGIG
NPLITGVEFHGTWLLHPDVTIVI
(SEQ ID NO: 54)
MALAVEKIPAMEHLLGLKRFYLRPIRCSIPSSAWHPDHKLVAKLANEWAF
PFINPSMSDAQKLSLERMRIPLYMSMLVPCGSTESAVLCGKFAWFGTMLD
DLLEDESPGGAPREEFLETFQGILHGTHPHRDPVHPSLEFCADLIPRLRS
SMAPRVYAHWVAQMEAYAASMDRSVLSLAQSASTVESYLARRRLDCFLLP
CFPFIEMSLEIALPDSDLESRDYLALQNAINDHVLLVNDVISFPAELRAK
KPLRSIASLQLLLDPSVNTFQDSVDRTCAMIQEKEREVTHYYDVVMRNAV
ASGNAELVSYLQILKMCVPNNLKVHFISSRYGVNDGESGHGIWIVL
(SEQ ID NO: 55)
MSSFRSLMDPPLFARYMICLKTFLDSLVEEASLRSAKSIPSLEKYQLLRR
GTVFVEGAGDFVAFVNAVADHVLFSFRHEMKIKCFHNYLCVIFCHSPNNA
SFQEAVDKVCKMIQETEAKILQLQKKLMKMGEETGNKDLVDYATWYPCFT
SGHLRWPWA
(SEQ ID NO: 56)
MSSFRSLMDPPLFARYMICLKTFLDSLVEEASLRSAKSIPSLEKYQLLRR
GTVFVEGAGGIMCEFCMDLKLDKVADHVLFSFRHEMKIKCFHNYLCVIFC
HSPNNASFQEAVDKVCKMIQETEAKILQLQKKLMKMGEETGNKDLVDYAT
WYPCFTSGHLRWVYVTGRYHGLDNPLLNGEPFHGTWFLHPEATFILPFGS
KCGFINTM
(SEQ ID NO: 57)
MALPSLLSTKLKPLELLSGVTHYDLPPIPCSLPVKCHPQFAKFSRIADTW
AIDAMQLQNDPCGKLKAVQSRAPLLYCFLVPFGIGEEEMIAGCKYSWSTS
FVDDPFDEETDLKRAKEWKKVVLRAANGTPSAEDLMIRTIKAYSEIMMHL
QQMMAAPVFSRFMRAHYAWADHCVELVRRRQHKDPPTVATYLADRCENLL
VEPIFILAEVCMKLQIDPEFLSLPEFKKIWTTMLEHAAIVNDVLSIRVDI
LNGHYYTYPGLVFQQHPEIQTFQEAVDYSVGMIQTKERKFIKLHEMLTDK
ARQCGFKNKSDLLKYVEALPNFIAGNLYWHYLSARYFGVNNPFLTGEPVQ
GTILIHPRNTVVLPPYQRNKHPFLIDVDNLELGA
(SEQ ID NO: 58)
MRSFSSFHISPMKCKPALRVHPLCDKLQMEMDRWCVDFASPESSDEEMRS
FIAQKLPFLSCMLFPTALNSRIPWLIKFVCWFTLFDSLVDDVKSLGANAR
DASAFVGKYLETIHGAKGAMAPVGGSLLSCFASLWQHFREDMPPRQYSRL
VRHVLGLFQQSASQSRLRQEGAVLTASEFVAGKRMFSSGATLVLLMEYGL
GVELDEEVLEQPAIRDIATTAIDHLICVNDILPFRVEYLSGDFSNLLSSI
CMSQGVGLQEAADQTLELMEDCNRRFVELHDLITRSSYFSTAVEGYIDGL
GYMMSGNLEWSWLTARYHGVDWVAPNLKMRQGVMYLEEPPRFEPTMPLEA
YISSSDSC
(SEQ ID NO: 59)
MEAIVSSSKIHAVEHLLSLKSYSLPQILLAHPVKCHPDYTSICKESDEWI
FSYLGVTSPEHKKRLAQWRVPIFAAFLTPPSSPKRRTLLGGKFTWLITAL
DDQLDESKISQAGRSCQYRDAILSIFSGRSDYPAILPAEVPLLRACEELM
PEIRSFMLPPTLNRFLAYTKQWSQTFDVAYESTQVFKELRRDNVWITAYF
PMIEMFLGLGLGDDVAGSKDFLAAQDAISDHAWMVNDLFSFAKEFRDEKK
LSNILSVSLLMDSCVHTIQDAIDLLCTELQAKEEEFLYYHGILVKRAQAG
NNQDLLRYLEAILAVIPGNLHFHYITARYHGYNNPCVNGEAWHGKVILQP
NTLGPPPKPHPYLYDI
(SEQ ID NO: 60)
MAVSSIVSIFAAEKRYSIPPVCKLLASPVLNPLYDAKAESQINAWCAEFL
KLQPGSEKAVFVQESRLGLLAAYVYPTIPYEKIVPVGKFFAWYFLADDIL
DSPEISSSDMRNVTTAYKMVLKGKFDEATLPVKNPELLRQMKMLSEVLEE
LFLHIVDESGRFVDALTKVLDMFEIESSWLRKQIIPNLDTYLWLREITSG
VAPCFALIDGLLQLRLEERGVLDHPLIRKVEEIGTHHIALHNDMISFRKE
WAKGYYLNAVPILASNCKCGLNEAIGKVASMVEDVEKDFAQTKHEIVSSG
LAMKQGVMDYVNGIEVWMAGNVEWAWTSARYHGIGWIPPPEKSATFQL
(SEQ ID NO: 61)
MARTLFNDMLKQAALPDIVTFSTLVEGYCNAGLVDDAERLLEEIIASDCS
PDVYTYTSLVDSFCKVKRMVEAHRVLKRMAKRGCQPNVVTYTALIDAFCR
AGKPTVAYKLLEEMVGINNDVQPNVQELASVGLGTWKRLARCSRDWSATR
TARRICSHTGGLCQGKELSKAMEVLEEMTLSRKGRPNAEAYEAVIQELAR
EGRHEEANALADELLGNKGHLLSVFKIHLGSIHCEHFRSGEKLFHSTKSR
LGLLAAYVYPTIPYEKIVPVAKFIAWFFLADDILDSPEISSSDIRYVATA
YKMVFKGRFDEATLPVKNPELLRQMKMLAEVLEELSLHIVDESGRFVDAM
TKVLDMFEIESSWLRKQIIPNLDTYLWLREITSGVAPCFALIDGLLQLRL
EERGVLDHPLIRKVEEIGTHHIALHNDLILLRKEYFLASDYDVDLPSSEA
SSTLFFLLQMATFMKYFLEDLCSHFAARCRIIPYKNVSSLWMDQSGAVLQ
KKLLKLEFTTLFEYLQRLSPTSTSPGTPW
(SEQ ID NO: 62)
MAVSSIASIFAAEKSYSIPPVCQLLVSPVLNPLYDAKAESQIDAWCAEFL
KLQPGSEKAVFVQESRLGLLAAYVYPTIPYEKIVPVGKFFASFFLADDIL
DSPEISSSDMRNVATAYKMVLKGRFDEATLPVKNPELLRQMKMLSEVLEE
LSLHVVDESGRFVDAMTRVLDMFEIESSWLRKQIIPNLDTYLWLREITSG
VAPCFALIDGLLQLRLEERGVLDHPLIRKVEEIGTHHIALHNDLMSLRKE
WATGNYLNAVPILASNRKCGLNEAIGKVASMLKDLEKDFARTKHEIISSG
LAMKQGVMDYVNGIEVWMAGNVEWGWTSARYHGIGWIPPPEKSGTFQL
(SEQ ID NO: 63)
MAVSSIASIFAAEKSYSIPPVCQLLVSPVLNPLYDAKAESQIDAWCAEFL
KLQPGSEKPVFVQESRLGLLAAYVYPTIDCSDDILDSPEISSSDMTNVAT
AYKMVLKGRFDEAMLPVKNPDLLRQMKMLSEVLEELSLHVVDESGRFVDA
MTRVLDMFEIESSWLRKQIIPNLDTYLWLREITSGVAPCFALIDGLLQLR
LEERGVLDHPLIRKVEEIGTHHIALHNDLMSLRKERATGNYLNAVPILAS
NRKCGLNEAIGKVASMLEDLEKDFARTKHEIISSGLAMKQGVMDYVNGIE
CFRNSYLSSVFDLNKQIEMHGRCGNIKHAAQIFHASCCDFPSWEASSTLF
FLLQMPFCRSLPDNPWAVLLKKLLKLEFTTLFEYLQLTSTSPGTPW
(SEQ ID NO: 64)
MEATLISKFSTVTHFELPQLPNNIPFAYHPQSATISAQIDEWMLRKMKIT
DQSARKKMIHSKMGLYACMMHPNAEREKLVLAGKNLWALLLMDDLLESSS
KEEMPRLNTTISSLGSGNSGDGAIRNPVLLLYKEVLGELRAAMEPPLLDR
YLHCLAASLEGVRKQVHHRTKKSVPGPEEYKFTRRANGFMDILGGIMTEF
CMGIRLNQAQIQSPTFRELLNSVSDYVILVNDLLSFRKEFYGGDYHHNWI
SVLSYHGPSGISFQDVIDQLCEMIQAEEHSILALQKKIADEEGCDSELTK
FASELAMVASGSLVWSYLSGRYHGYDNPLITGEIFSGTWLLHPVATVVLP
SIKARDTLLGLKVPVPLP
(SEQ ID NO: 65)
MEDVLVSRILGVTHFELPLLPNNIAFYCHPEFQSISLQIDEWFLDKMRIA
DETSKKKVLESRIGLYACMMHPHAEREKIVLAGKHLWAVFLLDDLLESSG
TQEMPKLNATISDLASGNSNEDVTNPVLVLYREVMEEIRAGMEPPLLDRY
VECLGASLEAVKDQVHHRAEKSIPGVEAYKLARRATGFMEAVGGIMTEFC
MGIRLNESQIQSPVFRELLNSVSDHVVLVNDLLSFRKEFYEGACHHNWIS
VLLQHSPSGTRFQDVIDQLCEMIQEEELSILALQRKISSKENSDSELMKF
AREFPMVASGSLVWSYVTGRYHGYGNPLLTGEIFSGTWLLHPMATVVLPK
STVFSLNHLVYSHVIL
(SEQ ID NO: 66)
MEDILVSRISGVTHFELPLLPNNIAFYCHPEFQSISLQIDEWFLAKMRIT
DETSKKKVLESRIGLYACMMHPHAEREKIVLAGKHLWAVFLLDDLLESSG
TQEMPKLNATIFNLASGNSNEDVTNPVLVLYREVMEEIRAGMEPPLLDRY
VECLGASLEAVKDQVHHRVEKSIPGVEEYKLARRATGFMEAVGGIMTEFC
MGIRLNESQIQSPVFRELLNSVSDHVVLVNDLLSFRKEFYEGACHHNWIS
VLLQHSPRGTRFQDAIDQLCEMIQEKELSILALQRKISSKEHSDSELMKF
AREFPMVASGSLVWSYVTGRYHGYGNPLLTGEIFSGTWLLHPMATVNGYQ
TILVYSLINNTEIKSIISTIYTVSQIASSG
(SEQ ID NO: 67)
MKDLFRISGVTHFELPLLPNNIPFACHPEFQSISLKIDKWFLGKMRIADE
TSKKKVLESRIGLYACMMHPHAKREKLVLAGKHLWAVFLLDDLLESSSKH
EMPQLNLTISNLANGNSDEDYTNPLLALYREVMEEIRAAMEPPLLDRYVQ
CVGASLEAVKDQVHRRAEKSIPGVEEYKLARRATGFMEAVGGIMTEFCIG
IRLSQAQIQSPIFRELLNSVSDHVILVNDLLSFRKEFYGGDYHHNWISVL
LHHSPRGTSFQDVVDRLCEMIQAEELSILALRKKIADEEGSDSELTKFAR
EFPMVASGSLVWSYVTGRYHGYGNPLLTGEIFSGTWLLHPMATVVLPSKF
RMDTMRFSLAPKKRDSFP
(SEQ ID NO: 68)
MEATLISKFSTVTHFELPQLPNNIPFAYHPQSATISPQIDEWMLRKMKIT
DQSVRKKMIHSKIGLYACMMYPNAEREKLVLAGKNLWALLLIDDLLESSS
KEEMPRLNTTITNLGSGNSRDGAIRNPVLLLYKEVLGELRAAMEPPLLDR
YLHCLAASLEGVRKQVHHRTRKSVPGPEEYKLTRRANGFMDILGGIMTEF
CMGIRLNQAQIQSPTFRELLNSVSDYVILVNDLLSFRKEFYGGDYHDNWI
SVLSYHGPRGISFQDVIDQLCEMIQAEEHSILALQKKIADEEGCDSELTK
FASELAMVASGSLVWSYLSGRYHGYDNPLITGEIFSGTWLLHPVATVVFP
SIKARP
(SEQ ID NO: 69)
MFEDVMLSIQSLMDPPLFARYMICLRNYLDALVEDSSLRFAKSIPSLTKH
QLLRKQLEALYRDKHYSYLCVIFCHDNASFQGTVDKACEMIQETEGEILQ
LQKKLMKLGEETGNKDLVEYARYPCVASRNLRWSYVTRTSSREPFHATWF
LLPEVTLIVPFGSKCGDHPFAITENHLV
(SEQ ID NO: 70)
MEDVLAERLSRVSKFDLPSIPCSIPLESHPEFSRISEVTDAWAIRMLGIT
DPYERQKAIQARFGLLTALATPRGESSKLEVASKHFWTFFVLDDIAETDF
GEEEGQKAADILLEVAEGSYVFSEKEKQKNPSYAMFEEVMSSFRSLMDPP
LFARYMTCLKNFLDSVVEEASLRFAKSIPSLEKYQLLRRETVFVEASGGI
MCEFCMDLKLDKGVVESPEFVAFVKAVVDHAALVNDLLSFRHEMKIKCFH
NYLCVIFFHSPDNASFQETVDKVCKMIQETEAEILQLQKKVMKMGVETGN
KDLVEYATWYPCFASGHLRWSYVTGRYHGLDNPLLNGEPFHGTWFLHPEV
TLMLPFGAKCGDHPWIARS
(SEQ ID NO: 71)
MEDVLAEKLSRVCKFDLPFIPCSIPFECHPDFTRISKDTDAWALRMLSIT
DPYERKKALQGRHSLYSPMIIPRGESSKAELSSKHTWTMFVLDDIAENFS
EQEGKKAIDILLEVAEGSYVLSEKEKEKHPSHAMFEEVMSSFRSLMDPPL
FARYMNCLRNYLDSVVEEASLRIAKSIPSLEKYRLLRRETSFMEADGGIM
CEFCMDLKLHKSVVESPDFVAFVKAVIDHVVLVNDLLSFRHELKIKCFHN
YLCVIFCHSPDNTSFQETVDKVCEMIQEAEAEILQLQQKLIKLGEETGDK
DLVEYATWYPCVASGNLRWSYVTGRYHGLDNPLLNGEPFQGTWFLHPEAT
LILPLGSKCGNHPFIMI
(SEQ ID NO: 72)
MEDVLAEKLSRVCKFDLPFIPCSIPFECHPDFTRISKDTDAWALRMLSIT
DPYERKKALQGRHSLYSPMIIPRGESSKAELSSKHTWTMFVLDDIAENFS
EQEGKKAIDILLEVAEGSYVLSEKEKEKHPSHAMFEEVMSSFRSLMDPPL
FARYMNCLRNYLDSVVEEASLRIAKSIPSLEKYRLLRRETSFMEADGGIM
CEFCMDLKLHKSVVESPDFVAFVKAVIDHVVLVNDLLSFRHELKIKCFHN
YLCVIFCHSPDNTSFQETVDKVCEMIQEAEAEILQLQQKLIKLGEETGDK
DLVEYATWYPCVASGNLRWSYVTGRYHGLDNPLLNGEPFQGTWFLHPEAT
LILPLGSKCGNHPFITI
(SEQ ID NO: 73)
MEFLLGKIVPRFELPLLPNNIPCACHPDSSSLSQELDEWFISKLGITDES
AQKKIVQSRIMIFACLMHPNGERDRVLLAGKHLWVCFLVDDILESSTREA
YGSLKSIVWSIATTGIYKASNEEHDHCLVLLLYQEVLAELRKKMPSSLFT
RYCKILSSYLDGVEEEVKHQVKNTIPSSEEYRLLRRRTGFMEVMACIMTE
FCVGIKLEESVVNLGEIRKLVKVMDDHIVMVNDLLSLRKEYYSSTICHNW
VFVLLADGCGTFQESVDHVCEMIKQEEGSILDLQQKLIIKAKVDKNPELL
KFACNVPMAVAGHLKWSFITARYHGCDNALLNGELFHGTWLMDPNQTIIQ
KNI
(SEQ ID NO: 74)
MAVSSIASIFAAEKSYSIPPVCQLLVSPVLNPLYDAKAESQIDAWCAEFL
KLQPGSEKAVFVQESRLGLLAAYVYPTIPYEKIVPVGKFFASFFLADDIL
DSPEISSSDMRNVAIAYKMVLKGRYDEATLPVKNPELLRQMKMLSEVLEE
LSLHVVDESGRFVDAMTRVLDMFEIESSWLRKQIIPNLDTYLWLREITSG
VAPCFALIDGLLQLRLEERGVLDHPLIRKVEEIGTHHIALHNDLMSLRKE
WASGNYLNAVPILASNRKCGLNEAIGKVASMVEDLEKDFAQTKHEIISSG
LAMKQGVMDYVNGIEVWMAGNVEWGWTTARYHGIGWIPPPEKSGTFQL
(SEQ ID NO: 75)
MECLMAKLVPRLELPLLPNNIPSACHWDSSSLSQELDQWLISKLGITDES
AKRKIVQSRVMLLACLMHPNGERDRVLLAGKHLWVYFLVDDILESSSREG
YGALKSIVWSIATTGIYKASEEHDHHDLVLLLLVEVMVELRKEMPTSLFA
RYCKILSIYLDSVQEEVKHQINNTIPSSEEYRLLRRRTGFMEVMACIMTE
FCVGINLEELVVNLGEIRELVKIMDDHIVTVNDLLSLRKEYYNGTIYHNW
VIVLLAHDCATFQKSVDRVCEMIKQEEDSILDLQKKLIIKAKVDKNPELL
KFAFNVPMAVAGHLKWAFITARYHGCDNALLDGELFHGTWIMDPNQTVIV
KNM
(SEQ ID NO: 76)
MGMLNDVYTDLKGFMKPGHKTRFSNSMIDVLDMFEVESSWLHKKLVPNFE
IYMWMREVTAGVIPCMVAIDFLNNFGLEEEGMLDDLHIQTLEVIANRHSF
LANDMVSFKKEWACEQYLNSVALVGYSSNCGLNEAMEKVAEMVQDLEKEF
ADIKQKVLSNKDLNKGNVMGYVQGLEYFMAGNIEFSWLSARYHGVGWVSP
AEKYGTLEF
(SEQ ID NO: 77)
MASPCLQKLPAVEHLFALTRFELPEIPCSLSFQRHPEYMSITKEANEWAF
KCMRRDFSPEEKKCLVQWKVPMFTCLSTPHAPKANMVASAKFAWLTAFLD
DPFDDNEVAGGALATSYLDTVLSLCYGTASLAEIPDILAYRACHDLMKDL
RSLLKPKLFKRTVSTVEGWARSISSDDLTQDYELYRRKNVFILPLIYAMG
ASFDDEDVESLDYIRAQNAMLDHMWMVNDVFSFPKEFYKKKFNNLPAVLL
LTDPSVQTFQDAVNTTCRMIQDKEDEFIYYRDILATNASRNGKKDFLKFL
DVLSCAIPANLVFHYASSRYHGMDNPLLGGPTFSGTWILDPKRTIILSDP
KRWNVVASSNKLNQIQNLSNLI
(SEQ ID NO: 78)
MGALFDDEDVESLDYISAQNAMLDHMWMVNDVFSFLKEFYKNKFNNLPAV
LLTDQSVQTFQDAVNTTWRMIQDKEDEFIFYRDILAANASRNGKKDFLKF
LDVLSCAIPANLVYASSHYHGVDNLLSGGTFRGTWILDPKRTIIVSDPKS
CNVVATTDEVKINVSYAWLFVILILAN
(SEQ ID NO: 79)
MGSLCLQKLSAVERLFALESFELPEVPCSLSFHRHPEYKSITREANEWAF
KCTRRDLSPEEKKSLLQWKVPMVTCLSTAHAPKENMVASAKFAWAIAFLD
DPIDDNEVAATSYLDTVLSLCNGTASLAEVPDIVAYRACHDLMKDLRSLL
QPELFKRTVSTVEGWARSISSDDLKQDYKLYRRNNIFILPLFYTLIGASF
EDEDVESPDFVSAQNAMLDHIWMVNDIFSFRNEFYKKKLNNLPAVLLLTD
PSVQTFQEAVNATCRMIQDKEEEFIYYRNILAANASRNGKDFLKFLDVLS
CAIPANLAFHYASSRYHGMDNPLLAGGTFHGTWILDPKRTIIVSDPNRSN
GAASNKLNHIQDLSKLI
(SEQ ID NO: 8)
MAVYKQGSGFKTEASVILGVTHFELPLLPNNIAFYCHPEFQSISLQIDEW
FLDKMRIADETSKKKVLESRIGLYACMMHPHAEREKIVLAGKHLWAVFLL
DDLLESSGTQEMPKLNATISDLASGNSNEDVTNPVLVLYREVMEEIRAGM
EPPLLDRYVECLGASLEAVKDQVHHRAEKSIPGVEAYKLARRATGFMEAV
GGIMTEFCMGIRLNESQIQSPVFRELLNSVSDHVVLVNDLLSFRKEFYEG
ACHHNWISVLLQHSPSGTRFQDVIDQLCEMIQEEELSILALQRKISSKEN
SDSELMKFAREFPMVASGSLVWSYVTGRYHGYGNPLLTGEIFSGTWLLHP
MATVVLPKSTVFSLNHLVYSHV
(SEQ ID NO: 81)
MASPCLQKLPAVEHLFALTPEIPFQRHPEYMSITKEANEWAFKCMRRDFS
PEEKKCLVQWKVPMFTCLSTPHAPKANMVASAKFAWLTAFLNDPFDDNEV
AAGALATSYLDTVLSLCYGTASLAEVPDILAYRACHDLMEDLRSLLKPEL
FKRTVSTVEGWARSISSDDLTQDYELYRRKNVFILPLIYAMGASFDDEDV
ESLDYIRAQNAMLDHMWMVNDVFSFPKEFYKKKFNNLPAVLLLTDPSVQT
FQDAVNTTCRMIQDKEDEFIYYRDILATNASRNGKKDFLKFLDVLSCTIP
ANLVFHYASSCYHGMDNPLLGGGTFRGTWILDPKRTIIVSDPKSQAIPHA
VHIWKSAVFYAQSYFIQSLED
(SEQ ID NO: 82)
MASLCLQKLPAVEHLFALTRFELPEIPCSLSFQRHPEYTSITKEANEWAF
KCMRRDFSPEEKKCLVQWKVPMFTCLSTPHAPKANMVASAKFAWLTAFLD
DPFDDNEVAGGALATSYLNTVLSLCYGTASLAEVPDILAYRACHDLMKDL
RSLLKPELFKRTVSTVEGWARSILSDDLTQDYELYRRKNVFILPLIYAMG
ASFDDEDVESLDYIRAQNAMLDHMWMVNDVFSFPKEFYKKKFNNLPAVLL
LTDPSVQTFQDAVNTTCRMIQDKEDEFIYYRDILATNASWNGKKDFLKFL
DVLSCAIPANLVFHYASSRYHGMDNPLLGGGTFRGTWILDPKCTIIVSDP
KRCNVVASSNKLNQIQNLSNLI
(SEQ ID NO: 83)
MPGEYSFYNFLDMGFAPYGDYWKNMRKLCATGTIPSRREKIGPYLLDSAR
RERWGFLPKRCDLTTTGSNIFPTQSNLCYGTASLAEVPDILAYRACHDLM
KDLRSLLKTELFRRTVSTVEGWARSILSDDLTQDYELYRRKNVFILPLIY
AMGASFDDEDVESLDYIRAQNAMLDHMWMVNDVFSFPKEFYKKKFNNLPA
VLLLTDPSVQTFQDAVNTTCRMIQDKEDEFIYYCDILASVPEWEESFPEV
PGCSLLRNPANLVFHYASSRYHMDNPLGGGTFCGTWILDPKRTIIMSDPR
RCNVVASSNKLNQIQNLSNLI
(SEQ ID NO: 84)
MAFVVEKIPAMEHHLGLKRFYLPPIRCSIPSSAWDPDHKLVAKLANEWAF
PFINPSMSDAQKLSLERMRIPLYMSMLVPCGSTESAVLCGKFAWFGTMLD
DLLEDESPGGAPREEFLETFQGILHGTHPHRDPVHPSLEFCADLIPRLRS
SMAPRVYAHWVAQMEAYAASMDRSVLSLAQSASTVESYLARRRLDCFLLP
CFPFIEMSLEIALPDSDLESRDYLALQNAINDHVLLVNDVISFPAELRAK
KPLRSIASLQLLLDPSVNTFQDSVDRTCAMIQEKEREVTHYYDVVMRNAV
ASGNAELVSYLQILKMCVPNNLKFHFISSRYGVNDAESGHGIWIVL
(SEQ ID NO: 85)
MGYVGVNMEVLVDCRNTVFAKGLTSLEELWWWCFGRHGFLTQCTLKRRLI
LSKGTCRQLSITNRPFSLYISWRVLPRHYIAYTALEKHRRRSIMGASSIL
SIFEGAKSFYIPPHSSYHVDLNPAYDAKLDAEIDKWCMDFLNLHDLTDHK
TQFAIQSKLGKLAGFAYQAISSERLSPIAKFFCWLFLADDFMDDPSVPVS
DLKNATLAYKLIFKNDYDQAITLVESKGLLRQMGMLNDVYTDLKGFMNPG
HKTRFSKSMIDVLDMFEVESSWLHKTLVPNFEIYMWMREVTAGVIPCMVA
MDFLNNFGLEEEGVLDDPHIQTLEVIANRHSFLANDMVSFKKEWACEQYL
NSVALVGYSSNCGLNEAMEKVAQMVQDLEKEFADIKQKVLSNKDLNKGNV
MGYVQSLEYFMAANIEFSWISARYHGVGWVSPAEKYGTFEF
(SEQ ID NO: 86)
MGPSSILSIFEGAKSFYIPPHSSYHVDLNPAKLDAEIDKWCMDFLNLHDL
TDHKTQFAIQSKLGKLAGLAYQAISSERLRPMAKFLCWLFLADDFMDNPS
VPVSDLKNATLAYKLIFKNDYDQAITLVESKDLLRQMGMLNDVYTDLKGF
MNPGHRTRFSKSMIDVLDMFEVESSWLHKKLVPNFEIYNVTAGVIPCMVA
IDFLNNFGLEDDVLDHPNIQRLEVIANRHTYLANDMVSFKKEWACDMYLN
SVALVGYSSNCGLHEAMEKVAQMVQDLEKEFADIKQKVLSNKDLNKGNVM
GYVQGLEYFMAGNIGSLRDIMGWDGFHQLRNMVPWSSSLLLLALEAGA
(SEQ ID NO: 87)
MAAPSIYRPQILEQLLACKSIYLPQIRCSLPLQCHPDYASVSRQANDWAF
RFLKINATNAAAEKKCFTQWRTPLYGTFVVPWGDSRHALAAAKYTWLITI
LDDAVDEEPSQRNEILEAYMSLASGNLLAATRTKRSSRKSLNAVHRREDF
VVKPMLNFTQMCLGVKLRDKDLESEEYLRAIDAMFDHIWLVNDIFSFPKE
LRKKTFKNIIFLLLFTDHTVRSVQQAVDKANAMVQEKEQEFMYYHEILTR
KAMESGNHDFLAYLRAIPAFIPGNLRWHYLTARYHGVDNPFVTGEPFSGT
WLFHDTQTIILPEYKPTHPHLQV
(SEQ ID NO: 88)
MAAPSIYRPQILEQLLACKSIYLPQIRCSLPLQCHPDYASVSRQANDWAF
RFLKINATNAAADKKYFTQWRMPLYGTFVVPWGNSRHALAAAKYTWLITI
LDDAVDEEPSQRDEILEAYMSLASGQRSIAQVPNKPVLVAQAELVPDLRK
LMSPLLFQRLLVSYRKFVGCYSAKVDEEEFTKESYAVHRREDYVVKPMLN
FTQMCLGVELRDKDLESEEYLRAIDAMFDHMWLVNDIFSFPKELRKKTFK
NIIFLLLFTDHTVRSVQQAVDKANAMIQEKEQEFMYYHEILTRKAMESGN
HDFLAYLRAIPAFIPGNLRWHYLAARYHGVDNPFVTGEPFSGTWLFHDTQ
TIILPEYKPTHPHLQV
(SEQ ID NO: 89)
MGMLNDVYTDLKGFMNPGHKTQFSNSMIDVLDMFEVESSWLHKKLVPNFE
IYMWMREEWACEQYLNSVALVGYSSNCGLNKAMEKVAEMVQDLEKEFADI
KQKVLSNKDLNKGNVMGYVQSLEYFMAANIEFSWISARYHGVGWVSPAEK
YGTLEF

While this disclosure has been described with an emphasis upon particular embodiments, it will be obvious to those of ordinary skill in the art that variations of the particular embodiments may be used, and it is intended that the disclosure may be practiced otherwise than as specifically described herein. Features, characteristics, compounds, chemical moieties, or examples described in conjunction with a particular aspect, embodiment, or example of the invention are to be understood to be applicable to any other aspect, embodiment, or example of the invention. Accordingly, this disclosure includes all modifications encompassed within the spirit and scope of the disclosure as defined by the following claims.

We claim:

1. An isolated nucleic acid molecule, comprising a nucleic acid sequence that encodes a terpene synthase protein at least 80% identical to a protein encoded by the nucleic acid sequence according to any of SEQ ID NOs: 1-47 or a degenerate variant thereof or a functional fragment thereof.

2. The isolated nucleic acid molecule of claim 1, further comprising a promoter operably linked to the nucleic acid sequence that encodes the terpene synthase protein.

3. The isolated nucleic acid molecule of claim 1, wherein the isolated nucleic acid comprises the cDNA set forth as anyone of SEQ ID NOs: 1-47, or a degenerate variant thereof.

4. A construct comprising isolated nucleic acid molecule of any one of claim 1.

5. The construct of claim 4, wherein the construct confers an agronomic trait to a plant in which it is expressed.

6. The construct of claim 5, wherein the agronomic trait comprises terpenoid production.

7. An expression vector comprising the nucleic acid molecule of claim 1.

8. A host cell transformed with the vector of claim 7.

9. The host cell of claim 8, where the cell comprises a prokaryotic cell or a eukaryotic cell.

10. The host cell of claim 8, wherein the cell comprises a single cell organism.

11. The host cell of claim 9, wherein the prokaryotic cell comprises a bacterial cell.

12. The host cell of claim 10, the single cell organism is yeast.

13. The host cell of claim 9, wherein the eukaryotic cell comprises a plant cell.

14. A transgenic plant stably transformed with the isolated nucleic acid molecule of claim 1.

15. The transgenic plant of claim 14, wherein the plant is a dicotyledon or a monocotyledon.

16. A seed of the transgenic plant of claim 14.

17. A method of producing a transgenic plant comprising transforming a plant cell or tissue with the construct of claim 1.

18. A method for producing terpenes, comprising:
transforming a cell with the isolated nucleic acid molecule of claim 1; and isolating the terpenes produced from the cell.

19. The method of claim 18, where the cell comprises a prokaryotic cell or a eukaryotic cell.

20. The method of claim 18, wherein the cell comprises a single cell organism.

21. The method of claim 19, wherein the prokaryotic cell comprises a bacterial cell.

22. The method of claim 20, the single cell organism is yeast.

23. The method of claim 19, wherein the eukaryotic cell comprises a plant cell.

24. A plant cell, fruit, leaf, root, shoot, flower, seed, cutting and other reproductive material useful in sexual or asexual propagation, progeny plants inclusive of F1 hybrids, male-sterile plants and all other plants and plant products derivable from the transgenic plant of claim 14.


SEQUENCE LISTING (97 sequences)

SEQ ID NO: 1 - DNA, 1050 bp, Selaginella moellendorffii
atggctattctatccattgtgagcatttttgcagcggagaaaagctactccattccaccagcaagtaataaacttctggcctctccagcgctgaatccgctgtatgatgcaaaggccgacgctgagatcaatgtgtggtgtgacgagtttctgaagttgcaacctggaagcgagaaatctgtgtttattcgagagagcaggcttggattgctcgcagcttatgcatacccgagcatttcatacgagaagattgttcccgttgcaaagttcatcgcttggctctttcttgcagatgacattctggataaccctgagatctcttcgtcggacatgaggaacgtggcaaccgcatacaagatggttttcaagggaagatttgacgaggccgcacttctggtcaagaatcaggagctgctgaggcaagtgaagatgttatctgaggttttgaaagaactgtccctccatctagtggacaaatccggccgattcatgaattctatgaccaaggtgctcgacatgtttgagattgaatcgaactggcttcacaagcaaatcgttcccaacctggacacgtacatgtggctgagagagatcacatctggtgttgcgccttgctttgctatgcttgatggtttactgcaacttgggctggaagagcgtggcgtgctggatcatcctctcatacgcaaggttgaggagattgggacgcaccacattgcgctccacaatgacttgatctcgttcaggaaggagtgggcgaaagggaactacctcaacgccgtgcccattctcgccagcattcacaagtgtggtttgaacgaggcgattgccatgttggcgagcatggtggaggatttggagaaggagttcatcgggacaaagcaggagatcatttcaagtgggcttgccaggaagcaaggcgtcatggattatgtgaatggggtagaggtgtggatggccacaaacgcagaatggggatggttgagtgctagataccatggaattgggtggatccctcctccagaaaaatcagggaccttccaactctag

SEQ ID NO: 2 - DNA, 1077 bp, Selaginella moellendorffii
atggctttagctctagacaagatctatgctattgagaagttgctaggcctcaagaatttccacctcccaaagatcccttgctccattccttcagtcccttgccatccagatagcatctatgcatccaacaaggcccatgaatgggcatacaagttcatggatccaaaaatgacagccgctgatagaaaggctttggaagattggaaaatcccaatgtttgcaaccctcgtagtgccatttggatccaagagaaatgctgtcatttgctcaaagtatagcatgtttgccttattagtggacgactcggttgatgagggcttcgttgaaagtaccattcttcaagattactattccacaatccttaatcacctccataatcctaatttcaagatccaggcatcggacgatcaccttccacttcgagtttacagggccactgaagagcttgttactgagataagatcatccatgcttcctccagtgtatgctcattttgtagcacagtttgagaggtatgcactcagcagaatggcaagcaggcccaagtttcaatctgtcaagcagtatatcgaatggagaaggtttgatgtgttcttagagcctatcttcagcttcatagagatggcacttgaagtcgcagttccggacacggaactggaatcagaggactatctaattctgcgagatgctggaattgactatatatctatgtacaatgatgttctctcgtttgcaaaggagtttgcatgcaacaaactgctgaaccttccagtgttgctgcttctttcggatccggaggtggagtcattccagaatgcagtggacaagagttgcaagatgatcgtggacaaggagcaagaatttgtatactaccacaacattctgatcactcaggcaagaggtgaaggtaaacacgcgtttgtgaagtatcttgagtgtcttcctactgttctttccaacacgctttactatcactactcctctgcccgttaccatccagctttcataacgggtgagaagtttgatgcgaattggtgcttggacactgttataaaccatagaagaactggccggtga

SEQ ID NO: 3 - DNA, 1101 bp, Selaginella moellendorffii
atggccgcaccttctatctatcgtccccaaattctggagcagctcctcgcctgcaagagcatctacttgcctcaaattcgctgctcgttgccattgcagtgccacccagactacgcctccgtctccaaacaggcgaacgattgggccttccgcttcctcaagatcaatgccaccaatgccgctgccgataagaaatacttcacccagtggaggatgccactctacggcacctttgttgtgccttggggcgactccaggcacgctctagcggccgccaagtacacctggcttatcaccattctcgacgatgcggtcgacgaggagccttcgcagcgggacgagatcctggaagcttacatgagccttgcctccggtcaaagatccatcgcccaagttcccaacaagcccgtgctcgtcgcccaagccgagctcatcccggatctgcagaagctcatgtcgccgctcctcttccagcggctgctcgtctcgtacaggaaatttgttggctgctactcggccaaagtcgacgaggaggagttcacgaaagagtcttacgctgtgcatcgccgggaggactacgttgtcaagccgatgcttaacttcacgcagatgtgcctgggagtcgagctgagagacaaggatctggaaagcgaggagtacctgcgggcgatagatgccatgtttgatcatatgtggctggtgaacgacatcttttcattcccaaaggagctgaggaagaaaactttcaagaacataatttttctcttgctcttcacggaccacaccgttcgctctgttcaacaggcagtcgataaggcgaacgccatgattcaggaaaaagaacaagaattcatgtattaccacgagatcctgacgaggaaagcaatggaatctggcaaccacgactttctggcgtaccttagagcgattccggcattcatccctggaaatctacgttggcactacctcacagctcggtaccacggtgttgataatccatttgtaacaggagagccattcagtgggacttggttgtttcatgatacgcagactatcatactccccgagtacaaaccaactcatccccatctgcaagtctga

SEQ ID NO: 4 - DNA, 1101 bp, Selaginella moellendorffii
atggccgcgccttctatctatcgtccccaaattctggagcagctcctcgcctgcaagagcatctacttgcctcaaattcgctgctcgttgccattgcagtgccacccagactacgcctccgtctccaaacaggcgaacgattgggccttccgcttcctcaagatcaatgccaccaatgccgctgccgataagaaatacttcacccagtggaggatgccactctacggcacctttgttgtgccttggggcgactccaggcacgctctagcggccgccaagtacacctggcttatcaccattctcgacgatgcggtcgacgaggagccttcgcagcgggacgagatcctggaagcttacatgagccttgcctccggtcaaagatccatcgcccaagttcccaacaagcccgtgctcgtcgcccaagccgagctcatcccggatctgcagaagctcatgtcgccgctcctcttccagcggctgctcgtctcgtacaggaaatttgttggctgctactcggccaaagtcgacgaggaggagttcacgaaagagtcttacgctgtgcatcgccgggaggactacgttgtcaagccgatgcttaacttcacgcagatgtgcctgggagtcgagctgagagacaaggatctggaaagcgaggagtacctgcgggcgatagatgccatgtttgatcatatgtggctggtgaacgacatcttttcattcccaaaggagctgaggaagaaaactttcaagaacataatttttctcttgctcttcacggaccacaccgttcgctctgttcaacaggcagtcgataaggcgaacgccatgattcaggaaaaagaacaagaattcatgtattaccatgagatcctgacgaggaaagcgatggaatctggcaaccacgactttctggcgtaccttagagcgattccggcattcatccctggaaatctacgttggcactacctcgcagctcggtaccacggtgttgataatccatttgtaacaggagagccatccagtgggacttggttgtttcatgatacgcagactatcatactccccgagtacaaaccaactcatccccatctgcaagtctga

SEQ ID NO: 5 - DNA, 777 bp, Selaginella moellendorffii
atggctccctacgatttcgttccaaatgtgcagtgttcgttccctgtgaagtgccaccctctgtattctttcattcgtccaggcttggaagattgggctgcaactttggagcctgggcatggtgaagggaacccgaaaggcctgggagctgacttgggaggtgccaagaggcttgttgatagctaccttggcataatccatgccccggaacccgtggcagatatggaatttccacggttctgtgatatgtggaatgatctacgtgcagatatgccactcaagcagtaccagcgatttgccaacagagtgtccgagctgttgaaggcaagtgtgaatcaggtgaggctaaggaatctgaaaacggtgatgggcttggaggagctgctggctcaccgtcgcatgttagttggtgtatttgttatggaaactctaatggagtatggcatgggattcgaactccaggacgacgccatttcaaatcaggacctccaagaggctgaaagtctggttgcagaccactgcaactggcgatactcagttcaatatcgtccatgcggcaattcggattcaaagggcttttctttcgagtatgcagccgacaaagttcaaaaactggtgcagagtatcgagcatcgattcaagaagctgtgcgagaatatcagaagatcaagctgctacaatggtgcaatggaggcttacctggaaggcttgtctcatattatatccggaaaccttgagtggcaccggcagacaggacgatacaaactggtatcttga

SEQ ID NO: 6 - DNA, 1272 bp, Selaginella moellendorffii
atggctgccagcgtcaatggcgtgctcccggagctctccactctctcaaaatttgaactccgtccattgccctgcgcgtttcctttcgagtgccacccaaatcacgcgtcgctcacccgagaggttgacgagtgggcgatccgatcgctgcaagcccggggctccatgcccaagcgccagatgatcatcgagtccaagatctcggcggcggcatgcatgactatcccgcgtggccgggacgatcgtaagatggtgctggcgggcaagcatttgtgggcgctcttcttgctggacgacgcgctggaatcgtgccggagccaggaggccgcgagagtcctcgcccggcgagcgatggaagtcgcgagaggggaccaattggaagggatgatccaggaggaaagagaactagaagaagccaaaggggtcgcgaggaaattcgcgatccaggaagaagaaggagatcgatataatgatcagtcgagaggaatccttgcaaacatagcgatccaagaggaccctggtctcatcgatctggctaccagaggaatggcgacgaaaatcgcaatcacggaagaagatcaaggtcgcgattctcgatgggcgctgggattgttccgggaagtagtggcggagctccggcgatcaatgccgctcccgatgttcgatcgctacctgcggtacctggatcgctacctggaggccgtgatccaggaggtgggataccagatcgcgggccacatcccgcgggaggacgagtatcgcgagctccggcggggaacgtccttcacagagggcaccagcgcgatctttggcgagctgtgcatggggctggagctccacgaatctgtgacatcgtctcgcgatttcatcgaattcgtggcgctcgtcgcggaccacatcgcgctcaccaacgatgtcctctccttccgcaaggatttctacgccggggtcgcccacaactggctcgtcgtgctcctccgccacagccaccgcgggaccggcttccaatccgcgctggacagcgtctatggcatgatccgcgacagcgagtgccggatcctggggctccagtcgcgaatcgaggcgcaagcactgaagagtggcgatggtcacctcctcagcttcgcgcaggcgtttcccctgtgcctggccgggaatcggaggtggtcatcgatcaccgcgcgataccatggcattgggaatcctctcatcactggcgtggagttccacgggacatggctcttacatccggatgtcaccatagttatttga

SEQ ID NO: 7 - DNA, 1007 bp, Selaginella moellendorffii
atggcacttgccgtggagaagattcccgccatggaacacctcctggggctaaagaggttctatttacggcccattcgctgctccatcccctccagcgcctggcatcccgaccacaagctcgttgccaagctcgcgaacgagtgggcattcccattcatcaatcccagcatgagcgatgcccaaaagctctccctggagcgcatgcgaatcccgctctacatgagcatgctcgtgccgtgcggatccaccgagttcgcgtggtttggaacgcgatcatgctggatgatctcctcgaggacgagtcccccagcggcgccccccgggaggagttcctggagactttccagggcatcctccacggggcgcacccacatcgcgatccagtccatccatcgctcgagttctgcgcggacctcattccgcgcctgcgatcatccatggctccccgggtgtggtcgcgcagatggaggccctacgctgcctccatggaccggagcgtcctttctctagcacaatcggcgttgacggtcgagcccgcaggaggctcgattgcttcctcctcccctgcttcccattcatcgagatgtcgctggagattgcgctcccagacagcgatttggagtcgcgggactacctggcgctccagaatgccatcaacgaccacgtcctccttgtcaacgacgttatctcctttcccgcggagctgcgcgccaaaaagccactgagaagcatcgcgtccttgcagttgctcttggattcccagcatcaacacgctccaggaatcggtggacagaacctgtgcgatgatccaggagaaggaacgcgaggtgacgcattactacgacgttgtgatgagaaacgctgtggcttctggcaatgccgagcttgtgagctaccttgagatcctcaagctgtgcgttccaaacaacctcaaggtccacttcattagttctcgttatggagtgaatgatggcgagtctggtcatggaatttggattgttctgtag

SEQ ID NO: 8 - DNA, 1041 bp, Selaginella moellendorffii
atggcacttgctgtggagaagattcccgccatggaacacctcctggggctaaagaggttctatttacggcccattcgctgctccatcccctccagcgcctggcatcccgaccacaagctcgttgccaagctcgcgaacgagtgggcattcccattcatcaatcccagcatgagcgatgcccaaaagctctccctggagcgcatgcgaatcccgctctacatgagcatgctcgtgccgtgcggatccaccgagagcgccgtcctctgcggcaagttcgcgtggtttggaaccatgctggatgatctcctcgaggacgagtcccccggcggcgccccccgggaggagttcctggagactttccagggcatcctccatgggacacacccacatcgcgatccagtccatccatcgctcgagttctgcgcggacctcattccgcgcctgcgatcatccatggctccccgggtgtacgcgcactgggtcgcgcagatggaggcctacgctgcctccatggaccggagcgtcctttctctagcacaatcggcgtcgacggtcgagagctacttggcgcgcaggaggctcgattgcttcctcctcccctgcttcccattcatcgagatgtcgctggagattgcgctcccagacagcgatttggagtcgcgggactacctggcgctccagaacgccatcaacgatcacgtcctccttgtcaacgacgttatctccttccccgcggagctgcgcgccaaaaagccactgagaagcatcgcgtccttgcagttgctcttggatcccagcgtcaacacgttccaggactcggtggacaggacctgtgcaatgatccaggagaaggaacgcgaggtgacgcattactacgacgttgtgatgaggaacgctgtggcttctggcaatgccgagcttgtgagctaccttcagatcctcaagatgtgcgttccaaacaacctcaaggtccacttcattagttctcgttatggagtgaatgatggcgagtctggtcatggaatttggattgttctgtag

SEQ ID NO: 9 - DNA, 480 bp, Selaginella moellendorffii
atgtcgagctttcggagtctcatggatcccccactgtttgctcgctatatgatttgtttgaaaacatttctggattctttggtggaggaggcctctttgcgatctgccaaatccatcccaagtctcgagaaatatcagttgctccggagagggacagttttcgttgaaggagccggagattttgtagcatttgtcaacgcagtggctgatcatgttctcttctccttccgacacgagatgaaaatcaagtgcttccacaactatctctgtgtcatcttttgccacagcccgaataatgcaagctttcaagaggctgtcgacaaagtatgcaaaatgatccaggagaccgaagccaagatccttcaactccaaaagaagctgatgaagatgggcgaggaaactgggaacaaagacctggtggactatgcaacatggtatccttgttttacatctggacatctccgctggccatgggcttga

SEQ ID NO: 10 - DNA, 627 bp, Selaginella moellendorffii
atgtcgagctttcggagtctcatggatcccccactgtttgctcgctatatgatttgtttgaaaacatttctggattctttggtggaggaggcctctttgcgatctgccaaatccatcccaagtctcgagaaatatcagttgctccggagagggacagttttcgttgaaggagccggaggcattatgtgtgagttttgcatggatctcaagctggataaggtggctgatcatgttctcttctccttccgacacgagatgaaaatcaagtgcttccacaactatctctgtgtcatcttttgccacagcccgaataatgcaagctttcaagaggctgtcgacaaagtatgcaaaatgatccaggagaccgaagccaagatccttcaactccaaaagaagctgatgaagatgggcgaggaaactgggaacaaagacctggtggactatgcaacatggtatccttgttttacatctggacatctccgctgggtgtatgtcacaggacgctaccatgggcttgacaatccgctgctgaacggtgaaccattccatgggacttggtttctacatccagaagccaccttcattctaccattcggatccaaatgtggatttattaacaccatgtga

SEQ ID NO: 11 - DNA, 1155 bp, Selaginella moellendorffii
atggcattgcccagcctgctctcaacaaagctcaagccgcttgagctcctgtccggtgtcactcattatgatcttccgccaattccctgttctcttcctgtcaagtgtcatcctcaatttgctaagttttctcgcattgccgatacatgggccatcgacgcaatgcagctgcaaaatgatccatgtggaaagctcaaggctgtgcagagccgagccccgctgctttactgcttcctcgtccctttcggcatcggagaggaagaaatgattgcaggctgcaagtacagctggtcgacttccttcgtggatgatccatttgacgaagaaacggatttgaagcgggccaaggaatggaagaaggtcgtgctgcgagctgcgaacggtactcccagtgctgaagatttgatgataaggacgataaaagcttattcggagattatgatgcacctgcaacagatgatggcggccccagtgttttcgaggttcatgagggctcactacgcttgggcagatcactgcgtggagcttgttcgtagaaggcagcataaagaccctccaactgtagccacataccttgcagacaggtgcgaaaatctgctcgtagaaccgatcttcattctggcggaggtgtgcatgaagcttcagattgacccggagttcctgtcgctgccagagttcaagaaaatctggaccacaatgctggaacatgcggctatcgtgaacgacgtcttgtcaatccgtgtagacatcctcaacggacactactacacctatcctggcctcgtcttccagcagcatcctgagatccaaactttccaggaggctgtggactattccgtggggatgatccagaccaaggagagaaagttcatcaaactgcacgagatgctgaccgacaaagccaggcaatgcggcttcaagaacaagtccgacttgctcaagtatgttgaagctttgccaaacttcatcgctggaaatctttactggcactaccttagcgccagatacttcggtgtcaacaaccccttcctcaccggagagcctgtccaaggcaccatcctcatccatccccgcaacacagtcgtgctcccaccttaccagcgaaacaagcacccctttctcatcgatgtcgacaatctggagctcggtgcgtga

SEQ ID NO: 12 - DNA, 1077 bp, Selaginella moellendorffii
atgaggagcttcagcagcttccacatctccccaatgaaatgcaagcctgcattgcgagtccatccattgtgtgacaagctccagatggaaatggaccgctggtgtgtagacttcgcttcgccagagtcctcggacgaggagatgaggtcctttatagctcagaagctgccctttctctcgtgcatgctcttccccacagcgctcaactcaaggatcccatggctgatcaagttcgtatgctggttcacactgttcgattcgctcgtcgacgacgtcaagtccctgggcgcgaatgcccgagacgcgtcggcgttcgtgggtaagtaccttgaaaccatccatggagctaaaggggcgatggcgccggtgggaggctcgctcctctcgtgcttcgcctcgctgtggcaacacttccgcgaggacatgccgccgcggcagtactcgcgcctggtgcgccacgtgttgggcctgttccagcagtcggcttcgcagtcccggctccgccaagagggcgccgtcctcacggccagcgagtttgtggccgggaagcgcatgtttagctcgggggcgacgctggttttgctcatggagtatggactgggggtggagctcgacgaggaagtgctcgagcagccggccatccgggacattgcgacgaccgccatcgaccacctcatctgcgtcaacgacatcctccccttccgtgtggagtatctctccggggacttctccaacctcctctcctcgatttgcatgtcccagggcgtcggattgcaagaggcggcggaccaaactctcgagctgatggaggactgcaaccggaggttcgtggagctgcacgacttgataaccaggtcgagctacttctccaccgctgtggagggctacattgacggccttggctacatgatgtccgggaaccttgagtggagctggttgactgcccgctaccatggtgtggactgggtagcgccaaacttgaaaatgcggcagggggtgatgtaccttgaagaaccaccacgttttgagccaactatgccactagaagcttacatttcgtctagtgattcttgctag

SEQ ID NO: 13 - DNA, 1101 bp, Selaginella moellendorffii
atggaggctattgtttcatccagcaagatccatgcagtagaacatttgctgagcctcaagagctactctctccctcaaatcctccttgcccatcccgtcaagtgtcaccccgactacacctcgatctgcaaggaatcggacgaatggatcttcagctacctcggcgtcacaagcccggaacacaagaagcgcttagcgcaatggagggtcccaatcttcgccgccttcctgacgccccccagcagccccaagaggcgcacgcttttgggcggcaaatttacgtggctgatcactgcgctggatgatcagctggacgagagcaagatctcccaagctgggcggagctgccagtacagggacgccatcttgagcattttctccggcagaagcgattacccggccatactcccggcggaggttcctcttctcagagcctgcgaggagctcatgccggaaatccgctccttcatgcttccgcccactctcaatcgcttccttgcttacaccaagcagtggtcgcagactttcgatgttgcctatgagagcacacaagtgttcaaggagctaaggagggacaacgtttggatcaccgcatacttcccgatgatcgagatgttcctgggattgggtcttggggacgatgtggccgggtccaaggatttcctcgccgctcaggacgcaatatcggaccatgcctggatggtgaacgacctcttctctttcgccaaagagttccgggacgagaaaaagctcagtaacattctgtccgtgagcttgctcatggattcgtgcgtgcacaccatccaggacgccattgatcttctgtgtaccgagttgcaagcaaaggaggaggagtttctctactaccacgggatccttgtcaagcgagcccaagcagggaacaaccaagatctcttaaggtacctagaggcaatccttgccgtgatcccaggtaatttacactttcactacataacggcgcgttaccacggatacaataatccatgtgtaaatggagaagcatggcatggtaaagttatattgcaaccaaatactctcgggccaccaccaaagccacatccatacctctatgacatataa

SEQ ID NO: 14 - DNA, 1047 bp, Selaginella moellendorffii
atggctgtttcatccattgtgagcattttcgcagcagagaaaagatattccattccaccagtgtgtaaactccttgcctctccagtgctgaatcctctgtacgatgcaaaggccgagtctcagatcaatgcgtggtgcgccgagtttctgaagttgcaacctggaagcgagaaagctgtgtttgttcaagaaagcaggcttggattgctcgcggcttatgtttacccgaccattccatacgagaagattgttccggttggaaagttcttcgcttggtactttcttgcagatgacattctggatagcccggagatctcctcgtcggacatgaggaatgtgacaactgcatacaagatggttttaaagggaaaattcgacgaggccacgcttccagtgaaaaatccggagctgctgaggcaaatgaagatgttatctgaggtcttggaagaattgttcctccatatagtggatgaatcaggccgattcgtggatgctctgaccaaggtgctcgacatgtttgagattgaatcgagctggcttcgcaagcaaatcattcccaacttggatacgtacctctggctgagagagatcacatctggtgttgctccttgctttgctctgattgatggtttactgcaacttaggctggaggagcgtggcgtgctggatcatcctctcatacgcaaggttgaggagattgggacgcaccacattgcgctccacaatgacatgatctcgttcaggaaggagtgggcgaaaggatactacctcaatgccgtgcccattctcgccagcaattgtaagtgtggcttgaacgaggcaattggcaaggttgcgagcatggtggaggatgtggagaaggatttcgcccagacaaagcatgagatcgtttcaagtgggcttgccatgaagcaaggagtcatggactatgtgaacgggatagaggtgtggatggccggaaacgtagaatgggcatggacgagcgctagataccatggaattgggtggatccctcctccagaaaaatcagcgaccttccaactctag

SEQ ID NO: 15 - DNA, 1590 bp, Selaginella moellendorffii
atggctcgcacgttattcaacgacatgctcaagcaggcagccctccctgacatcgtcactttctcgacactagtggaaggatactgtaatgctggactggtggatgacgccgagaggcttctggaagagataattgccagtgactgctctccggacgtgtatacgtacacaagcctggtcgacagcttctgcaaagtcaaaagaatggtggaggcgcacagagttctcaagcgaatggccaagcggggatgccaacccaacgtggtgacttacactgctctcattgacgcgttctgcagagctgggaagccgacggtggcttacaagctgctggaggagatggttggcattaacaacgacgtccagccgaacgttcaggagctggcttctgtgggactggggacctggaagaggctcgcaagatgctcgagagactggagcgcgacgagaactgcaaggcggatatgttcgcatacaggggggctgtgccaggggaaagagctcagcaaagccatggaagttttggaagagatgacgctctcaaggaaaggcaggccaaatgccgaggcttacgaggcggtgatccaggagctagcgagagaagggaggcatgaagaggcaaacgcgctcgcagacgaattactgggtaacaaaggccaccttctttcagtgtttaaaattcacttaggaagcatccattgcgagcattttcgcagtggagaaaagctattccattccaccaaaagcaggcttggattgctcgcggcttatgtttacccgaccattccatacgagaagattgttccggttgcaaagttcatcgcttggttctttcttgcagatgacattctggatagcccggagatctcctcgtcagacataagatatgtggcaaccgcatacaagatggttttcaagggaagatttgacgaggccacacttccagtgaaaaatccggagttgctgaggcaaatgaagatgttagctgaggttttggaagaactgtccctccatatagtggatgaatcaggccgattcgtggatgctatgaccaaggtgctcgacatgtttgagattgaatcgagctggcttcgcaagcaaatcattcccaacctggatacgtacctctggctgagagagatcacatctggtgtggctccttgctttgctctgattgatggtttactgcaacttaggctggaggagcgtggcgtgctggatcatcctctcatacgcaaggttgaggagattgggacgcaccacattgcgctccacaatgacttgatcttgctcaggaaggagtacttcctggcaagtgactatgatgttgatttgccttcatcggaggcaagcagcactctgttttttcttttgcaaatggctactttcatgaaatactttttagaggatctatgcagccattttgccgctcgctgccggataatcccatacaagaatgtctcgagcctgtggatggatcaatctggggcggtgctccagaagaagctcttgaagctcgagttcactacgctctttgagtacctccaacggctgtctccgacttctacatcccctggaactccatggtaa

SEQ ID NO: 16 - DNA, 1047 bp, Selaginella moellendorffii
atggctgtttcatccattgcgagcattttcgcagcagagaaaagctattccattccaccagtgtgtcaactccttgtctctccagtgctgaatcctctgtacgatgcaaaggccgagtctcagatcgatgcgtggtgcgcggagtttctgaagttgcaacctggaagcgagaaagctgtgtttgttcaagaaagcaggcttggattgctcgcggcttatgtttacccgaccattccatacgagaagattgttccggttggaaagttcttcgcttcgttctttcttgcagatgacattctggatagcccggagatctcctcgtcggacatgaggaatgtggcaactgcatacaagatggttttaaagggaagatttgacgaggccacgcttccagtgaaaaatccggagctgctgaggcaaatgaagatgttatctgaggtcttggaagaattgtccctccatgtagtggatgaatcaggccgattcgtggatgctatgaccagggtgctcgacatgttcgagattgaatcgagctggcttcgcaagcaaatcattcccaacctggatacgtacctctggctgagagagatcacatctggtgtggctccttgctttgctctgattgatggtttactgcaacttaggctggaggagcgtggcgtgctggatcatcctctcatacgcaaggttgaggagattgggacgcaccacattgcgctccacaatgacttgatgtcgctcaggaaggagtgggcgacaggaaactacctcaacgccgtgcccattctcgccagcaatcgtaagtgtggcttgaacgaggcaatcggcaaggttgcgagcatgctgaaggatttggagaaggatttcgctcggacaaagcatgagatcatttcaagtgggcttgccatgaagcaaggagtcatggactatgtgaacgggatagaggtgtggatggccggaaacgtagaatggggatggacgagcgctagataccatggaattgggtggatccctcctccagaaaaatcagggaccttccaactctag

SEQ ID NO: 17 - DNA, 1191 bp, Selaginella moellendorffii
atggctgtttcatccattgcgagcattttcgcagcggagaaaagctattccattccaccagtgtgtcaactccttgtctctccagtgctgaatcctctgtacgatgcaaaggccgagtctcagatcgatgcgtggtgcgcggagtttctgaagttgcaacctggaagcgagaaacccgtgtttgttcaagaaagcaggcttggattgctcgcggcttatgtttacccgaccatagattgttccgatgacattctggatagcccggagatctcctcgtcggacatgacgaatgtggcaactgcatacaagatggttttaaagggaagatttgacgaggccatgcttccagtgaaaaatccggacctgctgaggcaaatgaagatgttatctgaggtcttggaagaattgtccctccatgtagtggatgaatcaggccgattcgtggatgctatgaccagggtgctcgacatgtttgagattgaatcgagctggcttcgcaagcaaatcattcccaacctggatacgtacctctggctgagagagatcacatctggcgtggctccttgctttgctctgattgatggtttactgcaacttaggctggaggagcgtggcgtgctggatcatcctctcatacgcaaggttgaggagattgggacgcaccacattgcgctccacaatgacttgatgtcgctcaggaaggagcgggcgacaggaaactacctcaacgccgtgcccattctcgccagcaatcgtaagtgtggcttgaacgaggcaatcggcaaggttgcgagcatgctggaggatttggagaaggatttcgctcggacaaagcatgagatcatttcaagtgggcttgccatgaagcaaggagtcatggactatgtgaacgggatagagtgttttagaaattcatatctaagcagtgttttcgacctgaacaagcaaattgaaatgcacggtagatgtggtaacatcaaacacgcagctcaaatcttccatgcaagttgctgtgattttccttcatgggaggcaagcagcactctgttttttcttttgcaaatgccattttgccgctcgctgccggataatccctgggcggtgctcctgaagaagctcttgaagctcgagttcactacactctttgagtacctccaactgacttctacgtcccctggaactccatggtaa

SEQ ID NO: 18 - DNA, 1107 bp, Selaginella moellendorffii
atggaggccactttgatctccaaattctccactgtcacgcacttcgagcttccgcagcttcccaacaacatcccattcgcctaccacccgcaatccgcgacgatcagtgcccagatcgacgagtggatgcttcgcaagatgaagatcactgaccagagtgcgaggaagaagatgatccactccaagatgggactgtacgcctgtatgatgcatcccaatgccgagagggagaagctcgtcctggccggtaagaatctctgggccctcctcctcatggacgatttgctcgaatccagcagcaaggaagagatgcctcggctcaacaccaccatctccagccttggcagtggaaattccggggatggagctatccggaatcctgtgctgcttctgtataaagaagttctgggagagcttcgagctgccatggagccacctttgctggaccgctacttgcactgcctggcagcttcactcgaaggcgtccggaagcaagtccaccaccgaaccaagaagagcgtccctggaccagaagaatataagttcacccgtcgtgccaatggattcatggacatcctcgggggcatcatgaccgagttctgtatgggaatccgcctcaaccaagctcaaatccagtctccaaccttccgggagctcctcaactctgtgtctgattacgtcattctcgtcaatgacctgctgtccttccggaaggagttttacggtggcgattatcaccacaactggatctcggtactctcgtaccatggcccctccggaatcagctttcaggatgtgattgaccagctgtgtgagatgatccaagcagaagagcactcaatcctggccttgcagaagaagattgccgacgaagaaggttgcgactcggagctgacgaagttcgcaagtgagctagcaatggttgcttccgggagcctcgtgtggtcgtatctctctggccgctaccatggctatgataatccactgatcactggggagattttcagtggaacatggctgctgcatcccgtggccaccgtcgtcttaccatccatcaaggctcgagatacattgctggggctcaaagttccggttccactgccttga

SEQ ID NO: 19 - DNA, 1101 bp, Selaginella moellendorffii
atggaagatgttctagtttccagaattttgggtgtcacccatttcgagctcccattgcttcccaacaacattgcattttattgccacccggaattccaatcaatcagcctccaaatcgacgagtggttccttgacaagatgagaatcgccgacgagacttccaagaagaaggtgctggagtccaggatcggtttgtacgcctgtatgatgcatccccatgctgagagagagaagattgtgctggccgggaaacatctctgggccgtcttcctccttgacgatttgctggaatccagcggcacacaagagatgccgaagctcaacgccaccatttccgaccttgccagtggaaattccaacgaggatgttacaaatcctgtgttggttctctaccgagaagttatggaagagatccgggctggtatggagccaccattgctggatcgctacgtggagtgcctgggagcttcactggaagccgtgaaggatcaagttcaccaccgagccgagaaaagtatccctggagtggaagcttacaagcttgcccgccgtgccactggattcatggaagctgtcggcggtatcatgaccgagttctgtatgggaatccgcctcaacgaaagtcaaatccagtctccagtctttcgagagctcctcaattctgtgtctgatcacgttgttcttgtcaatgatctcttgtccttccggaaagagttctatgaaggtgcttgtcaccacaactggatctcagttctcctgcagcacagccccagcgggacgaggttccaggatgtcattgatcagctctgcgagatgatccaagaagaagagctctcaatcctggcattgcagaggaagatttccagtaaagaaaatagcgactcggagctgatgaagttcgcaagggagttcccaatggttgcttccgggagcctagtgtggtcgtatgtcactggccgctaccatggctatggtaatccgctgctgactggggagattttcagcggaacttggctgctccatcccatggccaccgtcgtcttgccaaagtctacagtcttttcattaaaccatttggtatattctcatgttattctttga

SEQ ID NO: 20 - DNA, 1143 bp, Selaginella moellendorffii
atggaagatattctagtttccagaatttcgggtgtcacccatttcgagcttccattgcttcccaacaacattgcattttattgccacccggaattccaatcaatcagcctccaaatcgacgagtggttccttgccaagatgagaatcaccgacgagacttccaagaagaaggtgttggagtccaggatcggtttgtacgcctgtatgatgcatccccatgctgagagagagaagattgtgctggccgggaaacatctctgggccgtcttcctccttgacgatttgctggaatccagcggcacccaagagatgccaaagctcaacgccaccatcttcaaccttgccagtggaaattccaacgaggatgtcacaaatcctgtgctggttctctaccgagaagttatggaagagatccgggctggtatggagccaccattgctggatcgctatgtggagtgcctgggagcttcactggaagccgtgaaggatcaagttcaccaccgagtcgagaagagtatccctggagtggaagaatacaagcttgcccgccgtgccactggattcatggaagctgtcgggggtatcatgaccgagttctgtatgggaatccgcctcaacgaaagtcaaatccagtctccagtctttcgagagctcctcaattctgtgtctgatcacgttgttcttgtcaatgatctcttgtccttcaggaaagagttctatgaaggtgcttgtcaccacaactggatctcagttctcctgcagcacagccccagagggacgaggttccaggatgcaattgatcagctctgcgagatgatacaagaaaaagagctctcaatcctggccttgcagaggaagatttccagcaaagaacatagtgactcggagctgatgaagttcgcaagggagttcccaatggttgcttccgggagcctcgtgtggtcgtacgtaactggccgttaccatggctatggtaatccgctgctgactggggagattttcagtggaacttggctgctccatcccatggccaccgtaaatggatatcaaaccattctagtatattctcttattaataatacggaaataaaatctataatctccacaatatatacagtttcccaaatcgcgagttctggttga

SEQ ID NO: 21 - DNA, 1107 bp, Selaginella moellendorffii
atgaaagatcttttcagaatttcaggtgtcactcatttcgagcttccgcttcttcccaacaacattccatttgcttgccacccggaattccaatcaatcagcctcaaaatcgacaagtggttccttggcaagatgagaatcgccgacgagacttccaagaagaaggtgctggagtccaggattggtttgtacgcctgtatgatgcatccccatgctaagagagagaagcttgttctcgccgggaaacatctctgggccgtcttcctccttgacgatttgctggaatccagcagcaaacacgagatgcctcagctcaacctcaccatctccaaccttgccaatggaaattccgacgaggattacacaaatcctctgctggctctctatcgagaagttatggaagagatccgagctgccatggagccaccattgctggatcgatacgtgcagtgcgtgggcgcttcactggaagccgtgaaggatcaagttcaccgccgagccgagaagagtatccctggagtggaagaatacaagctcgcccgccgtgccactggatttatggaagctgtcggcggaatcatgaccgagttttgcatcggaatccgcctcagccaagctcagatccagtctccaatcttccgggagctcctcaactctgtgtctgatcacgtcattctcgtcaatgacctgctgtccttccggaaggagttttatggtggcgactatcaccacaactggatctcggttctcttgcaccacagtccccgcgggactagtttccaggacgtagttgaccgcctgtgcgagatgatccaagcagaagagctctcaattttggccttgcggaagaagattgctgacgaagaaggaagcgactcagagctgactaagtttgcaagagagttcccaatggttgcttctgggagcctagtgtggtcgtatgtcactggccgctaccatggttatggtaatccgctgctgactggggaaattttcagtggaacttggctgcttcatcccatggccaccgtcgtcttgccatcgaagttcagaatggataccatgagattctctttagctccaaaaaaacgcgactcgtttccctga

SEQ ID NO: 22 - DNA, 1071 bp, Selaginella moellendorffii
atggaggccactttgatctccaaattctccactgtcacgcacttcgagcttccgcagcttcccaacaacatcccgttcgcctaccacccgcaatccgcgacgatcagtccccagatcgacgagtggatgcttcgcaagatgaagatcactgaccagagtgtgaggaagaagatgatccactccaagatcggactgtacgcctgtatgatgtatcccaatgccgagagggagaagctcgtcctggccggtaagaatctctgggccctcctcctcatcgacgatttgctcgaatccagcagcaaggaagagatgcctcggctcaacaccaccatcaccaaccttggcagtggaaattccagggatggagctatccggaatcctgtgctgcttctgtataaagaagttctgggagagcttcgagctgccatggagccacctttgctggaccgctacttgcactgcctggcagcttcactcgaaggtgtccggaagcaagtccaccaccgaaccagaaagagcgtccctggaccggaagaatataagctcacccgtcgtgccaatggattcatggacatcctcgggggcatcatgaccgagttctgtatgggaatccgcctcaaccaagctcaaatccagtctccaaccttccgggagctcctcaactctgtgtctgattacgtcattctcgtcaatgacctgctgtccttccggaaggagttttacggaggcgattatcacgacaactggatctcggttctctcgtaccatggccccagggggatcagctttcaggatgtgattgaccagctgtgtgagatgatccaagcagaagagcactcaatcctggccttgcagaagaagattgccgacgaagaaggttgcgactcggagctgacgaaattcgcaagtgagcttgcaatggttgcttccgggagcctcgtgtggtcgtatctctctggccgttaccatggctatgataatccactgatcactggggagattttcagtggaacatggctgctgcatcccgtggccaccgtcgtcttcccatccatcaaggctcgcccctga

SEQ ID NO: 23 - DNA, 537 bp, Selaginella moellendorffii
atgtttgaggacgtgatgttgagcattcaaagtctcatggatcccccactgtttgctcgctacatgatttgcttgagaaactatctggatgctttggtggaggactcctctttgcgatttgccaaatccattccaagcctcactaaacaccagctgctccggaagcagttggaggcattatatagagacaagcactacagctatctctgtgtcatcttttgtcacgataatgcaagctttcaggggaccgtggacaaagcatgtgaaatgatccaggagaccgaaggtgagatcttgcaactccaaaagaagctgatgaagctgggagaggaaactgggaacaaagatctggtggagtacgcaaggtacccttgtgttgcatctagaaaccttcgctggtcgtatgtcacacgaaccagcagtcgtgaaccattccatgcgacctggtttctacttccagaagtcaccctcatcgtgccattcggatccaaatgcggtgatcatccatttgccatcacagaaaatcatttggtttga

SEQ ID NO: 24 - DNA, 1110 bp, Selaginella moellendorffii
atggaagatgtcttggctgaaagattgtctagagttagcaagtttgatttgccttccattccttgcagcattcccttggaatctcatcctgaattctctcggatatctgaagttactgatgcatgggctattagaatgttgggtatcactgatccatatgagagacagaaagctattcaggcaagattcggtttgctcaccgcactagctacacctaggggagagagcagtaaactcgaggttgcatcaaagcatttctggacttttttcgttctagatgacattgccgagacagacttcggtgaggaagaggggcaaaaagctgctgatattttgcttgaagttgctgagggaagctatgtttttagtgaaaaagaaaagcagaagaatccgagctatgccatgtttgaggaggtgatgtcgagcttccgcagtctcatggatcccccactgtttgctcgctacatgacttgtctgaaaaactttctggattctgtggtggaggaggcctctttgcgatttgccaaatccattccaagccttgagaaataccagctgctccggagagagacagtctttgttgaagcgtccggaggtattatgtgtgagttttgcatggatctcaagctggataagggtgtggttgaatccccagaattcgtagcctttgtcaaggcagtggttgatcacgccgcccttgtcaatgatctcctttccttccgacacgagatgaaaatcaagtgcttccacaactatctctgtgtcatctttttccacagcccggataatgcaagttttcaagagactgtcgacaaggtatgcaaaatgatccaggagaccgaagctgagattttgcaactccaaaagaaggtgatgaagatgggcgtggaaactgggaacaaagatctagtggagtacgcaacatggtatccttgttttgcatctggacaccttcgctggtcgtatgtcacaggacgctaccatggacttgacaatccactgctgaatggtgaaccattccatgggacctggtttttacatccagaagtcactctcatgttgccatttggagccaaatgtggtgatcatccatggattgcaagaagctag

SEQ ID NO: 25 - DNA, 1128 bp, Selaginella moellendorffii
atggaagatgttttggctgaaaaattgtcaagagtttgcaagttcgatttgccattcatcccttgtagcattccctttgaatgccatcctgattttactaggatatccaaagatactgatgcatgggctcttagaatgttgagtatcactgatccatatgagagaaagaaagctcttcagggaagacatagcttgtatagcccaatgattattccaagaggggagagcagcaaagcggagctttcatcaaagcatacatggactatgtttgttttagatgacattgccgagaattttagtgagcaagagggaaaaaaagctattgatattcttcttgaagttgctgagggaagctatgtcttaagcgaaaaagagaaggagaagcatcctagccacgccatgtttgaggaagtgatgtcgagctttcggagtctcatggatcccccactgtttgctcgctacatgaattgcttgagaaactatctggattctgtggtggaggaggcctctttgcgaattgccaaatctattccaagcctcgagaagtaccggctgctccggagagagacaagctttatggaagcagacggaggcattatgtgtgagttttgcatggatctcaagttgcataagagtgtggtggaatccccagacttcgtagcctttgtcaaggcagtgattgatcacgtcgttcttgtcaatgatctcctttccttccgacacgagctgaaaatcaagtgcttccacaactatctctgtgtcatcttttgccacagcccggataatacaagctttcaagagactgtggacaaggtatgcgaaatgatccaggaggccgaagccgagatcttgcaactccaacagaagctgattaagctgggcgaggaaactggggacaaagatctggtggagtatgcaacatggtacccttgtgtggcatctggaaatcttcgctggtcatacgtcacaggacgctaccatggacttgacaatccgctgctgaatggtgaaccattccaaggaacctggtttctacatccagaagccaccctcatcttgccattgggatccaaatgcggcaatcatccatttatcatgattaagggtcatcatcaccatcaccattga

SEQ ID NO: 26 - DNA, 1104 bp, Selaginella moellendorffii
atggaagatgttttggctgaaaaattgtcaagagtttgcaagttcgatttgccattcatcccttgtagcattccctttgaatgccatcctgattttactaggatatccaaagatactgatgcatgggctcttagaatgttgagtatcactgatccatatgagagaaagaaagctcttcagggaagacatagcttgtatagcccaatgattattccaagaggggagagcagcaaagcggagctttcatcaaagcatacatggactatgtttgttttagatgacattgccgagaattttagtgagcaagagggaaaaaaagctattgatattcttcttgaagttgctgagggaagctatgtcttaagcgaaaaagagaaggagaagcatcctagccacgccatgtttgaggaagtgatgtcgagctttcggagtctcatggatcccccactgtttgctcgctacatgaattgcttgagaaactatctggattctgtggtggaggaggcctctttgcgaattgccaaatctattccaagcctcgagaagtaccggctgctccggagagagacaagctttatggaagcagacggaggcattatgtgtgagttttgcatggatctcaagttgcataagagtgtggtggaatccccagacttcgtagcctttgtcaaggcagtgattgatcacgtcgttcttgtcaatgatctcctttccttccgacacgagctgaaaatcaagtgcttccacaactatctctgtgtcatcttttgccacagcccggataatacaagctttcaagagactgtggacaaggtatgcgaaatgatccaggaggccgaagccgagatcttgcaactccaacagaagctgattaagctgggcgaggaaactggggacaaagatctggtggagtatgcaacatggtacccttgtgtggcatctggaaatcttcgctggtcatacgtcacaggacgctaccatggacttgacaatccgctgctgaatggtgaaccattccaaggaacctggtttctacatccagaagccaccctcatcttgccattgggatccaaatgcggcaatcatccatttatcacgatttga

SEQ ID NO: 27 - DNA, 1062 bp, Selaginella moellendorffii
atggagtttctcttgggaaagattgtccctcgtttcgagttgcctcttcttccaaacaacatcccctgtgcttgccacccggattcctcctctcttagccaggaactcgatgaatggttcatttccaagttaggcatcactgacgagagcgcccagaagaagatcgtccagtcgagaatcatgatctttgcttgcttgatgcatcccaatggcgagagggacagagtcctcctggcagggaagcatttgtgggtgtgcttcttggtggatgacatcctcgagtcaagcaccagggaagcctatggcagcctcaaatccatcgtctggagcattgccaccactggaatctacaaagcatccaatgaggagcatgatcattgtctcgtgctgctgctctaccaggaagttttggcggaactccgcaagaaaatgcccagttctttattcactcgctattgcaagatcctctcaagctacctggatggcgtcgaggaggaggtcaaacaccaggtgaagaacacgatcccgagcagcgaggagtatcggctccttcgccgccgcactggattcatggaggtgatggcgtgcatcatgaccgagttctgcgttggaatcaagctcgaggagtcggttgtaaacttgggagagatccgtaagctcgtcaaggtcatggacgaccacattgtcatggtgaacgacctcctgtcacttcgcaaggagtattacagcagcaccatttgccataactgggtgtttgttttgcttgcggatggctgtggcacttttcaggagagtgtggatcatgtttgcgagatgattaagcaggaggagggttcgattctggatttgcagcagaaacttattatcaaggcaaaggtggacaaaaatccggagcttctcaaatttgcatgtaatgttccaatggcagttgctggtcatctaaagtggtctttcattacggctcgttaccatgggtgtgacaatgctttgctcaatggtgagttgtttcatggaacttggctcatggatcccaatcaaacaataatccagaaaaacatatag

SEQ ID NO: 28 - DNA, 1047 bp, Selaginella moellendorffii
atggctgtttcatccattgcgagcattttcgcagcagagaaaagctattccattccaccagtgtgtcaactccttgtctctccagtgctgaatcctctgtacgatgcaaaggccgagtctcagatcgatgcgtggtgcgcggagtttctgaagttgcaacctggaagcgagaaagctgtgtttgttcaagaaagcaggcttggattgctcgcggcttatgtttacccgaccattccatacgagaagattgttccggttggaaagttcttcgcttcgttctttcttgcagatgacattctggatagcccggagatctcctcgtcggacatgaggaatgtggcaatcgcatacaagatggttttaaagggaagatatgacgaggccacgcttccagtgaaaaatccggagctgctgaggcaaatgaagatgttatctgaggtcttggaagaattgtccctccatgtagtggatgaatcaggccgattcgtggatgctatgaccagggtgctcgacatgtttgagattgaatcgagctggcttcgcaagcaaatcattcccaacctggatacgtacctctggctgagagagatcacatctggtgtggctccttgctttgctctgattgatggtttactgcaacttaggctggaggagcgtggcgtgctggatcatcctctcatacgcaaggttgaggagattgggacgcaccacattgcgctccacaatgacttgatgtcgctaaggaaggagtgggcgagtggaaactacctcaacgccgtgcccattctcgccagcaatcgtaagtgtggcttgaacgaggcaatcggcaaggttgcgagcatggtggaggatttggagaaggatttcgcccagacaaagcatgagatcatttcaagtgggcttgccatgaagcaaggagtcatggactatgtgaacggcatagaggtgtggatggccggaaacgtagaatggggatggacgaccgctagataccatggaattgggtggatccctcctccagaaaaatcagggaccttccaactctag

SEQ ID NO: 29 - DNA, 1062 bp, Selaginella moellendorffii
atggagtgtctcatggcaaagcttgtccctcgccttgagttgcctcttcttccaaataacatcccctctgcttgccactgggattcttcttctctcagccaagagctcgatcaatggctcatctccaagctcggtatcaccgacgagagtgccaagaggaagattgtccagtcgagagtcatgctcttagcttgtttgatgcatcccaatggcgagagggacagagtcctcttggcagggaagcatttgtgggtgtacttcctggtggatgacatcctcgagtcaagcagccgggaaggttatggcgccctcaaatccatcgtctggagcattgccaccactggaatctacaaagcatctgaggagcatgatcatcatgacctcgtgctgcttctcttggtggaagtcatggtggaactccgcaaggaaatgcccacttctttattcgctcgctactgcaagatcctctcaatctatctggatagcgtccaggaggaggtcaagcaccaaatcaataacacgatcccgagcagcgaggagtaccggcttctccgccgccgcactggattcatggaggtgatggcctgcatcatgactgaattttgcgtgggaatcaacctcgaggaattggttgtaaacttgggagagatccgtgagctcgtcaagatcatggacgaccacattgtcacggtgaacgacctcttgtcactgcgcaaggagtattacaatggcaccatttaccacaactgggtaattgttttgcttgcccatgattgtgcaacttttcagaagagtgtggatcgcgtttgtgagatgatcaagcaggaggaggactcgattctagatttgcagaagaaacttatcatcaaggcaaaggtggacaagaacccagagcttctcaaatttgcatttaatgttccaatggctgtggctggtcatctaaagtgggcttttattactgctcgttaccatggttgtgacaacgctttgctcgatggtgagttgtttcatggaacttggatcatggaccccaatcaaacggtaatcgtgaaaaacatgtag

SEQ ID NO: 30 - DNA, 959 bp, Selaginella moellendorffii
atggctccctacgatttcgttccaaatgtgcagtgttcgttccctgtgaagtgccaccctctgtattctttcattcgtccacgcttggaagattgggctgcaactttggagcctgggcatggtgaagggaaccccaaaagtaggaaatgccttgttgctgagaaaagagttctagccacttgcatgttgatccctgttgctgatgatgccagaattgagaatatgtgcaagcttgcctgttggtgcttccatgttgatgatatccttgacgacttgcagggcctgggagctgactcgggaggtgccaagaggcttgttgatagctaccttggcataatccgtgcgtggcagatatggaatttccacggttctgtgatatgtggaatgatctgcgtgcagatatgccactcaagcagtaccagcgatttgccaacagagtgtccgagctgttggaggcaagcgtgaatcaggtgaagctaaggaatctgaaaacggtgatgggcttggaggagctgctggctcaccgtcgcgtgttagttggtgtatttgttatggaaactctaatggagtatggcatgggattcgaactccaggacgacgccatttcaaatcaggacctccaagaggctgaaagtctggttgcagaccacgtaagttgtgtcaacgacttgttttgcttcctggtggacagtgcaactggcgatactcagttcaatatcgtccacacgatcatgtgcggcaattcggattcaaagggcttttctttcgagtatgcagccgacaaagttcacaaactggtgcagagtatcgagcatcgattcaagaagctgtgtgagaatatcagaagatcaagctgctacaatggtgcaatggaggcttacctggaaggcttgtctcatattatatccggcaaccttgagtggcaccggcagacaggacgatacaaactggtatcttga

SEQ ID NO: 31 - DNA, 630 bp, Selaginella moellendorffii
atggggatgcttaacgatgtctacactgatctaaagggtttcatgaagcctggacataagacccggttttcgaattccatgattgatgtgctcgacatgtttgaggtggaatcaagttggcttcacaagaaactggttcccaactttgagatttatatgtggatgagggaggtaacggctggggttatcccttgcatggtggcaatagacttccttaataattttgggctggaagaggaaggaatgctagacgatctccatattcaaacgctggaagtgattgcaaatcgccattcgttcctagccaacgacatggtctccttcaaaaaggaatgggcttgtgaacagtacctcaattctgtggcactggttggttacagtagcaactgtggcttaaacgaggcaatggagaaagttgcagaaatggttcaggatttggagaaagagtttgctgacatcaaacaaaaggttctgtcaaacaaggacttgaacaagggaaatgtcatggggtatgtgcaaggcttggagtatttcatggctggaaatatagagtttagctggctctctgcgagatatcatggggtgggatgggtttcaccagctgagaaatatggtaccttggagttctag

SEQ ID NO: 32 - DNA, 1119 bp, Selaginella moellendorffii
atggcaagtccgtgtttacagaagctaccagctgtagaacatctctttgctctcacgaggttcgagcttccggagataccatgctctctatctttccaaaggcatcccgagtatatgtcaatcactaaggaagcaaacgagtgggcattcaaatgcatgaggagggatttcagtccggaggagaagaaatgcctggtccagtggaaagttccaatgtttacgtgcctctccacgcctcacgctccgaaagcgaacatggtggcgtcggccaagtttgcctggctcactgccttcctggacgatccgtttgatgacaacgaggttgctgggggagctctcgcgacatcgtatctcgacactgttcttagcctctgctacggaaccgcctcgctcgcggaaattccggacattcttgcctatcgagcgtgccacgatctgatgaaggatttgaggtctctgttgaagccgaagctgtttaagcgcacggtttccactgttgagggctgggcgagaagcatctcgagtgacgacttgacgcaggactacgagctctaccggaggaagaacgtcttcattctgccactcatctacgcaatgggtgcttcgtttgacgatgaggatgtcgagtccctggattacatcagggctcagaacgctatgctcgatcatatgtggatggtgaacgacgtcttctcgtttcccaaggagttttacaagaagaagtttaacaatcttccggcggtgctgctgctcaccgatccgagcgtgcagacgtttcaggacgccgtgaacactacgtgcaggatgatccaggacaaggaagacgaattcatctattaccgtgatatccttgccacgaatgcgtcccggaatgggaagaaagatttcctgaagttcctggatgttctctcctgcgcaatcccagcgaatctggtgttccattatgcgagcagccgctaccatggcatggataaccccctactgggtggacccacgtttagtgggacctggattctggatccaaagcgcaccatcatcttgtcggacccgaaaaggtggaacgtggtggcaagttcaaacaaactcaaccagatccaaaatttatcaaacttgatatga

SEQ ID NO: 33 - DNA, 534 bp, Selaginella moellendorffii
atgggtgctttatttgacgatgaggatgttgaatccctggattacatcagtgctcagaacgctatgctcgatcatatgtggatggtgaacgacgtcttctcgtttctcaaggagttttacaagaataagtttaacaatcttccggcggtgctgctcaccgatcagagcgtgcagacgtttcaggacgccgtgaacactacgtggaggatgatccaggacaaggaagacgaattcatcttttaccgcgatatccttgccgcgaatgcgtcccggaatgggaagaaagatttcctgaagttcctggatgttctctcctgcgcaatcccagcgaatctggtgtatgcgagcagccactaccatggcgtggataacctactgagtggaggcacgtttcgtgggacttggattctggatccaaagcgcaccatcatcgtgtcggacccgaaaagttgcaatgtggtggcaactacggacgaagtgaaaatcaatgtttcatatgcatggctatttgtcattctaattcttgcaaactga

SEQ ID NO: 34 - DNA, 1521 bp, Selaginella moellendorffii
atgccaggggagtacagcttctacaacttccttgacatgggagtcgcgccttacggcgattactggaagaacatgcggaagctgtgcgccacaggcaccattcccagccggagagagaagatcggtccatacttgttggacagtgcaaggagagagagatggggtttcctcccaaagaggtccgatcaatggaagagacccactgggtgaaaactgtcaagttgagacggcggtgccctccacctggaattccccagacgagaacatagatgaagagcaatcgcgcatgctagaaaccccatgttgaattacgccacaaacgacgtcttgatatcattttattcattcattcattatattctctcagaacaaaagagagtagtccttttttcagtgaataattgaccggtctattctttgccaggtgtgatttgacaaccaccggttcaacctgcacaatcaaagtcagtccaagcaagacagcatgatcaagtgaaacttagcaatataaattgaagagctctgaaatatctcgaaaccatctctaaagaaatggcaagtccgtgtttacagaagctaccagcggtagaacatctctttgctctcacgagtttcaagcttccggagataccatgctctctatctttccaaaggcatccggactatatgtcaatcaccaaggaagcaaacgagtgggcattcaaatgcatgaggagggatttcagtccggaggagaagaaatgcctggtccagtggaaagttccaatgtttacgtgcctctccacgcctcacgctccgaaagcgaacatgttggcatcggccaagtttgcctggctcactgccttcctggacgatccgtttgatgacaacgaggttgctgggggagctctcgcgacatcgtatctcgacactcttcttagcctctgctatggaaccgcatcgctcgcggaagttccggacattcttgcctatcgagcgtgccacgatctgatggaagatttgaggtctctgtttaagccggagctgttcaggcgcacggtttccactgttgagggctgggcgagaagcattttgagtgacgacttgacgcacgagtacgagctctaccggaggactaacgtcttcattttgccactcatctacgcaatgggtgcttcgtttgacgatgaggatgtcgagtccctggattacatcagggctcagaacgctatgctcgatcatatgtggatggtgaatgacgtcttctcgtttcccaaggagttttacaagaagaagtttaacaatcttccggccgtgctgctgctcaccgatccgagcgtgcagacgtttcaggacgccgtgaacactacgtgcaggatgatccaggacaaggaagacgaattcatctattaccgcgatatccttgccagcgtcccggaatgggaagaaagctttcctgaagttcctggatgttctctcctgcacaatcccagcgaatctggtgttccattatgcgagcagccgctaccatggcatggataa

SEQ ID NO: 35 - DNA, 1104 bp, Selaginella moellendorffii
atggggagcctgtgtttgcagaagctatcagcggtagaacgtctcttcgctctcgagagtttcgagcttccggaggtaccatgctctctctctttccacaggcaccccgagtacaagtcaatcaccagggaagcaaacgagtgggcattcaaatgcacgaggagggatttgagtccggaggagaagaaatccctgctccagtggaaggttccaatggtgacatgcctttccacggctcacgctccgaaagagaatatggtggcgtcggccaagtttgcttgggccattgccttcctggacgacccgattgatgacaacgaggtcgccgcaacgtcgtatctcgacactgttcttagcctctgcaatggaaccgcatcgcttgcggaagttccagacattgttgcgtatcgagcttgccacgatctgatgaaggatttgaggtctctgttgcagccggagctcttcaagcgcacagtttccactgtcgagggttgggcgagaagcatctcgagtgacgacttgaagcaggactacaagctctacaggaggaacaacatcttcattctgccactgttctacacactcattggcgcttcctttgaagatgaggatgtcgagtccccggatttcgtcagtgctcagaacgctatgcttgatcatatatggatggtgaacgatatcttctcgtttcgcaatgagttctacaagaagaagttgaacaacctgccggctgtgctgctgctcaccgatccgagcgtgcagacgtttcaggaagcagtgaacgctacatgcaggatgatccaggacaaggaagaagaattcatctattaccgcaacatccttgccgcgaacgcgtcccggaatgggaaggacttcttgaagttcctggatgttctctcctgcgcaatcccggcgaatctggcgttccactatgcgagcagccgctaccacggcatggataaccctcttctggccgggggcacgtttcatgggacctggattctggatccaaagcgcaccatcatcgtttcggacccgaacagaagtaacggagcggcatcaaacaaactcaaccatatccaagatttatcaaagttgatatga

SEQ ID NO: 36 - DNA, 1119 bp, Selaginella moellendorffii
atggccgtatataagcagggtagcggattcaaaaccgaggcatccgtaattttgggtgtcacccatttcgagctcccattgcttcccaacaacattgcattttattgccacccggaattccaatcaatcagcctccaaatcgacgagtggttccttgacaagatgagaatcgccgacgagacttccaagaagaaggtgctggagtccaggatcggtttgtacgcctgtatgatgcatccccatgctgagagagagaagattgtgctggccgggaaacatctctgggccgtcttcctccttgacgatttgctggaatccagcggcacacaagagatgccgaagctcaacgccaccatttccgaccttgccagtggaaattccaacgaggatgttacaaatcctgtgttggttctctaccgagaagttatggaagagatccgggctggtatggagccaccattgctggatcgctacgtggagtgcctgggagcttcactggaagccgtgaaggatcaagttcaccaccgagccgagaaaagtatccctggagtggaagcttacaagcttgcccgccgtgccactggattcatggaagctgtcggcggtatcatgaccgagttctgtatgggaatccgcctcaacgaaagtcaaatccagtctccagtctttcgagagctcctcaattctgtgtctgatcacgttgttcttgtcaatgatctcttgtccttccggaaagagttctatgaaggtgcttgtcaccacaactggatctcagttctcctgcagcacagccccagcgggacgaggttccaggatgtcattgatcagctctgcgagatgatccaagaagaagagctctcaatcctggcattgcagaggaagatttccagtaaagaaaatagcgactcggagctgatgaagttcgcaagggagttcccaatggttgcttccgggagcctagtgtggtcgtatgtcactggccgctaccatggctatggtaatccgctgctgactggggagattttcagcggaacttggctgctccatcccatggccaccgtcgtcttgccaaagtctacagtcttttcattaaaccatttggtatattctcatgtttga

SEQ ID NO: 37 - DNA, 1116 bp, Selaginella moellendorffii
atggcaagtccgtgtttacagaagctaccagcggtagaacatctctttgctctcacgccggagatacctttccaaaggcatcccgagtatatgtcaatcaccaaggaagcaaacgagtgggcattcaaatgcatgaggagggatttcagtccggaggagaagaaatgcctggtccagtggaaagttccaatgtttacgtgcctctccacgcctcacgctccaaaagccaacatggtggcgtcggccaagtttgcctggctcactgccttcctgaacgatccgtttgatgacaacgaggttgctgcgggagctctcgcgacatcgtatctcgacactgttcttagcctctgctatggaaccgcatcgctcgcggaagttccggacattcttgcctatcgagcgtgccacgatctgatggaggatttgaggtctctgttgaagccggagctgttcaagcgcacggtttccactgttgagggctgggcgagaagcatctcgagtgacgacttaacgcaggactacgagctctaccggaggaagaacgtcttcattctgccactcatctacgcaatgggtgcttcgtttgacgatgaggatgttgagtccctggattacatcagggctcagaacgctatgctcgatcatatgtggatggtgaacgacgtcttctcgtttcccaaggagttttacaagaagaagtttaacaatcttccggcggtgctgctgctcaccgatccgagcgtgcagacgtttcaggacgccgtgaacactacgtgcaggatgatccaggacaaggaagacgaattcatctattaccgtgatatccttgccacgaatgcgtcccggaatgggaagaaagatttcctgaagttcctggatgttctctcctgcacaatcccagcgaatctggtgttccattatgcgagcagctgctaccatggcatggataaccccctactgggtggaggcacgtttcgtgggacttggattctggatccaaagcgcaccatcatcgtgtcggacccgaaaagccaagccattcctcacgcggtgcacatatggaagtcagcagtattctatgctcagtcttatttcatccagagcttagaagactag

SEQ ID NO: 38 - DNA, 1119 bp, Selaginella moellendorffii
atggcaagtctgtgtttacagaagctaccagcggtagaacatctctttgctctcacgagattcgagcttccggagataccatgctctctatctttccaaaggcatcccgagtatacgtcaatcaccaaggaagcgaacgagtgggcattcaaatgcatgaggagggatttcagtccggaggagaagaaatgcctggtccagtggaaagttccaatgtttacgtgcctctccacgcctcacgctccgaaagcaaacatggtggcgtcggccaagtttgcctggctcactgccttcctggacgatccgtttgatgacaacgaggttgctgggggagctctcgcgacatcgtatctcaacactgttcttagcctctgctatggaaccgcatcgctcgcggaagttccggacattcttgcctatcgagcgtgccacgacctgatgaaggatttgaggtctctgttgaagccggagctgttcaagcgcacggtttccactgttgagggctgggcgagaagcattttgagtgacgacttgacgcaggactacgagctctaccggaggaagaacgtcttcattctgccactcatctacgcaatgggtgcttcgtttgacgatgaggatgtcgagtccctggattacatcagggctcagaacgctatgctcgatcatatgtggatggtgaacgacgtcttctcgtttcccaaggagttttacaagaagaagtttaacaatcttccggcggtgctgctgctcaccgatccgagcgtgcagacgtttcaggacgccgtgaacactacgtgcaggatgatccaggacaaggaagacgaattcatctattaccgcgatatccttgccacgaatgcgtcctggaatgggaagaaagatttcctgaagttcctggatgttctctcctgtgcaatcccagcgaatctggtgttccattatgcgagcagccgctaccatggcatggataaccccctactgggtggaggcacgtttcgtgggacttggattctggatccaaaatgcaccatcatcgtgtcggacccgaaaaggtgcaacgtggtggcaagttcaaacaaactcaaccagatccaaaatttatcaaacttgatatga

SEQ ID NO: 39 - DNA, 966 bp, Selaginella moellendorffii
atgccaggggagtacagcttctacaacttccttgacatgggattcgcgccttacggcgattactggaagaacatgcggaagctgtgcgccacaggcaccattcccagccggagagagaagatcggtccatacttgttggacagtgcaaggagagagagatggggtttcctcccaaagaggtgtgatttgacaaccaccggttcaaatattttccctacacaatcaaacctctgctatggaaccgcatcgctcgcggaggttccggatattcttgcctatcgagcgtgccacgatctgatgaaggatttgaggtctctgttgaagacggagctgttcaggcgcacggtttccactgttgagggctgggcgagaagcattttgagtgacgacttgacgcaggactacgagctctaccggaggaagaacgtcttcattctgccactcatctatgcaatgggtgcttcgtttgacgatgaggatgtcgagtccctggattacatcagggctcagaacgctatgctcgatcatatgtggatggtgaacgacgtcttctcgtttcccaaggagttttacaagaagaagtttaacaatcttccagcggtgctgctgctcaccgatccgagcgtgcagacgtttcaggacgccgtgaacactacgtgcaggatgatccaggacaaggaagacgaattcatctattactgcgatatccttgccagcgtcccggaatgggaagaaagctttcctgaagttcctggatgttctctcctgcgcaatccagcgaatctggtgttccattatgcgagcagccgctaccacatggataaccccctgggtggaggcacgttttgtgggacttggattctggatccaaagcgcaccatcatcatgtcggacccgagaaggtgcaacgtggtggcaagttcaaacaaactcaaccagatccaaaatttatcaaacttgatatga

SEQ ID NO: 40 - DNA, 908 bp, Selaginella moellendorffii
atggctccagctctagagaagatctatgctgttgctaggcctcaagaatttccatctcccaaagatacccagcttgattcctccagcgcttatgcatccagcaacaagttcatcacccgcggataaaaaggctttaaaagattggaagatcccactgtttggaactccggtagagtcgattgggtccaggaaaaatgctctcacatgctcataatactgcttgtgggctacattactggacgacttggttgacgggggtttgcttgagactgctagcattcttcaagattactactccataatcctgaatcatctacacaatcctgaagtcaagatgcctggaggcatcggacaatcaccttccagttcgagtttacagggccactgaagagagataagatcatccatgcttcctccagtgtatgcttattttgtagcacagtttgagaggtatgcactcagcagaatggcaagcaggccctgtcaagcagtctatcgagtggagaaggtttgaagtgttattagagcctatcttcagcttcatagagatggcgtttgaagtcgcatagagatggcgttggaatcagaggactatctaattctgcgagatcccaggattgactatgtatctatgcacaaccatattctcccgttcgtgaaggtgttcgtaactgctgaatcttccagtgttgctgcttctttcggatccacattctgatcgctcaggcaagaggtgaaggtaaacacgagtttgtgaagtatcttgagtgtcatcctagtgttctctccaacacgctttactatcactaccctctgcccggtaccatccagcattcatcactggtgagaagtttgacgggaattggtgtttgggcactgttataaaccatagaagaactggccggtga

SEQ ID NO: 41 - DNA, 1041 bp, Selaginella moellendorffii
atggcatttgttgtggagaagattcccgccatggaacaccacctggggctaaagaggttctatttgccgcccattcgctgctccatcccctccagcgcctgggatcccgaccacaagctggttgccaagctcgcgaacgagtgggcattcccattcatcaatcccagcatgagcgatgcccaaaagctctccctggagcgcatgcgaatcccgctctacatgagcatgctcgtgccgtgcggatccaccgagagcgccgtcctctgcggcaagttcgcgtggtttggaaccatgctggatgatctcctcgaggacgagtcccccggcggcgccccccgggaggagttcctggagactttccagggcatcctccatgggacacacccacatcgcgatccagtccatccatcgctcgagttctgcgcggacctcattccgcgcctgcgatcatccatggctccccgggtgtacgcgcactgggtcgcgcagatggaggcctacgctgcctccatggaccggagcgtcctttctctagcacaatcggcgtcgacggtcgagagctacttggcgcgcaggaggctcgattgcttcctcctcccctgcttcccattcatcgagatgtcgctggagattgcgctcccagacagcgatttggagtcgcgggactacctggcgctccagaacgccatcaacgatcacgtcctccttgtcaacgacgttatctccttccccgcggagctgcgcgccaaaaagccactgagaagcatcgcgtccttgcagttgctcttggatcccagcgtcaacacgttccaggactcggtggacaggacctgtgcaatgatccaggagaaggaacgcgaggtgacgcattactacgacgttgtgatgaggaacgctgtggcttctggcaatgccgagcttgtgagctaccttcagatcctcaagatgtgcgttccaaacaacctcaagttccacttcattagttctcgttatggagtgaatgatgccgagtctggtcatggaatttggattgttctgtag

SEQ ID NO: 42 - DNA, 1326 bp, Selaginella moellendorffii
atgggctacgtgggagtaaacatggaagttctcgtggattgccgcaacaccgtctttgcgaaaggccttacatcactggaggagctctggtggtggtgttttggacgccatgggttcttaactcaatgtactctgaaacggagattgatattgtcaaagggaacctgcagacaattgagtattaccaatagacctttctccttgtatatttcttggcgggtgctacccaggcattatatagcttacacagccctggagaaacatagaagaagatccatcatgggagcctcctctatccttagcatttttgagggcgccaaaagcttttacataccaccgcacagctcttatcatgttgatttgaatcctgcttatgatgcaaagttggatgctgaaattgacaaatggtgtatggattttttgaacctgcatgatctcactgatcacaagactcagtttgccattcagagcaagcttggaaaacttgcaggctttgcataccaagccatttcttcagagagattgagtcccatcgcaaaattcttttgctggttgtttcttgcagatgattttatggacgatccttctgtccctgtctctgacctgaagaatgccacacttgcatacaagctcatcttcaaaaacgactatgatcaagccataactctggtggaaagtaaaggccttctgcggcaaatggggatgcttaacgatgtctacactgatctaaagggtttcatgaatcctggacataagacccggttttcgaaatccatgattgatgtgctcgacatgtttgaggtggaatcaagttggcttcacaagacactggttcccaactttgagatttatatgtggatgagggaggtaacagctggggttatcccttgcatggtggcaatggacttccttaataattttgggctggaagaggaaggagtgctagacgatccccatattcaaacgctggaagtgattgcaaatcgccattcgttcctagccaatgacatggtctccttcaaaaaggaatgggcttgtgaacagtacctcaattctgtggcactggttggttacagtagcaactgtggcttaaacgaggcaatggagaaagttgcacaaatggttcaggatttggagaaagagtttgctgacatcaaacaaaaggttctctcaaacaaggacttgaacaagggaaatgtcatggggtatgtgcaaagcttggagtatttcatggctgcaaatatagagtttagctggatctctgcgagatatcatggggtgggatgggtttcaccagctgagaaatatggtacctttgagttctag

SEQ ID NO: 43 - DNA, 1239 bp, Selaginella moellendorffii
atgaggaggggcagcgcctacaccaagcaagagctcctcgcgcatcacatgggctacatgggagtaaacatggaagttctcatggattgccgcaacaccctctttgcgaaagaccttacatcactggaggagctctggcggtggtgttttggacgctgtgtttggtgtacgccacccaggaacatgcggtggacgagactttggtgcttctttgaggagtttacatcgtccgatatcaatctccatttcagagtacattgggttgagggtcaagcagaattagacattggaagtagaaaggtggatgctgaaattgacaaatggtgtatggattttttgaacttgcacgatctcactgatcacaagactcagtttgccattcagagcaagctgggaaaacttgcaggcttggcataccaggccatttcttcagagagactccgtcccatggcaaaattcttgtgctggttgtttcttgcagatgattttatggaagattcttctgtccctgtctctgacctgaagaatgccacacttgcatacaagctcatcttcaaaaacaactatgatcaagccataactctggtggaaagtaaagaccttctgcggcaaatggggatgcttaacgatgtctacactgatctaaagggtttcatgaagcctggacataagacccggttttcgaattccatgattgatgtgctcgacatgtttgaggtggaatcaacttggcttcacaagaaactggttcccaactttgagatttatatgtggatgagggatgtaacggctggggttatcccttgcatggtggcaatagacttccttaataattttgggctggaagatgaagtgctagagcatcccaacattcaaaggctggaagtgattgcaaatcgccacacgtacctagccaatgacatgctctccttcaaaaaggaatgggcttgcgacatgtacctcaattctgtggcactggttggttacagtagcaactgtggcttaaatgaggcaatggaaaaagttgcagaaatggttcaggatttggagaaagagtttgctgacaccaaacaaaaggttctctcaaacaaagacttgaacaagggaaatgtcatggggtatgtgcaaggcttggagtatttcatggctggaaatctagagtttgcctggctctctgcgagatatcatggggtgggatgggtttcaccagccgagaaatatggtacctttgagttctag

SEQ ID NO: 44 - DNA, 1047 bp, Selaginella moellendorffii
atgggaccctcctctatccttagcatttttgagggcgccaaaagcttttacataccaccgcacagctcttatcatgttgatttgaatcctgcaaaattggatgctgaaattgacaaatggtgtatggattttttgaacctgcacgatctcactgatcacaagactcagtttgccattcagagcaagctgggaaaacttgcaggcttggcataccaggccatttcttcagagagactgcgtcccatggcaaaattcttgtgctggttgtttcttgcagatgattttatggacaatccctctgtccctgtctctgacctgaagaatgccacacttgcatacaagctcatcttcaaaaacgactatgatcaagccataacgctggtggaaagtaaagaccttctgcggcaaatggggatgcttaacgatgtctacactgatctaaagggtttcatgaatcctggacataggacccggttttcgaaatccatgattgatgtgctcgacatgtttgaggtggaatcaagttggcttcacaagaaactggttcccaactttgagatttataacgtaacggctggggttatcccttgcatggtggcaatagacttccttaataattttgggctggaagatgatgtgctagaccatcccaacattcaaaggctggaagtgattgcaaatcgccacacgtacctagccaatgacatggtctccttcaaaaaggaatgggcttgcgacatgtacctcaattctgtggcactggttggttacagtagcaactgtggcttacacgaggcaatggagaaagttgcacaaatggttcaggatttggagaaagagtttgctgacatcaaacaaaaggttctgtcaaacaaggacttgaacaagggaaatgtcatggggtatgtgcaaggcttggagtattttatggctggaaatataggctctctgcgagatatcatggggtgggatgggtttcaccagctgagaaatatggtaccttggagttctagtttgcttctacttgcattagaagctggtgcttaa

SEQ ID NO: 45 - DNA, 972 bp, Selaginella moellendorffii
atggccgcgccttctatctatcgtccccaaattctggagcagctcctcgcctgcaagagcatctacttgcctcaaatccgctgctcgctgccattgcagtgccacccagactacgcctccgtctccagacaggcgaacgattgggcctttcgcttcctcaagatcaatgccaccaatgccgctgcagagaagaaatgcttcacccagtggaggacgccactctacggcaccttcgttgtgccttggggcgactccaggcacgctctagcggccgccaagtacacctggctcatcaccattctcgacgatgctgtcgacgaggagccttcgcagcggaacgagatcctggaagcttacatgagccttgcctccggaaatttgttggctgctactcggacgaagaggagttcacgaaagagtcttaacgctgtgcatcgccgggaggacttcgttgtcaagccgatgcttaacttcacgcagatgtgcctcggagtgaagctgagagacaaggatctggaaagcgaggagtacctccgggcgatagatgccatgtttgatcacatctggctggtgaacgacatcttttcattcccaaaggagctgaggaagaaaactttcaagaacataatttttctcttgctcttcacggaccacaccgttcgctctgttcagcaggcagtcgataaggcgaatgccatggttcaggaaaaagaacaagaattcatgtattaccacgagatcctgacgaggaaagcgatggaatctggcaaccacgactttctggcgtaccttagagcgattccggcattcatccctggaaacctacgttggcactacctcacagctcggtaccacggtgttgataatccatttgtaacaggagagccattcagtgggacttggttgtttcatgatacgcagactatcatactccccgagtacaaaccaactcatccccatctgcaagtctga

SEQ ID NO: 46 - DNA, 1101 bp, Selaginella moellendorffii
atggccgcgccttctatctatcgtccccaaattcttgagcagctcctcgcctgcaagagcatctacttgcctcaaattcgctgctcgctgccattgcagtgccacccagactacgcctccgtctccagacaggcgaacgattgggcctttcgcttcctcaagatcaatgccaccaatgccgctgccgataagaaatacttcacccagtggaggatgccactctacggcacctttgttgtgccttggggcaactccaggcacgctctagcggccgccaagtacacctggctcatcaccattctcgacgatgctgtcgacgaggagccttcgcagcgggacgagatcctggaagcttacatgagccttgcctccggtcaaagatccatcgcccaagttcccaacaaacccgtgctcgtcgcccaagccgagctcgtcccggatctgcggaagctcatgtcgccgctcctcttccagcggctgctcgtctcgtacaggaaatttgttggctgctactcggccaaagtcgacgaggaggagttcacgaaagagtcttacgctgtgcatcgccgggaggactacgttgtcaagccgatgcttaacttcacgcagatgtgcctgggagtcgagctgagagacaaggatctggaaagcgaggagtacctgcgggcgatagatgccatgtttgatcatatgtggctggtgaacgacatcttttcattcccaaaggagctgaggaagaaaactttcaagaacataatttttctcttgctcttcacggaccacaccgttcgctctgttcaacaggcagttgataaggcgaacgccatgattcaggaaaaagaacaagaattcatgtattaccacgagatcctgacgaggaaagcgatggaatctggcaaccacgactttctggcgtaccttagagcgattccggctttcatccctggaaatctacgttggcactacctcgcagctcggtaccacggtgttgataatccatttgtaacaggagagccattcagtgggacttggttgtttcatgatacacagactatcatactccccgagtacaaaccaactcatccccatctgcaagtttaa

SEQ ID NO: 47 - DNA, 471 bp, Selaginella moellendorffii
atggggatgcttaacgatgtctacactgatctaaagggtttcatgaatcctggacataagacccagttttcgaattccatgattgatgtgctcgacatgtttgaggtggaatcaagttggcttcacaagaaactggttcccaactttgagatttatatgtggatgagggaggaatgggcttgtgaacagtacctcaactctgtggcactggttggttacagtagcaactgtggcttaaacaaggcaatggagaaagttgcagaaatggttcaggatttggagaaagagtttgctgacatcaaacaaaaggttctgtcaaacaaggacttgaacaagggaaatgtcatggggtatgtgcaaagcttggagtatttcatggctgcaaatatagagtttagctggatctctgcgagatatcatggggtgggatgggtttcaccagctgagaaatatggtaccttggagttctag

SEQ ID NO: 48 - protein, 349 aa, Selaginella moellendorffii
MAILSIVSIFAAEKSYSIPPASNKLLASPALNPLYDAKADAEINVWCDEFLKLQPGSEKSVFIRESRLGLLAAYAYPSISYEKIVPVAKFIAWLFLADDILDNPEISSSDMRNVATAYKMVFKGRFDEAALLVKNQELLRQVKMLSEVLKELSLHLVDKSGRFMNSMTKVLDMFEIESNWLHKQIVPNLDTYMWLREITSGVAPCFAMLDGLLQLGLEERGVLDHPLIRKVEEIGTHHIALHNDLISFRKEWAKGNYLNAVPILASIHKCGLNEAIAMLASMVEDLEKEFIGTKQEIISSGLARKQGVMDYVNGVEVWMATNAEWGWLSARYHGIGWIPPPEKSGTFQL

SEQ ID NO: 49 - protein, 358 aa, Selaginella moellendorffii
MALALDKIYAIEKLLGLKNFHLPKIPCSIPSVPCHPDSIYASNKAHEWAYKFMDPKMTAADRKALEDWKIPMFATLVVPFGSKRNAVICSKYSMFALLVDDSVDEGFVESTILQDYYSTILNHLHNPNFKIQASDDHLPLRVYRATEELVTEIRSSMLPPVYAHFVAQFERYALSRMASRPKFQSVKQYIEWRRFDVFLEPIFSFIEMALEVAVPDTELESEDYLILRDAGIDYISMYNDVLSFAKEFACNKLLNLPVLLLLSDPEVESFQNAVDKSCKMIVDKEQEFVYYHNILITQARGEGKHAFVKYLECLPTVLSNTLYYHYSSARYHPAFITGEKFDANWCLDTVINHRRTGR

SEQ ID NO: 50 - protein, 366 aa, Selaginella moellendorffii
MAAPSIYRPQILEQLLACKSIYLPQIRCSLPLQCHPDYASVSKQANDWAFRFLKINATNAAADKKYFTQWRMPLYGTFVVPWGDSRHALAAAKYTWLITILDDAVDEEPSQRDEILEAYMSLASGQRSIAQVPNKPVLVAQAELIPDLQKLMSPLLFQRLLVSYRKFVGCYSAKVDEEEFTKESYAVHRREDYVVKPMLNFTQMCLGVELRDKDLESEEYLRAIDAMFDHMWLVNDIFSFPKELRKKTFKNIIFLLLFTDHTVRSVQQAVDKANAMIQEKEQEFMYYHEILTRKAMESGNHDFLAYLRAIPAFIPGNLRWHYLTARYHGVDNPFVTGEPFSGTWLFHDTQTIILPEYKPTHPHLQV

SEQ ID NO: 51 - protein, 366 aa, Selaginella moellendorffii
MAAPSIYRPQILEQLLACKSIYLPQIRCSLPLQCHPDYASVSKQANDWAFRFLKINATNAAADKKYFTQWRMPLYGTFVVPWGDSRHALAAAKYTWLITILDDAVDEEPSQRDEILEAYMSLASGQRSIAQVPNKPVLVAQAELIPDLQKLMSPLLFQRLLVSYRKFVGCYSAKVDEEEFTKESYAVHRREDYVVKPMLNFTQMCLGVELRDKDLESEEYLRAIDAMFDHMWLVNDIFSFPKELRKKTFKNIIFLLLFTDHTVRSVQQAVDKANAMIQEKEQEFMYYHEILTRKAMESGNHDFLAYLRAIPAFIPGNLRWHYLAARYHGVDNPFVTGEPSSGTWLFHDTQTIILPEYKPTHPHLQV

SEQ ID NO: 52 - protein, 258 aa, Selaginella moellendorffii
MAPYDFVPNVQCSFPVKCHPLYSFIRPGLEDWAATLEPGHGEGNPKGLGADLGGAKRLVDSYLGIIHAPEPVADMEFPRFCDMWNDLRADMPLKQYQRFANRVSELLKASVNQVRLRNLKTVMGLEELLAHRRMLVGVFVMETLMEYGMGFELQDDAISNQDLQEAESLVADHCNWRYSVQYRPCGNSDSKGFSFEYAADKVQKLVQSIEHRFKKLCENIRRSSCYNGAMEAYLEGLSHIISGNLEWHRQTGRYKLVS

SEQ ID NO: 53 - protein, 423 aa, Selaginella moellendorffii
MAASVNGVLPELSTLSKFELRPLPCAFPFECHPNHASLTREVDEWAIRSLQARGSMPKRQMIIESKISAAACMTIPRGRDDRKMVLAGKHLWALFLLDDALESCRSQEAARVLARRAMEVARGDQLEGMIQEERELEEAKGVARKFAIQEEEGDRYNDQSRGILANIAIQEDPGLIDLATRGMATKIAITEEDQGRDSRWALGLFREVVAELRRSMPLPMFDRYLRYLDRYLEAVIQEVGYQIAGHIPREDEYRELRRGTSFTEGTSAIFGELCMGLELHESVTSSRDFIEFVALVADHIALTNDVLSFRKDFYAGVAHNWLVVLLRHSHRGTGFQSALDSVYGMIRDSECRILGLQSRIEAQALKSGDGHLLSFAQAFPLCLAGNRRWSSITARYHGIGNPLITGVEFHGTWLLHPDVTIVI

SEQ ID NO: 54 - protein, 346 aa, Selaginella moellendorffii
MALAVEKIPAMEHLLGLKRFYLRPIRCSIPSSAWHPDHKLVAKLANEWAFPFINPSMSDAQKLSLERMRIPLYMSMLVPCGSTESAVLCGKFAWFGTMLDDLLEDESPGGAPREEFLETFQGILHGTHPHRDPVHPSLEFCADLIPRLRSSMAPRVYAHWVAQMEAYAASMDRSVLSLAQSASTVESYLARRRLDCFLLPCFPFIEMSLEIALPDSDLESRDYLALQNAINDHVLLVNDVISFPAELRAKKPLRSIASLQLLLDPSVNTFQDSVDRTCAMIQEKEREVTHYYDVVMRNAVASGNAELVSYLQILKMCVPNNLKVHFISSRYGVNDGESGHGIWIVL

SEQ ID NO: 55 - protein, 159 aa, Selaginella moellendorffii
MSSFRSLMDPPLFARYMICLKTFLDSLVEEASLRSAKSIPSLEKYQLLRRGTVFVEGAGDFVAFVNAVADHVLFSFRHEMKIKCFHNYLCVIFCHSPNNASFQEAVDKVCKMIQETEAKILQLQKKLMKMGEETGNKDLVDYATWYPCFTSGHLRWPWA

SEQ ID NO: 56 - protein, 208 aa, Selaginella moellendorffii
MSSFRSLMDPPLFARYMICLKTFLDSLVEEASLRSAKSIPSLEKYQLLRRGTVFVEGAGGIMCEFCMDLKLDKVADHVLFSFRHEMKIKCFHNYLCVIFCHSPNNASFQEAVDKVCKMIQETEAKILQLQKKLMKMGEETGNKDLVDYATWYPCFTSGHLRWVYVTGRYHGLDNPLLNGEPFHGTWFLHPEATFILPFGSKCGFINTM

SEQ ID NO: 57 - protein, 384 aa, Selaginella moellendorffii
MALPSLLSTKLKPLELLSGVTHYDLPPIPCSLPVKCHPQFAKFSRIADTWAIDAMQLQNDPCGKLKAVQSRAPLLYCFLVPFGIGEEEMIAGCKYSWSTSFVDDPFDEETDLKRAKEWKKVVLRAANGTPSAEDLMIRTIKAYSEIMMHLQQMMAAPVFSRFMRAHYAWADHCVELVRRRQHKDPPTVATYLADRCENLLVEPIFILAEVCMKLQIDPEFLSLPEFKKIWTTMLEHAAIVNDVLSIRVDILNGHYYTYPGLVFQQHPEIQTFQEAVDYSVGMIQTKERKFIKLHEMLTDKARQCGFKNKSDLLKYVEALPNFIAGNLYWHYLSARYFGVNNPFLTGEPVQGTILIHPRNTVVLPPYQRNKHPFLIDVDNLELGA

SEQ ID NO: 58 - protein, 358 aa, Selaginella moellendorffii
MRSFSSFHISPMKCKPALRVHPLCDKLQMEMDRWCVDFASPESSDEEMRSFIAQKLPFLSCMLFPTALNSRIPWLIKFVCWFTLFDSLVDDVKSLGANARDASAFVGKYLETIHGAKGAMAPVGGSLLSCFASLWQHFREDMPPRQYSRLVRHVLGLFQQSASQSRLRQEGAVLTASEFVAGKRMFSSGATLVLLMEYGLGVELDEEVLEQPAIRDIATTAIDHLICVNDILPFRVEYLSGDFSNLLSSICMSQGVGLQEAADQTLELMEDCNRRFVELHDLITRSSYFSTAVEGYIDGLGYMMSGNLEWSWLTARYHGVDWVAPNLKMRQGVMYLEEPPRFEPTMPLEAYISSSDSC

SEQ ID NO: 59 - protein, 366 aa, Selaginella moellendorffii
MEAIVSSSKIHAVEHLLSLKSYSLPQILLAHPVKCHPDYTSICKESDEWIFSYLGVTSPEHKKRLAQWRVPIFAAFLTPPSSPKRRTLLGGKFTWLITALDDQLDESKISQAGRSCQYRDAILSIFSGRSDYPAILPAEVPLLRACEELMPEIRSFMLPPTLNRFLAYTKQWSQTFDVAYESTQVFKELRRDNVWITAYFPMIEMFLGLGLGDDVAGSKDFLAAQDAISDHAWMVNDLFSFAKEFRDEKKLSNILSVSLLMDSCVHTIQDAIDLLCTELQAKEEEFLYYHGILVKRAQAGNNQDLLRYLEAILAVIPGNLHFHYITARYHGYNNPCVNGEAWHGKVILQPNTLGPPPKPHPYLYDI

SEQ ID NO: 60 - protein, 348 aa, Selaginella moellendorffii
MAVSSIVSIFAAEKRYSIPPVCKLLASPVLNPLYDAKAESQINAWCAEFLKLQPGSEKAVFVQESRLGLLAAYVYPTIPYEKIVPVGKFFAWYFLADDILDSPEISSSDMRNVTTAYKMVLKGKFDEATLPVKNPELLRQMKMLSEVLEELFLHIVDESGRFVDALTKVLDMFEIESSWLRKQIIPNLDTYLWLREITSGVAPCFALIDGLLQLRLEERGVLDHPLIRKVEEIGTHHIALHNDMISFRKEWAKGYYLNAVPILASNCKCGLNEAIGKVASMVEDVEKDFAQTKHEIVSSGLAMKQGVMDYVNGIEVWMAGNVEWAWTSARYHGIGWIPPPEKSATFQL

SEQ ID NO: 61 - protein, 529 aa, Selaginella moellendorffii
MARTLFNDMLKQAALPDIVTFSTLVEGYCNAGLVDDAERLLEEIIASDCSPDVYTYTSLVDSFCKVKRMVEAHRVLKRMAKRGCQPNVVTYTALIDAFCRAGKPTVAYKLLEEMVGINNDVQPNVQELASVGLGTWKRLARCSRDWSATRTARRICSHTGGLCQGKELSKAMEVLEEMTLSRKGRPNAEAYEAVIQELAREGRHEEANALADELLGNKGHLLSVFKIHLGSIHCEHFRSGEKLFHSTKSRLGLLAAYVYPTIPYEKIVPVAKFIAWFFLADDILDSPEISSSDIRYVATAYKMVFKGRFDEATLPVKNPELLRQMKMLAEVLEELSLHIVDESGRFVDAMTKVLDMFEIESSWLRKQIIPNLDTYLWLREITSGVAPCFALIDGLLQLRLEERGVLDHPLIRKVEEIGTHHIALHNDLILLRKEYFLASDYDVDLPSSEASSTLFFLLQMATFMKYFLEDLCSHFAARCRIIPYKNVSSLWMDQSGAVLQKKLLKLEFTTLFEYLQRLSPTSTSPGTPW

SEQ ID NO: 62 - protein, 348 aa, Selaginella moellendorffii
MAVSSIASIFAAEKSYSIPPVCQLLVSPVLNPLYDAKAESQIDAWCAEFLKLQPGSEKAVFVQESRLGLLAAYVYPTIPYEKIVPVGKFFASFFLADDILDSPEISSSDMRNVATAYKMVLKGRFDEATLPVKNPELLRQMKMLSEVLEELSLHVVDESGRFVDAMTRVLDMFEIESSWLRKQIIPNLDTYLWLREITSGVAPCFALIDGLLQLRLEERGVLDHPLIRKVEEIGTHHIALHNDLMSLRKEWATGNYLNAVPILASNRKCGLNEAIGKVASMLKDLEKDFARTKHEIISSGLAMKQGVMDYVNGIEVWMAGNVEWGWTSARYHGIGWIPPPEKSGTFQL

SEQ ID NO: 63 - protein, 396 aa, Selaginella moellendorffii
MAVSSIASIFAAEKSYSIPPVCQLLVSPVLNPLYDAKAESQIDAWCAEFLKLQPGSEKPVFVQESRLGLLAAYVYPTIDCSDDILDSPEISSSDMTNVATAYKMVLKGRFDEAMLPVKNPDLLRQMKMLSEVLEELSLHVVDESGRFVDAMTRVLDMFEIESSWLRKQIIPNLDTYLWLREITSGVAPCFALIDGLLQLRLEERGVLDHPLIRKVEEIGTHHIALHNDLMSLRKERATGNYLNAVPILASNRKCGLNEAIGKVASMLEDLEKDFARTKHEIISSGLAMKQGVMDYVNGIECFRNSYLSSVFDLNKQIEMHGRCGNIKHAAQIFHASCCDFPSWEASSTLFFLLQMPFCRSLPDNPWAVLLKKLLKLEFTTLFEYLQLTSTSPGTPW

SEQ ID NO: 64 - protein, 368 aa, Selaginella moellendorffii
MEATLISKFSTVTHFELPQLPNNIPFAYHPQSATISAQIDEWMLRKMKITDQSARKKMIHSKMGLYACMMHPNAEREKLVLAGKNLWALLLMDDLLESSSKEEMPRLNTTISSLGSGNSGDGAIRNPVLLLYKEVLGELRAAMEPPLLDRYLHCLAASLEGVRKQVHHRTKKSVPGPEEYKFTRRANGFMDILGGIMTEFCMGIRLNQAQIQSPTFRELLNSVSDYVILVNDLLSFRKEFYGGDYHHNWISVLSYHGPSGISFQDVIDQLCEMIQAEEHSILALQKKIADEEGCDSELTKFASELAMVASGSLVWSYLSGRYHGYDNPLITGEIFSGTWLLHPVATVVLPSIKARDTLLGLKVPVPLP

SEQ ID NO: 65 - protein, 366 aa, Selaginella moellendorffii
MEDVLVSRILGVTHFELPLLPNNIAFYCHPEFQSISLQIDEWFLDKMRIADETSKKKVLESRIGLYACMMHPHAEREKIVLAGKHLWAVFLLDDLLESSGTQEMPKLNATISDLASGNSNEDVTNPVLVLYREVMEEIRAGMEPPLLDRYVECLGASLEAVKDQVHHRAEKSIPGVEAYKLARRATGFMEAVGGIMTEFCMGIRLNESQIQSPVFRELLNSVSDHVVLVNDLLSFRKEFYEGACHHNWISVLLQHSPSGTRFQDVIDQLCEMIQEEELSILALQRKISSKENSDSELMKFAREFPMVASGSLVWSYVTGRYHGYGNPLLTGEIFSGTWLLHPMATVVLPKSTVFSLNHLVYSHVIL

SEQ ID NO: 66 - protein, 380 aa, Selaginella moellendorffii
MEDILVSRISGVTHFELPLLPNNIAFYCHPEFQSISLQIDEWFLAKMRITDETSKKKVLESRIGLYACMMHPHAEREKIVLAGKHLWAVFLLDDLLESSGTQEMPKLNATIFNLASGNSNEDVTNPVLVLYREVMEEIRAGMEPPLLDRYVECLGASLEAVKDQVHHRVEKSIPGVEEYKLARRATGFMEAVGGIMTEFCMGIRLNESQIQSPVFRELLNSVSDHVVLVNDLLSFRKEFYEGACHHNWISVLLQHSPRGTRFQDAIDQLCEMIQEKELSILALQRKISSKEHSDSELMKFAREFPMVASGSLVWSYVTGRYHGYGNPLLTGEIFSGTWLLHPMATVNGYQTILVYSLINNTEIKSIISTIYTVSQIASSG

SEQ ID NO: 67 - protein, 368 aa, Selaginella moellendorffii
MKDLFRISGVTHFELPLLPNNIPFACHPEFQSISLKIDKWFLGKMRIADETSKKKVLESRIGLYACMMHPHAKREKLVLAGKHLWAVFLLDDLLESSSKHEMPQLNLTISNLANGNSDEDYTNPLLALYREVMEEIRAAMEPPLLDRYVQCVGASLEAVKDQVHRRAEKSIPGVEEYKLARRATGFMEAVGGIMTEFCIGIRLSQAQIQSPIFRELLNSVSDHVILVNDLLSFRKEFYGGDYHHNWISVLLHHSPRGTSFQDVVDRLCEMIQAEELSILALRKKIADEEGSDSELTKFAREFPMVASGSLVWSYVTGRYHGYGNPLLTGEIFSGTWLLHPMATVVLPSKFRMDTMRFSLAPKKRDSFP

SEQ ID NO: 68 - protein, 356 aa, Selaginella moellendorffii
MEATLISKFSTVTHFELPQLPNNIPFAYHPQSATISPQIDEWMLRKMKITDQSVRKKMIHSKIGLYACMMYPNAEREKLVLAGKNLWALLLIDDLLESSSKEEMPRLNTTITNLGSGNSRDGAIRNPVLLLYKEVLGELRAAMEPPLLDRYLHCLAASLEGVRKQVHHRTRKSVPGPEEYKLTRRANGFMDILGGIMTEFCMGIRLNQAQIQSPTFRELLNSVSDYVILVNDLLSFRKEFYGGDYHDNWISVLSYHGPRGISFQDVIDQLCEMIQAEEHSILALQKKIADEEGCDSELTKFASELAMVASGSLVWSYLSGRYHGYDNPLITGEIFSGTWLLHPVATVVFPSIKARP

SEQ ID NO: 69 - protein, 178 aa, Selaginella moellendorffii
MFEDVMLSIQSLMDPPLFARYMICLRNYLDALVEDSSLRFAKSIPSLTKHQLLRKQLEALYRDKHYSYLCVIFCHDNASFQGTVDKACEMIQETEGEILQLQKKLMKLGEETGNKDLVEYARYPCVASRNLRWSYVTRTSSREPFHATWFLLPEVTLIVPFGSKCGDHPFAITENHLV

SEQ ID NO: 70 - protein, 369 aa, Selaginella moellendorffii
MEDVLAERLSRVSKFDLPSIPCSIPLESHPEFSRISEVTDAWAIRMLGITDPYERQKAIQARFGLLTALATPRGESSKLEVASKHFWTFFVLDDIAETDFGEEEGQKAADILLEVAEGSYVFSEKEKQKNPSYAMFEEVMSSFRSLMDPPLFARYMTCLKNFLDSVVEEASLRFAKSIPSLEKYQLLRRETVFVEASGGIMCEFCMDLKLDKGVVESPEFVAFVKAVVDHAALVNDLLSFRHEMKIKCFHNYLCVIFFHSPDNASFQETVDKVCKMIQETEAEILQLQKKVMKMGVETGNKDLVEYATWYPCFASGHLRWSYVTGRYHGLDNPLLNGEPFHGTWFLHPEVTLMLPFGAKCGDHPWIARS

SEQ ID NO: 71 - protein, 367 aa, Selaginella moellendorffii
MEDVLAEKLSRVCKFDLPFIPCSIPFECHPDFTRISKDTDAWALRMLSITDPYERKKALQGRHSLYSPMIIPRGESSKAELSSKHTWTMFVLDDIAENFSEQEGKKAIDILLEVAEGSYVLSEKEKEKHPSHAMFEEVMSSFRSLMDPPLFARYMNCLRNYLDSVVEEASLRIAKSIPSLEKYRLLRRETSFMEADGGIMCEFCMDLKLHKSVVESPDFVAFVKAVIDHVVLVNDLLSFRHELKIKCFHNYLCVIFCHSPDNTSFQETVDKVCEMIQEAEAEILQLQQKLIKLGEETGDKDLVEYATWYPCVASGNLRWSYVTGRYHGLDNPLLNGEPFQGTWFLHPEATLILPLGSKCGNHPFIMI

SEQ ID NO: 72 - protein, 367 aa, Selaginella moellendorffii
MEDVLAEKLSRVCKFDLPFIPCSIPFECHPDFTRISKDTDAWALRMLSITDPYERKKALQGRHSLYSPMIIPRGESSKAELSSKHTWTMFVLDDIAENFSEQEGKKAIDILLEVAEGSYVLSEKEKEKHPSHAMFEEVMSSFRSLMDPPLFARYMNCLRNYLDSVVEEASLRIAKSIPSLEKYRLLRRETSFMEADGGIMCEFCMDLKLHKSVVESPDFVAFVKAVIDHVVLVNDLLSFRHELKIKCFHNYLCVIFCHSPDNTSFQETVDKVCEMIQEAEAEILQLQQKLIKLGEETGDKDLVEYATWYPCVASGNLRWSYVTGRYHGLDNPLLNGEPFQGTWFLHPEATLILPLGSKCGNHPFITI

SEQ ID NO: 73 - protein, 353 aa, Selaginella moellendorffii
MEFLLGKIVPRFELPLLPNNIPCACHPDSSSLSQELDEWFISKLGITDESAQKKIVQSRIMIFACLMHPNGERDRVLLAGKHLWVCFLVDDILESSTREAYGSLKSIVWSIATTGIYKASNEEHDHCLVLLLYQEVLAELRKKMPSSLFTRYCKILSSYLDGVEEEVKHQVKNTIPSSEEYRLLRRRTGFMEVMACIMTEFCVGIKLEESVVNLGEIRKLVKVMDDHIVMVNDLLSLRKEYYSSTICHNWVFVLLADGCGTFQESVDHVCEMIKQEEGSILDLQQKLIIKAKVDKNPELLKFACNVPMAVAGHLKWSFITARYHGCDNALLNGELFHGTWLMDPNQTIIQKNI

SEQ ID NO: 74 - protein, 348 aa, Selaginella moellendorffii
MAVSSIASIFAAEKSYSIPPVCQLLVSPVLNPLYDAKAESQIDAWCAEFLKLQPGSEKAVFVQESRLGLLAAYVYPTIPYEKIVPVGKFFASFFLADDILDSPEISSSDMRNVAIAYKMVLKGRYDEATLPVKNPELLRQMKMLSEVLEELSLHVVDESGRFVDAMTRVLDMFEIESSWLRKQIIPNLDTYLWLREITSGVAPCFALIDGLLQLRLEERGVLDHPLIRKVEEIGTHHIALHNDLMSLRKEWASGNYLNAVPILASNRKCGLNEAIGKVASMVEDLEKDFAQTKHEIISSGLAMKQGVMDYVNGIEVWMAGNVEWGWTTARYHGIGWIPPPEKSGTFQL

SEQ ID NO: 75 - protein, 353 aa, Selaginella moellendorffii
MECLMAKLVPRLELPLLPNNIPSACHWDSSSLSQELDQWLISKLGITDESAKRKIVQSRVMLLACLMHPNGERDRVLLAGKHLWVYFLVDDILESSSREGYGALKSIVWSIATTGIYKASEEHDHHDLVLLLLVEVMVELRKEMPTSLFARYCKILSIYLDSVQEEVKHQINNTIPSSEEYRLLRRRTGFMEVMACIMTEFCVGINLEELVVNLGEIRELVKIMDDHIVTVNDLLSLRKEYYNGTIYHNWVIVLLAHDCATFQKSVDRVCEMIKQEEDSILDLQKKLIIKAKVDKNPELLKFAFNVPMAVAGHLKWAFITARYHGCDNALLDGELFHGTWIMDPNQTVIVKNM

SEQ ID NO: 76 - protein, 209 aa, Selaginella moellendorffii
MGMLNDVYTDLKGFMKPGHKTRFSNSMIDVLDMFEVESSWLHKKLVPNFEIYMWMREVTAGVIPCMVAIDFLNNFGLEEEGMLDDLHIQTLEVIANRHSFLANDMVSFKKEWACEQYLNSVALVGYSSNCGLNEAMEKVAEMVQDLEKEFADIKQKVLSNKDLNKGNVMGYVQGLEYFMAGNIEFSWLSARYHGVGWVSPAEKYGTLEF

SEQ ID NO: 77 - protein, 372 aa, Selaginella moellendorffii
MASPCLQKLPAVEHLFALTRFELPEIPCSLSFQRHPEYMSITKEANEWAFKCMRRDFSPEEKKCLVQWKVPMFTCLSTPHAPKANMVASAKFAWLTAFLDDPFDDNEVAGGALATSYLDTVLSLCYGTASLAEIPDILAYRACHDLMKDLRSLLKPKLFKRTVSTVEGWARSISSDDLTQDYELYRRKNVFILPLIYAMGASFDDEDVESLDYIRAQNAMLDHMWMVNDVFSFPKEFYKKKFNNLPAVLLLTDPSVQTFQDAVNTTCRMIQDKEDEFIYYRDILATNASRNGKKDFLKFLDVLSCAIPANLVFHYASSRYHGMDNPLLGGPTFSGTWILDPKRTIILSDPKRWNVVASSNKLNQIQNLSNLI

SEQ ID NO: 78 - protein, 177 aa, Selaginella moellendorffii
MGALFDDEDVESLDYISAQNAMLDHMWMVNDVFSFLKEFYKNKFNNLPAVLLTDQSVQTFQDAVNTTWRMIQDKEDEFIFYRDILAANASRNGKKDFLKFLDVLSCAIPANLVYASSHYHGVDNLLSGGTFRGTWILDPKRTIIVSDPKSCNVVATTDEVKINVSYAWLFVILILAN

SEQ ID NO: 79 - protein, 367 aa, Selaginella moellendorffii
MGSLCLQKLSAVERLFALESFELPEVPCSLSFHRHPEYKSITREANEWAFKCTRRDLSPEEKKSLLQWKVPMVTCLSTAHAPKENMVASAKFAWAIAFLDDPIDDNEVAATSYLDTVLSLCNGTASLAEVPDIVAYRACHDLMKDLRSLLQPELFKRTVSTVEGWARSISSDDLKQDYKLYRRNNIFILPLFYTLIGASFEDEDVESPDFVSAQNAMLDHIWMVNDIFSFRNEFYKKKLNNLPAVLLLTDPSVQTFQEAVNATCRMIQDKEEEFIYYRNILAANASRNGKDFLKFLDVLSCAIPANLAFHYASSRYHGMDNPLLAGGTFHGTWILDPKRTIIVSDPNRSNGAASNKLNHIQDLSKLI

SEQ ID NO: 80 - protein, 372 aa, Selaginella moellendorffii
MAVYKQGSGFKTEASVILGVTHFELPLLPNNIAFYCHPEFQSISLQIDEWFLDKMRIADETSKKKVLESRIGLYACMMHPHAEREKIVLAGKHLWAVFLLDDLLESSGTQEMPKLNATISDLASGNSNEDVTNPVLVLYREVMEEIRAGMEPPLLDRYVECLGASLEAVKDQVHHRAEKSIPGVEAYKLARRATGFMEAVGGIMTEFCMGIRLNESQIQSPVFRELLNSVSDHVVLVNDLLSFRKEFYEGACHHNWISVLLQHSPSGTRFQDVIDQLCEMIQEEELSILALQRKISSKENSDSELMKFAREFPMVASGSLVWSYVTGRYHGYGNPLLTGEIFSGTWLLHPMATVVLPKSTVFSLNHLVYSHV

SEQ ID NO: 81 - protein, 371 aa, Selaginella moellendorffii
MASPCLQKLPAVEHLFALTPEIPFQRHPEYMSITKEANEWAFKCMRRDFSPEEKKCLVQWKVPMFTCLSTPHAPKANMVASAKFAWLTAFLNDPFDDNEVAAGALATSYLDTVLSLCYGTASLAEVPDILAYRACHDLMEDLRSLLKPELFKRTVSTVEGWARSISSDDLTQDYELYRRKNVFILPLIYAMGASFDDEDVESLDYIRAQNAMLDHMWMVNDVFSFPKEFYKKKFNNLPAVLLLTDPSVQTFQDAVNTTCRMIQDKEDEFIYYRDILATNASRNGKKDFLKFLDVLSCTIPANLVFHYASSCYHGMDNPLLGGGTFRGTWILDPKRTIIVSDPKSQAIPHAVHIWKSAVFYAQSYFIQSLED

SEQ ID NO: 82 - protein, 372 aa, Selaginella moellendorffii
MASLCLQKLPAVEHLFALTRFELPEIPCSLSFQRHPEYTSITKEANEWAFKCMRRDFSPEEKKCLVQWKVPMFTCLSTPHAPKANMVASAKFAWLTAFLDDPFDDNEVAGGALATSYLNTVLSLCYGTASLAEVPDILAYRACHDLMKDLRSLLKPELFKRTVSTVEGWARSILSDDLTQDYELYRRKNVFILPLIYAMGASFDDEDVESLDYIRAQNAMLDHMWMVNDVFSFPKEFYKKKFNNLPAVLLLTDPSVQTFQDAVNTTCRMIQDKEDEFIYYRDILATNASWNGKKDFLKFLDVLSCAIPANLVFHYASSRYHGMDNPLLGGGTFRGTWILDPKCTIIVSDPKRCNVVASSNKLNQIQNLSNLI

SEQ ID NO: 83 - protein, 321 aa, Selaginella moellendorffii
MPGEYSFYNFLDMGFAPYGDYWKNMRKLCATGTIPSRREKIGPYLLDSARRERWGFLPKRCDLTTTGSNIFPTQSNLCYGTASLAEVPDILAYRACHDLMKDLRSLLKTELFRRTVSTVEGWARSILSDDLTQDYELYRRKNVFILPLIYAMGASFDDEDVESLDYIRAQNAMLDHMWMVNDVFSFPKEFYKKKFNNLPAVLLLTDPSVQTFQDAVNTTCRMIQDKEDEFIYYCDILASVPEWEESFPEVPGCSLLRNPANLVFHYASSRYHMDNPLGGGTFCGTWILDPKRTIIMSDPRRCNVVASSNKLNQIQNLSNLI

SEQ ID NO: 84 - protein, 346 aa, Selaginella moellendorffii
MAFVVEKIPAMEHHLGLKRFYLPPIRCSIPSSAWDPDHKLVAKLANEWAFPFINPSMSDAQKLSLERMRIPLYMSMLVPCGSTESAVLCGKFAWFGTMLDDLLEDESPGGAPREEFLETFQGILHGTHPHRDPVHPSLEFCADLIPRLRSSMAPRVYAHWVAQMEAYAASMDRSVLSLAQSASTVESYLARRRLDCFLLPCFPFIEMSLEIALPDSDLESRDYLALQNAINDHVLLVNDVISFPAELRAKKPLRSIASLQLLLDPSVNTFQDSVDRTCAMIQEKEREVTHYYDVVMRNAVASGNAELVSYLQILKMCVPNNLKFHFISSRYGVNDAESGHGIWIVL

SEQ ID NO: 85 - protein, 441 aa, Selaginella moellendorffii
MGYVGVNMEVLVDCRNTVFAKGLTSLEELWWWCFGRHGFLTQCTLKRRLILSKGTCRQLSITNRPFSLYISWRVLPRHYIAYTALEKHRRRSIMGASSILSIFEGAKSFYIPPHSSYHVDLNPAYDAKLDAEIDKWCMDFLNLHDLTDHKTQFAIQSKLGKLAGFAYQAISSERLSPIAKFFCWLFLADDFMDDPSVPVSDLKNATLAYKLIFKNDYDQAITLVESKGLLRQMGMLNDVYTDLKGFMNPGHKTRFSKSMIDVLDMFEVESSWLHKTLVPNFEIYMWMREVTAGVIPCMVAMDFLNNFGLEEEGVLDDPHIQTLEVIANRHSFLANDMVSFKKEWACEQYLNSVALVGYSSNCGLNEAMEKVAQMVQDLEKEFADIKQKVLSNKDLNKGNVMGYVQSLEYFMAANIEFSWISARYHGVGWVSPAEKYGTFEF

SEQ ID NO: 86 - protein, 348 aa, Selaginella moellendorffii
MGPSSILSIFEGAKSFYIPPHSSYHVDLNPAKLDAEIDKWCMDFLNLHDLTDHKTQFAIQSKLGKLAGLAYQAISSERLRPMAKFLCWLFLADDFMDNPSVPVSDLKNATLAYKLIFKNDYDQAITLVESKDLLRQMGMLNDVYTDLKGFMNPGHRTRFSKSMIDVLDMFEVESSWLHKKLVPNFEIYNVTAGVIPCMVAIDFLNNFGLEDDVLDHPNIQRLEVIANRHTYLANDMVSFKKEWACDMYLNSVALVGYSSNCGLHEAMEKVAQMVQDLEKEFADIKQKVLSNKDLNKGNVMGYVQGLEYFMAGNIGSLRDIMGWDGFHQLRNMVPWSSSLLLLALEAGA

SEQ ID NO: 87 - protein, 323 aa, Selaginella moellendorffii
MAAPSIYRPQILEQLLACKSIYLPQIRCSLPLQCHPDYASVSRQANDWAFRFLKINATNAAAEKKCFTQWRTPLYGTFVVPWGDSRHALAAAKYTWLITILDDAVDEEPSQRNEILEAYMSLASGNLLAATRTKRSSRKSLNAVHRREDFVVKPMLNFTQMCLGVKLRDKDLESEEYLRAIDAMFDHIWLVNDIFSFPKELRKKTFKNIIFLLLFTDHTVRSVQQAVDKANAMVQEKEQEFMYYHEILTRKAMESGNHDFLAYLRAIPAFIPGNLRWHYLTARYHGVDNPFVTGEPFSGTWLFHDTQTIILPEYKPTHPHLQV

SEQ ID NO: 88 - protein, 366 aa, Selaginella moellendorffii
MAAPSIYRPQILEQLLACKSIYLPQIRCSLPLQCHPDYASVSRQANDWAFRFLKINATNAAADKKYFTQWRMPLYGTFVVPWGNSRHALAAAKYTWLITILDDAVDEEPSQRDEILEAYMSLASGQRSIAQVPNKPVLVAQAELVPDLRKLMSPLLFQRLLVSYRKFVGCYSAKVDEEEFTKESYAVHRREDYVVKPMLNFTQMCLGVELRDKDLESEEYLRAIDAMFDHMWLVNDIFSFPKELRKKTFKNIIFLLLFTDHTVRSVQQAVDKANAMIQEKEQEFMYYHEILTRKAMESGNHDFLAYLRAIPAFIPGNLRWHYLAARYHGVDNPFVTGEPFSGTWLFHDTQTIILPEYKPTHPHLQV

SEQ ID NO: 89 - protein, 156 aa, Selaginella moellendorffii
MGMLNDVYTDLKGFMNPGHKTQFSNSMIDVLDMFEVESSWLHKKLVPNFEIYMWMREEWACEQYLNSVALVGYSSNCGLNKAMEKVAEMVQDLEKEFADIKQKVLSNKDLNKGNVMGYVQSLEYFMAANIEFSWISARYHGVGWVSPAEKYGTLEF

SEQ ID NO: 90 - DNA, 21 bp, Selaginella moellendorffii
ggctattctatccattgtgag

SEQ ID NO: 91 - DNA, 20 bp, Selaginella moellendorffii
tcacaccacacattgatctc

SEQ ID NO: 92 - DNA, 20 bp, Selaginella moellendorffii
gaaagttcttcgcttcgttc

SEQ ID NO: 93 - DNA, 20 bp, Selaginella moellendorffii
tgtatgcagttgccacattc

SEQ ID NO: 94 - DNA, 20 bp, Selaginella moellendorffii
tttccaggacgtagttgacc

SEQ ID NO: 95 - DNA, 20 bp, Selaginella moellendorffii
caaacttagtcagctctgag

SEQ ID NO: 96 - DNA, 20 bp, Selaginella moellendorffii
cgttcttgtcaatgatctcc

SEQ ID NO: 97 - DNA, 20 bp, Selaginella moellendorffii
cataccttgtccacagtctc